SEQUENCE LISTING (24 sequences)

SEQ ID NO: 1 - protein, 140 aa, Artificial Sequence
  Synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 176 aa, Artificial Sequence
  Synthetic construct
DLNKVFPPEVAVFEPSEAEISHTQKATLVCLATGFFPDHVELSWWVNGKEVHSGVSTDPQPLKEQPALNDSRYCLSSRLRNHFRCRVSATFWQNPQVQFYGLSENDEWTQDRAKPVTQIVSAEAWGRADCGFTSVSYQQGVLSATILYEILLGKATLYAVLVSALVLMAMVKRKDF

SEQ ID NO: 3 - protein, 178 aa, Artificial Sequence
  Synthetic construct
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 4 - protein, 18 aa, Artificial Sequence
  Synthetic construct
MALPVTALLLPLALLLHA

SEQ ID NO: 5 - protein, 21 aa, Artificial Sequence
  Synthetic construct
MDFQVQIFSFLLISASVIMSR

SEQ ID NO: 6 - protein, 8 aa, Artificial Sequence
  Synthetic construct
DYKDDDDK

SEQ ID NO: 7 - protein, 116 aa, Artificial Sequence
  Synthetic construct
MAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVNARLMFGDGTQLVVKPN

SEQ ID NO: 8 - protein, 138 aa, Artificial Sequence
  Synthetic construct
IQNDPAVYQLRDKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 9 - protein, 119 aa, Artificial Sequence
  Synthetic construct
MEADIYQTPRYLVIGTGKKITLECSQTMGHDKMYWYQQDPGMELHLIHYSYGVNSTEKGDLSSESTVSRIRTEHFPLTLESARPSHTSQYLCASSEARGLAEFTDTQYFGPGTRLTVLE

SEQ ID NO: 10 - protein, 176 aa, Artificial Sequence
  Synthetic construct
LKNVFPPEVVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - protein, 118 aa, Artificial Sequence
  Synthetic construct
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSS

SEQ ID NO: 12 - protein, 107 aa, Artificial Sequence
  Synthetic construct
DIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIK

SEQ ID NO: 13 - protein, 261 aa, Artificial Sequence
  Synthetic construct
MDFQVQIFSFLLISASVIMSRQIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSGGGGSGGGGSGGGGSDIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIK

SEQ ID NO: 14 - protein, 261 aa, Artificial Sequence
  Synthetic construct
MDFQVQIFSFLLISASVIMSRDIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIKGGGGSGGGGSGGGGSQIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSS

SEQ ID NO: 15 - protein, 231 aa, Artificial Sequence
  Synthetic construct
MAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVNARLMFGDGTQLVVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLS

SEQ ID NO: 16 - protein, 263 aa, Artificial Sequence
  Synthetic construct
MEADIYQTPRYLVIGTGKKITLECSQTMGHDKMYWYQQDPGMELHLIHYSYGVNSTEKGDLSSESTVSRIRTEHFPLTLESARPSHTSQYLCASSEARGLAEFTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSA

SEQ ID NO: 17 - protein, 153 aa, Artificial Sequence
  Synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 18 - protein, 219 aa, Artificial Sequence
  Synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 19 - protein, 263 aa, Artificial Sequence
  Synthetic construct
SNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 308 aa, Artificial Sequence
  Synthetic construct
TTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 135 aa, Artificial Sequence
  Synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVNARLMFGDGTQLVVKPN

SEQ ID NO: 22 - protein, 137 aa, Artificial Sequence
  Synthetic construct
MTIRLLCYVGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEARGLAEFTDTQYFGPGTRLTVLE

SEQ ID NO: 23 - protein, 250 aa, Artificial Sequence
  Synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVNARLMFGDGTQLVVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLS

SEQ ID NO: 24 - protein, 281 aa, Artificial Sequence
  Synthetic construct
MTIRLLCYVGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEARGLAEFTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSA

(19) United States
(12) Patent Application Publication
(10) Pub. No.: US 2023/0381235 A1
(43) Pub. Date: Nov. 30, 2023

(54) MULTIPLEXED ENGINEERED IPSCS AND IMMUNE EFFECTOR CELLS TARGETING SOLID TUMORS

(71) Applicant: Fate Therapeutics, Inc., San Diego, CA (US)

(72) Inventors: Bahram VALAMEHR, San Diego, CA (US); Tom T. LEE, San Diego, CA (US); Bi-Huei YANG, San Diego, CA (US); Mochtar PRIBADI, San Diego, CA (US); Chia-Wei CHANG, San Diego, CA (US)

(21) Appl. No.: 18/250,737

(22) PCT Filed: Nov. 4, 2021
(86) PCT No.: PCT/US2021/072255

§ 371 Date: Apr. 26, 2023

Related U.S. Application Data
(60) Provisional application No. 63/109,842, filed on Nov. 4, 2020.
(60) Provisional application No. 63/228,490, filed on Aug. 2, 2021.

(51) Int. Cl. A61K 35/17; A61K 39/00; A61P 35/00; C07K 14/725; C07K 14/705; C07K 14/74; C12N 5/0783

(52) U.S. Cl. CPC A61K 35/17; A61K 39/4631; A61P 35/00; C07K 14/7051; C07K 14/70521; C07K 14/70539; C12N 5/0636; C12N 5/0646

(57) ABSTRACT

Provided are methods and compositions for obtaining functionally enhanced derivative effector cells obtained from directed differentiation of genomically engineered iPSCs. The iPSC-derived cells provided herein have stable and functional genome editing that delivers improved or enhanced therapeutic effects. Also provided are therapeutic compositions and the use thereof comprising the functionally enhanced derivative effector cells alone, or with antibodies or checkpoint inhibitors in combination therapies.

### RELATED APPLICATIONS

This application claims priority to U.S. Provisional Application Ser. No. 63/109,842, filed Nov. 4, 2020, and to U.S. Provisional Application Ser. No. 63/228,490, filed Aug. 2, 2021, the disclosure of each of which is hereby incorporated by reference in their entireties.

### FIELD OF THE INVENTION

The present disclosure is broadly concerned with the field of off-the-shelf immunocellular products. More particularly, the present disclosure is concerned with strategies for developing multifunctional effector cells capable of delivering therapeutically relevant properties in vivo. The cell products developed under the present disclosure address critical limitations of patient-sourced cell therapies.

### REFERENCE TO SEQUENCE LISTING SUBMITTED ELECTRONICALLY

This application incorporates by reference a Computer Readable Form (CRF) of a Sequence Listing in ASCII text format submitted with this application, entitled 184143-633601_SequenceListing_ST25.txt, which was created on Nov. 4, 2021, and is 37,739 bytes in size.

### BACKGROUND OF THE INVENTION

The field of adoptive cell therapy is currently focused on using patient- and donor-sourced cells, which makes it particularly difficult to achieve consistent manufacturing of cancer immunotherapies and to deliver therapies to all patients who may benefit. There is also a need to improve the efficacy and persistence of adoptively transferred lymphocytes to promote favorable patient outcomes. Lymphocytes such as T cells and natural killer (NK) cells are potent anti-tumor effectors that play an important role in innate and adaptive immunity. However, the use of these immune cells for adoptive cell therapies remains challenging and has unmet needs for improvement. Therefore, there are significant opportunities to harness the full potential of T and NK cells, or other immune effector cells in adoptive immunotherapy.

### SUMMARY OF THE INVENTION

There is a need for functionally improved effector cells that address issues ranging from response rate, cell exhaustion, loss of transfused cells (survival and/or persistence), tumor escape through target loss or lineage switch, tumor targeting precision, off-target toxicity, off-tumor effect, to efficacy against solid tumors, i.e., tumor microenvironment and related immune suppression, recruiting, trafficking and infiltration.

It is an object of the present invention to provide methods and compositions to generate derivative non-pluripotent cells differentiated from a single cell derived iPSC (induced pluripotent stem cell) clonal line, which iPSC comprises one or several genetic modifications in its genome. Said one or several genetic modifications include DNA insertion, deletion, and substitution, and which modifications are retained and remain functional in subsequently derived cells after differentiation, expansion, passaging and/or transplantation.

The iPSC derived non-pluripotent cells of the present application include, but not limited to, CD34⁺ cells, hemogenic endothelium cells, HSCs (hematopoietic stem and progenitor cells), hematopoietic multipotent progenitor cells, T cell progenitors, NK cell progenitors, T cells, NKT cells, NK cells, B cells, and immune effector cells having one or more functional features that are not present in a primary NK, T, and/or NKT cell. The iPSC-derived non-pluripotent cells of the present application comprise one or several genetic modifications in their genome through differentiation from an iPSC comprising the same genetic modifications. The engineered clonal iPSC differentiation strategy for obtaining genetically engineered derivative cells requires that the developmental potential of the iPSC in differentiation is not adversely impacted by the engineered modality in the iPSC, and also that the engineered modality functions as intended in the derivative cell. Further, this strategy overcomes the present barrier in engineering primary lymphocytes, such as T cells or NK cells obtained from peripheral blood, as such cells are difficult to engineer, with engineering of such cells often lacking reproducibility and uniformity, resulting in cells exhibiting poor cell persistence with high cell death and low cell expansion. Moreover, this strategy avoids production of a heterogenous effector cell population otherwise obtained using primary cell sources which are heterogenous to start with.

Some aspects of the present invention provide genome-engineered iPSCs obtained using a method comprising (I), (II) or (III), reflecting a strategy of genomic engineering subsequently to, simultaneously with, and prior to the reprogramming process, respectively:
(I): genetically engineering iPSCs by one or both of (i) and (ii), in any order: (i) introducing into iPSCs one or more construct(s) to allow targeted integration at selected site(s); (ii) (a) introducing into iPSCs one or more double stranded break(s) at selected site(s) using one or more endonucleases capable of selected site recognition; and (b) culturing the iPSCs of step (I)(ii)(a) to allow endogenous DNA repair to generate targeted in/dels at the selected site(s), simultaneously or sequentially; thereby obtaining genome-engineered iPSCs capable of differentiation into partially or fully differentiated cells.
(II): genetically engineering reprogramming non-pluripotent cells to obtain the genome-engineered iPSCs comprising: (i) contacting non-pluripotent cells with one or more reprogramming factors, and optionally a small molecule composition comprising a TGFβ receptor/ALK inhibitor, a MEK inhibitor, a GSK3 inhibitor and/or a ROCK inhibitor to initiate reprogramming of the non-pluripotent cells; and (ii) introducing into the reprogramming non-pluripotent cells of step (II)(i) one or both of (a) and (b), in any order: (a) one or more construct(s) to allow targeted integration at selected site(s); (b) one or more double stranded break(s) at a selected site using at least one endonuclease capable of selected site recognition, then the cells of step (II)(ii)(b) are cultured to allow endogenous DNA repair to generate targeted in/dels at the selected site(s); as such the obtained genome-engineered iPSCs comprise at least one functional targeted genomic editing, and said genome-engineered iPSCs are capable of differentiation into partially or fully differentiated cells.
(III): genetically engineering non-pluripotent cells for reprogramming to obtain genome-engineered iPSCs comprising (i) and (ii): (i) introducing into non-pluripotent cells one or both of (a) and (b), in any order: (a) one or more construct(s) to allow targeted integration at selected site(s); (b) one or more double stranded break(s) at a selected site using at least one endonuclease capable of selected site recognition, wherein the cells of step (III)(i)(b) are cultured to allow endogenous DNA repair to generate targeted in/dels at the selected sites; and (ii) contacting the cells of step (III)(i) with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted editing at selected sites; thereby obtaining genome-engineered iPSCs comprising at least one functional targeted genomic editing, and said genome-engineered iPSCs are capable of being differentiated into partially differentiated cells or fully-differentiated cells.

In one embodiment of the above method, the at least one targeted genomic editing at one or more selected sites comprises insertion of one or more exogenous polynucleotides encoding safety switch proteins, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells therefrom. In some embodiments, the exogenous polynucleotides for insertion are operatively linked to (1) one or more exogenous promoters comprising CMV, EF1α, PGK, CAG, UBC, or other constitutive, inducible, temporal-, tissue-, or cell type-specific promoters; or (2) one or more endogenous promoters comprised in the selected sites comprising AAVS1, CCR5, ROSA26, collagen, HTRP, H11, beta-2 microglobulin, CD38, GAPDH, TCR or RUNX1, or other locus meeting the criteria of a genome safe harbor. In some embodiments, the genome-engineered iPSCs generated using the above method comprise one or more different exogenous polynucleotides encoding protein comprising caspase, thymidine kinase, cytosine deaminase, modified EGFR, or B-cell CD20, wherein when the genome-engineered iPSCs comprise two or more suicide genes, the suicide genes are integrated in different safe harbor locus comprising AAVS1, CCR5, ROSA26, collagen, HTRP, H11, beta-2 microglobulin, CD38, GAPDH, TCR or RUNX1. In one embodiment, the exogenous polynucleotide encodes a partial or full length peptide of IL2, IL4, IL6, IL7, IL9, IL10, IL11, IL12, IL15, IL18, IL21, and/or respective receptors thereof. In some embodiments, the partial or full peptide of IL2, IL4, IL6, IL7, IL9, IL10, IL11, IL12, IL15, IL18, IL21, and/or respective receptors thereof encoded by the exogenous polynucleotide is in the form of a fusion protein.

In some other embodiments, the genome-engineered iPSCs generated using the method provided herein comprise in/dels at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the iPSCs or derivative cells therefrom. In some embodiments, the endogenous gene for disruption comprises at least one of CD38, B2M, TAP1, TAP2, Tapasin, NLRC5, PD1, LAGS, TIM3, RFXANK, CIITA, RFX5, RFXAP, RAG1, and any gene in the chromosome 6p21 region.

In yet some other embodiments, the genome-engineered iPSCs generated using the method provided herein comprise a caspase encoding exogenous polynucleotide at AAVS1 locus, and a thymidine kinase encoding exogenous polynucleotide at H11 locus.

In still some other embodiments, approach (I), (II) and/or (III) further comprises: contacting the genome-engineered iPSCs with a small molecule composition comprising a MEK inhibitor, a GSK3 inhibitor and a ROCK inhibitor, to maintain the pluripotency of the genomic-engineered iPSCs. In one embodiment, the obtained genome engineered iPSCs comprising at least one targeted genomic editing are functional, are differentiation potent, and are capable of differentiating into non-pluripotent cells comprising the same functional genomic editing.

Accordingly, in one aspect, the present invention provides a cell or a population thereof, wherein the cell is an eukaryotic cell, an animal cell, a human cell, an immune cell, an induced pluripotent cell (iPSC), a clonal iPSC or a derivative cell differentiated therefrom, and wherein the cell comprises: (i) a polynucleotide encoding a transgenic TCRα chain (tgTCRα); and (ii) a polynucleotide encoding a transgenic TCRβ chain (tgTCRβ), wherein the tgTCRα chain and the tgTCRβ chain form an exogenous TCR complex (TCR^{exo}) that recognizes a first tumor antigen; and optionally (iii) one or more additional exogenous polynucleotides comprising a polynucleotide encoding a chimeric antigen receptor (CAR) or an engager targeting at least a second tumor antigen. In some embodiments, (i) the polynucleotide encoding the tgTCRα chain and the polynucleotide encoding the tgTCRβ chain are comprised in a bi-cistronic construct, and optionally wherein: (a) the construct is inserted at a constant region of TCRα or TCRβ (TRAC or TRBC); (b) the insertion of the construct disrupts expression of endogenous TCRα or TCRβ at the insertion site; and/or (c) expression of the construct is driven by an endogenous promoter of TCR or an exogenous promoter; or (ii) the polynucleotide encoding the CAR or the engager is inserted at TRAC or TRBC, and optionally wherein: (a) the insertion of the polynucleotide encoding the CAR or the engager disrupts expression of the endogenous TCRα or TCRβ at the insertion site; and/or (b) expression of the CAR or the engager is driven by an endogenous promoter of TCR or an exogenous promoter; or (iii) the polynucleotides encoding the tgTCRα chain and the tgTCRβ chain, the CAR or the engager, or the one or more additional polynucleotides are inserted in one, or more safe harbor loci or selected gene loci. In particular embodiments, the construct and the polynucleotide encoding the CAR or the engager are each inserted at a constant region of TCRα or TCRβ (TRAC or TRBC), but not at the same constant region, thereby disrupting expression of both endogenous TCRα and TCRβ, knocking out the endogenous TCR, and avoiding a mispaired TCR comprising: (a) the transgenic TCRα and the endogenous TCRβ, or (b) the transgenic TCRβ and the endogenous TCRα. In some embodiments, the construct and the polynucleotide encoding the CAR or the engager are each integrated at a locus comprising a safe harbor locus or a selected gene locus. In some embodiments, (i) the safe harbor locus comprises at least one of AAVS1, CCR5, ROSA26, collagen, HTRP, H11, GAPDH, or RUNX1; (ii) the selected gene locus is one of B2M, TAP1, TAP2, Tapasin, NLRC5, CIITA, RFXANK, RFX5, RFXAP, TCR, NKG2A, NKG2D, CD38, CD25, CD69, CD71, CD44, CD58, CD54, CD56, CIS, CBL-B, SOCS2, PD1, CTLA4, LAG3, TIM3, or TIGIT; and/or (iii) the integration of the exogenous polynucleotides knocks out expression of the gene in the locus.

In some embodiments, the iPSC is a clonal iPSC, a single cell dissociated iPSC, an iPSC cell line cell, or an iPSC master cell bank (MCB) cell; or the derivative cell may comprise a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, a derivative B lineage cell, or a derivative effector cell having one or more functional features that are not present in a counterpart primary T, NK, NKT, and/or B cell. In some embodiments, the derivative effector cell is a hematopoietic cell and comprises longer telomeres in comparison to its counterpart primary cell. In some embodiments, the cell comprises one of the genotypes listed in Table 1; or the cell comprises: (i) (1) a CD19-CAR at TRAC locus, (2) a TRAC knockout, and (3) a MR1-TCR or a NYESO1-TCR, and optionally (4) with or without TRBC knockout; (ii) (1) a BCMA-CAR and hnCD16 insertion at TRAC locus, (2) a TRAC knockout, and (3) a MR1-TCR or NYESO1-TCR, and optionally (4) with or without TRBC knockout; or (iii) (1) a MICA/B B-CAR insertion at TRAC locus, (2) a TRAC knockout, (3) a hnCD16 insertion at CD38 locus, (4) a CD38 knockout, and (5) a MR1-TCR or NYESO1-TCR, and optionally (6) with or without TRBC knockout. In some embodiments, the cell has therapeutic properties comprising one or more of: (i) increased cytotoxicity; (ii) improved persistency and/or survival; (iii) enhanced ability in migrating, and/or activating or recruiting bystander immune cells, to tumor sites; (iv) improved tumor penetration; (v) enhanced ability to reduce tumor immunosuppression; (vi) improved ability in rescuing tumor antigen escape; (vii) controlled apoptosis; (viii) enhanced or acquired ADCC; and (ix) ability to avoid fratricide, in comparison to its counterpart primary cell obtained from peripheral blood, umbilical cord blood, or any other donor tissues without the same genetic edit(s).

In some embodiments, the first tumor antigen and the second tumor antigen each comprises at least one of: (i) MR1, NYESO1, MICA/B, EpCAM, EGFR, B7H3, Muc1, Muc16, CD19, BCMA, CD20, CD22, CD38, CD123, HER2, CD52, GD2, MSLN, VEGF-R2, PSMA and PDL1; or (ii) ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), K-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NYESO1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; and wherein the first tumor antigen and the second tumor antigen are the same or different.

In some embodiments, the first tumor antigen comprises at least one of MR1, NYESO1, and MICA/B; or (i) the tgTCRα comprises a variable alpha (Vα) fragment that has at least about 85% identity to SEQ ID NO: 7, and a TCRα constant fragment comprising a sequence having at least about 85% identity to SEQ ID NO: 8; and/or (ii) the tgTCRβ comprises a variable alpha (Vβ) fragment that has at least about 85% identity to SEQ ID NO: 9, and a TCRβ constant fragment comprising a sequence having at least about 85% identity to SEQ ID NO: 10.

In some embodiments, the CAR is: (i) T cell specific or NK cell specific; (ii) a bi-specific antigen binding CAR; (iii) a switchable CAR; (iv) a dimerized CAR; (v) a split CAR; (vi) a multi-chain CAR; (vii) an inducible CAR; (viii) an inactivation CAR; (ix) co-expressed with a partial or full peptide of a cell surface expressed exogenous cytokine and/or a receptor thereof, optionally in separate constructs or in a bi-cistronic construct; (x) co-expressed with a checkpoint inhibitor, optionally in separate constructs or in a bi-cistronic construct.

In those embodiments were the cell comprises one or more exogenous polynucleotides encoding an engager, the engager may comprise: (i) a first binding domain recognizing an extracellular portion of CD3, CD28, CD5, CD16, CD64, CD32, CD33, CD89, NKG2C, NKG2D, or any functional variants thereof of the cell or a by-stander immune effector cell; and (ii) a second binding domain targeting the second tumor antigen that is different from the first tumor antigen targeted by the exogenous TCR, and wherein the second binding domain of the engager is specific to any one of: B7H3, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2.

In some embodiments, the cell may further comprise one or more of: (i) CD38 knockout; (ii) HLA-I deficiency and/or HLA-II deficiency; (iii) introduced HLA-G or non-cleavable HLA-G, or knockout of one or both of CD58 and CD54; (iv) an exogenous CD16 or a variant thereof; (v) a chimeric fusion receptor (CFR); (vi) a signaling complex comprising a partial or full peptide of a cell surface expressed exogenous cytokine and/or a receptor thereof; (vii) at least one of the genotypes listed in Table 1; (viii) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (ix) introduction or upregulation of at least one of HLA-E, 4-1BBL, CD3, CD4, CD8, CD16, CD47, CD113, CD131, CD137, CD80, PDL1, A_{2A}R, Fc receptor, an antibody or functional variant or fragment thereof, a checkpoint inhibitor, and surface triggering receptor for coupling with an agonist. In those embodiments where the cell comprises an exogenous CD16 or a variant thereof, the exogenous CD16 or a variant thereof may comprise at least one of: (a) a high affinity non-cleavable CD16 (hnCD16); (b) F176V and S197P in ectodomain domain of CD16; (c) a full or partial ectodomain originated from CD64; (d) a non-native (or non-CD16) transmembrane domain; (e) a non-native (or non-CD16) intracellular domain; (f) a non-native (or non-CD16) signaling domain; (g) a non-native stimulatory domain; and (h) transmembrane, signaling, and stimulatory domains that are not originated from CD16, and are originated from a same or different polypeptide.

In those embodiments where the cell comprises a CFR, the CFR may comprise an ectodomain fused to a transmembrane domain, which is operatively connected to an endodomain, and wherein the ectodomain, transmembrane domain and the endodomain do not comprise any endoplasmic reticulum (ER) retention signals or endocytosis signals. In some embodiments, (i) the ectodomain of the CFR comprises a full or partial length of an extracellular portion of a signaling protein comprising at least one of CD3ε, CD3γ, CD3δ, CD28, CD5, CD16, CD64, CD32, CD33, CD89, NKG2C, NKG2D, any functional variants, and a combination or a chimera thereof; (ii) the ectodomain of the CFR initiates signal transduction upon binding to a selected agonist; or (iii) the endodomain of the CFR comprises a cytotoxicity domain comprising at least a full length or a portion of CD3ζ, 2B4, DAP10, DAP12, DNAM1, CD137 (4-1BB), IL21, IL7, IL12, IL15, NKp30, NKp44, NKp46, NKG2C, or NKG2D polypeptide; and optionally wherein the endodomain further comprises one or more of: (a) a co-stimulatory domain comprising a full length or a portion of CD2, CD27, CD28, CD40L, 4-1BB, OX40, ICOS, PD-1, LAG-3, 2B4, BTLA, DAP10, DAP12, CTLA-4, or NKG2D polypeptide, or any combination thereof; (b) a co-stimulatory domain comprising a full length or a portion of CD28, 4-1BB, CD27, CD40L, ICOS, CD2, or combinations thereof; (c) a persistency signaling domain comprising a full length or a portion of an endodomain of a cytokine receptor comprising IL7R, IL15R, IL18R, IL12R, IL23R, or combinations thereof; and/or (d) a full or a partial intracellular portion of a receptor tyrosine kinase (RTK), a tumor necrosis factor receptor (TNFR), an EGFR or a FAS receptor. In some embodiments, the selected agonist is (i) an antibody or a functional variant or fragment thereof; or (ii) an engager; and the selected agonist may be encoded by a polynucleotide comprised in the cell or is comprised in a medium comprising the cell or population thereof.

In those embodiments where the cell comprises a signaling complex comprising a partial or full peptide of a cell surface expressed exogenous cytokine and/or a receptor thereof, the cell surface expressed exogenous cytokine or receptor thereof may: (a) comprise at least one of IL2, IL4, IL6, IL7, IL9, IL10, IL11, IL12, IL15, IL18, IL21, and its respective receptor(s); or (b) comprise at least one of: (i) co-expression of IL15 and IL15Rα by using a self-cleaving peptide; (ii) a fusion protein of IL15 and IL15Rα; (iii) an IL15/IL15Rα fusion protein with intracellular domain of IL15Rα truncated or eliminated; (iv) a fusion protein of IL15 and membrane bound Sushi domain of IL15Rα; (v) a fusion protein of IL15 and IL15Rβ; (vi) a fusion protein of IL15 and common receptor γC, wherein the common receptor γC is native or modified; and (vii) a homodimer of IL15Rβ, wherein any one of (b)(i)-(vii) can be co-expressed with a CAR in separate constructs or in a bi-cistronic construct; or (c) comprise at least one of: (i) a fusion protein of IL7 and IL7Rα; (ii) a fusion protein of IL7 and common receptor γC, wherein the common receptor γC is native or modified; and (iii) a homodimer of IL7Rβ, wherein any one of (c)(i)-(iii) is optionally co-expressed with a CAR in separate constructs or in a bi-cistronic construct; and optionally, (d) be transiently expressed.

In those embodiments where the cell comprises a checkpoint inhibitor, the checkpoint inhibitor may be an antagonist to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, and an inhibitory KIR.

In another aspect, the invention provides a composition comprising the cell or population thereof as disclosed herein. In some embodiments of the composition, the cell or population thereof comprises the iPSC derivative effector cell, and wherein the composition further comprises one or more therapeutic agents. In some embodiments, the one or more therapeutic agents comprise a peptide, a cytokine, a checkpoint inhibitor, an antibody or functional variant or fragment thereof, an engager, a mitogen, a growth factor, a small RNA, a dsRNA (double stranded RNA), mononuclear blood cells, feeder cells, feeder cell components or replacement factors thereof, a vector comprising one or more polynucleic acids of interest, a chemotherapeutic agent or a radioactive moiety, or an immunomodulatory drug (IMiD). In those embodiments where the composition comprises a checkpoint inhibitor, the checkpoint inhibitor may comprise: (i) one or more antagonist checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or an inhibitory KIR; or (ii) one or more of atezolizumab, avelumab, durvalumab, ipilimumab, IPH4102, IPH43, IPH33, lirimumab, monalizumab, nivolumab, pembrolizumab, and their derivatives or functional equivalents. In some embodiments of the composition, the one or more therapeutic agents comprise one or more of venetoclax, azacitidine, and pomalidomide.

In those embodiments of the composition where the one or more therapeutic agents are an antibody, or functional variant or fragment thereof, the antibody, or functional variant or fragment thereof may comprise: (a) anti-CD20, anti-CD22, anti-HER2, anti-CD52, anti-EGFR, anti-CD123, anti-GD2, anti-PDL1, and/or anti-CD38 antibody; (b) one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or (c) daratumumab, and wherein the derivative effector cell comprises a CD38 knockout, and optionally expresses CD16 or a variant thereof. In those embodiments of the composition where the one or more therapeutic agents are an engager, the engager may comprise: (i) a bi-specific T cell engager (BiTE); (ii) a bi-specific killer cell engager (BiKE); or (iii) a tri-specific killer cell engager (TriKE); or the engager may comprise: (a) a first binding domain recognizing an extracellular portion of CD3, CD28, CD5, CD16, CD64, CD32, CD33, CD89, NKG2C, NKG2D, or any functional variants thereof of the cell or a bystander immune effector cell; and (b) a second binding domain specific to an antigen comprising any one of: B7H3, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2.

In yet another aspect, the invention provides for therapeutic use of the composition as provided herein by introducing the composition to a subject suitable for adoptive cell therapy, wherein the subject has an autoimmune disorder, a hematological malignancy, a solid tumor, cancer, or a viral infection.

In yet another aspect, the invention provides a master cell bank (MCB) comprising the clonal iPSC provided herein.

In yet another aspect, the invention provides a method of manufacturing a derivative effector cell described herein, where the method comprises: differentiating a genetically engineered iPSC, wherein the iPSC comprises: (a) a polynucleotide encoding a transgenic TCRα chain (tgTCRα); (b) a polynucleotide encoding a transgenic TCRβ chain (tgTCRβ); and optionally, (c) a polynucleotide encoding a chimeric antigen receptor (CAR) or an engager targeting a second tumor antigen; and optionally wherein the iPSC further comprises one or more of: (i) CD38 knockout; (ii) HLA-I deficiency and/or HLA-II deficiency; (iii) introduced HLA-G or non-cleavable HLA-G, or knockout of one or both of CD58 and CD54; (iv) an exogenous CD16 or a variant thereof; (v) a chimeric fusion receptor (CFR); (vi) a signaling complex comprising a partial or full peptide of a cell surface expressed exogenous cytokine and/or a receptor thereof; (vii) at least one of the genotypes listed in Table 1; (viii) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (ix) introduction or upregulation of at least one of HLA-E, 4-1BBL, CD3, CD4, CD8, CD16, CD47, CD113, CD131, CD137, CD80, PDL1, A_{2A}R, Fc receptor, an antibody or functional variant or fragment thereof, a checkpoint inhibitor, and surface triggering receptor for coupling with an agonist. In some embodiments, the method further comprises: genomically engineering a clonal iPSC to knock in: (a) the polynucleotide encoding the transgenic TCRα chain (tgTCRα); (b) the polynucleotide encoding the transgenic TCRβ chain (tgTCRβ); and optionally, (c) the polynucleotide encoding the chimeric antigen receptor (CAR) or the engager targeting the second tumor antigen; and optionally further comprising genomically engineering the clonal iPSC: (i) to knock out CD38, (ii) to knock out B2M and/or CIITA, (iii) to knock out one or both of CD58 and CD54, and/or (iv) to introduce HLA-G or non-cleavable HLA-G, the exogenous CD16 or a variant thereof, the CFR, and/or the signaling complex comprising the partial or full peptide of the cell surface expressed exogenous cytokine and/or receptor thereof. In some embodiments, the genomic engineering comprises targeted editing. In particular embodiments, the targeted editing comprises deletion, insertion, or in/del, and wherein the targeted editing is carried out by CRISPR, ZFN, TALEN, homing nuclease, homology recombination, or any other functional variation of these methods.

In yet another aspect, the invention provides a chimeric antigen receptor (CAR) specific to tumor cell surface antigen MR1, wherein the MR1-CAR comprises: (i) an ectodomain comprising at least one antigen recognition domain, wherein the antigen recognition domain comprises: (a) a variable alpha (Vα) fragment that has at least about 85% identity to SEQ ID NO: 7 (MR1Vα), and a variable beta (Vβ) fragment that has at least about 85% identity to SEQ ID NO: 8 (MR1Vβ); or (b) an extracellular domain of MR1 TCRα that has at least about 85% identity to SEQ ID NO: 15, and an extracellular domain of MR1 TCRβ that has at least about 85% identity to SEQ ID NO: 16; (ii) a transmembrane domain; and (iii) an endodomain comprising at least a first signaling domain, wherein the first signaling domain is originated from a cytoplasmic domain of a signal transducing protein specific to T and/or NK cell activation or functioning; and wherein the tumor cell surface antigen MR1 is non-polymorphic. In some embodiments, the signal transducing protein comprises any one of: 2B4 (Natural killer Cell Receptor 2B4), 4-1BB (Tumor necrosis factor receptor superfamily member 9), CD16 (IgG Fc region Receptor III-A), CD2 (T cell surface antigen CD2), CD28 (T cell-specific surface glycoprotein CD28), CD28H (Transmembrane and immunoglobulin domain-containing protein 2), CD3ζ (T cell surface glycoprotein CD3 zeta chain), CD3 ζ1XX (CD3ζ variant), DAP10 (Hematopoietic cell signal transducer), DAP12 (TYRO protein tyrosine kinase-binding protein), DNAM1 (CD226 antigen), FcERIγ (High affinity immunoglobulin epsilon receptor subunit gamma), IL21R (Interleukin-21 receptor), IL-2Rβ/IL-15RB (Interleukin-2 receptor subunit beta), IL-2Rγ (Cytokine receptor common subunit gamma), IL-7R (Interleukin-7 receptor subunit alpha), KIR2DS2 (Killer cell immunoglobulin-like receptor 2DS2), NKG2D (NKG2-D type II integral membrane protein), NKp30 (Natural cytotoxicity triggering receptor 3), NKp44 (Natural cytotoxicity triggering receptor 2), NKp46 (Natural cytotoxicity triggering receptor 1), CS1 (SLAM family member 7), and CD8 (T cell surface glycoprotein CD8 alpha chain).

In some embodiments of the CAR, the endodomain further comprises a second signaling domain, and optionally a third signaling domain; and wherein the first, second and third signaling domains are different. In some embodiments, the second or the third signaling domain comprises a cytoplasmic domain, or a portion thereof, of 2B4, 4-1BB, CD16, CD2, CD28, CD28H, CD3ζ, DAP10, DAP12, DNAM1, FcERIγ IL21R, (IL-15Rβ), IL-2Rγ, IL-7R, KIR2DS2, NKG2D, NKp30, NKp44, NKp46, CD3 ζ1XX, CS1, or CD8. In some embodiments, the transmembrane domain comprises an amino acid sequence of a transmembrane region, or a portion thereof, of CD2, CD3δ, CD3ε, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide. In some embodiments, the ectodomain further comprises: (i) a signal peptide; and/or (ii) a spacer/hinge/linker. In some embodiments, the CAR is comprised in a bi-cistronic construct co-expressing a partial or full length peptide of a cell surface expressed exogenous cytokine or a receptor thereof, wherein the exogenous cytokine or receptor thereof: (a) comprises at least one of IL2, IL4, IL6, IL7, IL9, IL10, IL11, IL12, IL15, IL18, IL21, and its respective receptor(s); or (b) comprises at least one of: (i) co-expression of IL15 and IL15Rα by using a self-cleaving peptide; (ii) a fusion protein of IL15 and IL15Rα; (iii) an IL15/IL15Rα fusion protein with intracellular domain of IL15Rα truncated or eliminated; (iv) a fusion protein of IL15 and membrane bound Sushi domain of IL15Rα; (v) a fusion protein of IL15 and IL15Rβ; (vi) a fusion protein of IL15 and common receptor γC, wherein the common receptor γC is native or modified; and (vii) a homodimer of IL15Rβ, wherein any one of (b)(i)-(vii) is optionally co-expressed with a CAR in separate constructs or in a bi-cistronic construct; or (c) comprises at least one of: (i) a fusion protein of IL7 and IL7Rα; (ii) a fusion protein of IL7 and common receptor γC, wherein the common receptor γC is native or modified; and (iii) a homodimer of IL7Rβ. In particular embodiments, the MR1-CAR is specific for one or more of colorectal cancer, lung cancer, kidney cancer, prostrate cancer, bladder cancer, cervical cancer, melanoma, bone cancer, breast cancer, ovarian cancer or blood cancer.

In yet another aspect, the invention provides a cell or a population thereof, wherein the cell is an eukaryotic cell, an animal cell, a human cell, an immune cell, an induced pluripotent cell (iPSC), a clonal iPSC or a derivative cell differentiated therefrom, and wherein the cell comprises a polynucleotide encoding at least the chimeric antigen receptor (CAR) as described herein. In various embodiments of the cell or a population thereof, the cell further comprises (i) a polynucleotide encoding a transgenic TCRα chain (tgTCRα); and (ii) a polynucleotide encoding a transgenic TCRβ chain (tgTCRβ), wherein the tgTCRα chain and the tgTCRβ chain form an exogenous TCR complex (TCR^{exo}) that recognizes a first tumor antigen other than MR1; and optionally, (iii) one or more additional exogenous polynucleotides comprising a polynucleotide encoding an engager targeting at least a second tumor antigen. In various embodiments, (i) the polynucleotide encoding the tgTCRα chain and the polynucleotide encoding the tgTCRβ chain are comprised in a bi-cistronic construct, and optionally wherein: (a) the construct is inserted at a constant region of TCRα or TCRβ (TRAC or TRBC); (b) the insertion of the construct disrupts expression of endogenous TCRα or TCRβ at the insertion site; and/or (c) expression of the construct is driven by an endogenous promoter of TCR or an exogenous promoter; or (ii) the polynucleotide encoding the CAR or the engager is inserted at TRAC or TRBC, and optionally wherein: (a) the insertion of the polynucleotide encoding the CAR or the engager disrupts expression of the endogenous TCRα or TCRβ at the insertion site; and/or (b) expression of the CAR or the engager is driven by an endogenous promoter of TCR or an exogenous promoter; or (iii) the polynucleotides encoding the tgTCRα chain and the tgTCRβ chain, the CAR or the engager, or the one or more additional polynucleotides are inserted in one, or more safe harbor loci or selected gene loci. In particular embodiments, (I) wherein the construct and the polynucleotide encoding the CAR or the engager are each inserted at a constant region of TCRα or TCRβ (TRAC or TRBC), but not at the same constant region, thereby disrupting expression of both endogenous TCRα and TCRβ, knocking out the endogenous TCR, and avoiding a mispaired TCR comprising: (a) the transgenic TCRα and the endogenous TCRβ, or (b) the transgenic TCRβ and the endogenous TCRα; or (II) wherein the construct and the polynucleotide encoding the CAR or the engager are each integrated at a locus comprising a safe harbor locus or a selected gene locus.

In yet another aspect, the invention provides a composition comprising the cell or a population thereof described herein. In some embodiments, the cell or population thereof comprises the iPSC derivative effector cell, and wherein the composition further comprises one or more therapeutic agents. Thus, embodiments of the invention also provide for therapeutic use of a composition described herein by introducing the composition to a subject suitable for adoptive cell therapy, wherein the subject has an autoimmune disorder, a hematological malignancy, a solid tumor, cancer, or a viral infection. In some embodiments, the subject has colorectal cancer, lung cancer, kidney cancer, prostrate cancer, bladder cancer, cervical cancer, stomach cancer, melanoma, bone cancer, breast cancer, ovarian cancer or blood cancer.

In yet another aspect, the invention provides a method of enhancing a CAR-T cell function, wherein the CAR-T cell has a first tumor antigen specificity via a chimeric antigen receptor (CAR), and wherein the method comprises introducing to the CAR-T cell: (i) a polynucleotide encoding a transgenic TCRα chain (tgTCRα); and (ii) a polynucleotide encoding a transgenic TCRβ chain (tgTCRβ), wherein the tgTCRα chain and the tgTCRβ chain form an exogenous TCR complex (TCR^{exo}) that has a second tumor antigen specificity; wherein the CAR-induced tumor killing efficacy of the CAR-T cell is enhanced by expression of TCR^{exo}. In various embodiments of the method, (i) an endogenous TCR knockout by disrupting expression of both endogenous TCRα and TCRβ; or (ii) the CAR is inserted at a constant region of TCRα or TCRβ (TRAC or TRBC), thereby disrupting expression of endogenous TCRα or TCRβ of the VAR-T cell. In various embodiments of the method, the method further comprises activating TCR^{exo} using the second tumor antigen recognized by TCR^{exo}, wherein the first tumor antigen specificity and the second tumor antigen specificity are different. In various embodiments of the method, the stop of introducing to the CAR-T cell a polynucleotide further comprises: differentiating a genetically engineered iPSC to a T cell, wherein the iPSC comprises: (a) the polynucleotide encoding a transgenic TCRα chain (tgTCRα); (b) the polynucleotide encoding a transgenic TCRβ chain (tgTCRβ); and (c) a polynucleotide encoding the CAR having the first tumor antigen specificity, thereby obtaining the CAR-T cell having expression of the TCR^{exo}. In various embodiments of the method, the method further comprises genomically engineering a clonal iPSC to knock in: (a) the polynucleotide encoding the transgenic TCRα chain (tgTCRα); (b) the polynucleotide encoding the transgenic TCRβ chain (tgTCRβ); and (c) the polynucleotide encoding the CAR having the first tumor antigen specificity, thereby obtaining an engineered iPSC for T cell differentiation.

Various objects and advantages of the compositions and methods as provided herein will become apparent from the following description taken in conjunction with the accompanying drawings wherein are set forth, by way of illustration and example, certain embodiments of this invention.

### BRIEF DESCRIPTION OF THE DRAWINGS

FIGS. 1A and 1B show ectopic expression of MR1-TCR in iPSC-derived effector cells. FIG. 1A shows transduction of an MR1-TCR construct into iPSC reprogrammed from T cells (TiPSC) or from fibroblasts (FiPSC) and subsequent differentiation thereof into T lineage effector cells (TiP-iT or FiP-iT, respectively). FIG. 1B shows that the presence of endogenous TCRβ in TiPSC can lead to TCR mispairing in derivative T lineage effector cells (TiP-iT) that have endogenous TCR knockout through TRAC disruption.

FIG. 2 shows that expression of TCRαβ can stabilize surface expression of CD3 molecules.

FIG. 3 shows that MR1 TCR-expressing effector cells can specifically kill A549 tumor cells, which can be inhibited by an MR1 blocking antibody.

FIGS. 4A and 4B show that both FiPSC- (FIG. 4A) and TiPSC− (FIG. 4B) derived MR1-TCR⁺ iT cells demonstrate MR1-dependent cytokine release and degranulation responses.

FIGS. 5A and 5B show the purity of FiP-iT cells expressing MR1-TCR within the input population (FIG. 5A), and that MR1 TCR⁺ FiP-iT demonstrated cytotoxicity in an MR1-dependent manner with A549 cells (FIG. 5B), and that MR1-mediated TCR signaling blocked by an antibody can be restored by the addition of BiTE.

FIG. 6 shows that an MR1 blocking antibody can prevent MR1-TCR mediated killing of tumor cells (top), and that addition of BiTE allows targeting of the tumor cells via MR1-TCR reconstituted cell surface CD3 engagement even in the presence of the antibody.

FIGS. 7A and 7B show that BiTE restored cell activation and response in MR1-TCR⁺ FiP-iT cells in the presence of MR1 blocking antibody.

FIGS. 8A and 8B show that BiTE restored cell activation and response in MR1-TCR⁺ TiP-iT cells when the MR1 recognition by the TCR is blocked.

FIGS. 9A and 9B show that MR1-TCR⁺ FiP-iT demonstrated cytotoxicity in an MR1-dependent manner with Nalm6 cells, and that MR1-mediated TCR signaling and cytotoxicity blocked by an antibody can be restored by the addition of BiTE.

FIG. 10 shows exemplary MR1-CAR designs where the transmembrane (TM) domain and cytoplasmic signaling domain can vary.

FIGS. 11A-11E show that TRBC knockout reduces mispairing issues and restores TCR function. In FIG. 11D, the x-axis for % TNFα is 0.0, 0.5, 1.0, 1.5, and 2.0, from left to right.

FIGS. 12A-12D show a synergistic relationship between CAR and TCR co-expression on TiP-iT cells.

FIGS. 13A and 13B demonstrate in vitro cytotoxicity of TiP-iT cells having CAR, TCR and CD16 co-expression (tri-modal).

FIGS. 14A-14G show additional data supporting the synergistic relationship between CAR and TCR co-expression on the tri-modal TiP-iT cells, and demonstrate in vitro cytotoxicity of the tri-modal cells.

FIG. 15 shows that the exogenous TCR expressed in iT effectors is capable of triggering TCR antigen-specific tumor killing.

FIG. 16 shows that the exogenous expression of TCR enhances CAR-induced tumor killing efficacy in iT effectors.

FIG. 17 shows that in the presence of both CAR and TCR tumor antigen, CAR/TCR iT effectors show enhanced tumor killing efficacy compared to CAR iT.

FIG. 18 shows that activation of both TCR and CAR leads to faster tumor killing compared to CAR activation alone.

FIG. 19 shows that the exogenous TCR expression in CAR iT cells correlates higher iT effector counts in cell expansion.

FIG. 20A shows Day 0 mixed tumor cell lines each of which triggers activation of CAR, TCR, or ADCC mediated by IgG antibody that binds to CD16. FIG. 20B illustrates the scheme of in vivo validation of tri-modal iT cells using Nalm6 mouse model.

FIG. 21A shows the FACS analysis of ex vivo CD10⁺ cells from bone marrow for BCMA-mCherry and NYESO1-GFP signals. FIG. 21B shows the absolute tumor count in each bone marrow sample for indicated tumor lines in all groups as indicated.

FIG. 22 shows that CAR/TCR iT cells cleared majority of tumors in bone marrow on day 7 and 10 but not in lung and brain area.

FIG. 23A shows representative FACS plot showing the ratio between CAR-targeted Nalm6 (BCMA⁺) and TCR-targeted Nalm6 (BCMA⁻) of input, and the analysis of day 22 bone marrow cell suspension from no effector, BCMA CAR alone, BCMA-CAR⁺NYESO1-TCR⁺ and BCMA-CAR⁺MR1-TCR⁺ groups. FIG. 23B presents the absolute target count of BCMA⁺MR1^{ko} and NYESO1⁺MR1^{wt} Nalm6 for individual groups as indicated.

FIGS. 24A and 24B provide confirmation of improved tumor growth inhibition (TGI) in mice treated with CAR/TCR iT cells using (FIG. 24A) BLI and (FIG. 24B) Area Under Curve (AUC) analyses.

FIG. 25 shows a representative FACS plot demonstrating the absence of checkpoint inhibitory receptor, LAG3, TIM3, PD1 on ex vivo iT cells of each indicated group from bone marrow on day 22.

FIG. 26 demonstrates the cytokine production and degranulation capacity of ex vivo iT cells of each indicated group from bone marrow on day 22.

### DETAILED DESCRIPTION OF THE INVENTION

Genomic modification of iPSCs (induced pluripotent stem cells) includes polynucleotide insertion, deletion and substitution. Exogenous gene expression in genome-engineered iPSCs often encounters problems such as gene silencing or reduced gene expression after prolonged clonal expansion of the original genome-engineered iPSCs, after cell differentiation, and in dedifferentiated cell types from the cells derived from the genome-engineered iPSCs. On the other hand, direct engineering of primary immune cells such as T or NK cells is challenging, and presents a hurdle to the preparation and delivery of engineered immune cells for adoptive cell therapy. In various embodiments, the present invention provides an efficient, reliable, and targeted approach for stably integrating one or more exogenous genes, including suicide genes and other functional modalities, which provide improved therapeutic properties relating to engraftment, trafficking, homing, migration, cytotoxicity, viability, maintenance, expansion, longevity, self-renewal, persistence, and/or survival, into iPSC derivative cells, including but not limited to HSCs (hematopoietic stem and progenitor cell), T cell progenitor cells, NK cell progenitor cells, T lineage cells, NKT lineage cells, NK lineage cells, and immune effector cells having one or more functional features that are not present in primary NK, T, and/or NKT cells.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition at a functionally inert, low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” refers to the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs” refers to stem cells that are produced in vitro, using reprogramming factor and/or small molecule chemical driven methods, from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta, i.e., are not totipotent.

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “naïve” or “ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed-state of pluripotency. Under standard pluripotent cell culture conditions such cells remain in the primed state unless the exogenous transgene expression is maintained, wherein characteristics of the ground-state are observed.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical inter-cell spacing.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate” or “maintain” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

As used herein, the term “mesoderm” refers to one of the three germinal layers that appears during early embryogenesis and which gives rise to various specialized cell types including blood cells of the circulatory system, muscles, the heart, the dermis, skeleton, and other supportive and connective tissues.

As used herein, the term “definitive hemogenic endothelium” (HE) or “pluripotent stem cell-derived definitive hemogenic endothelium” (iHE) refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T lineage cells, NK lineage cells and B lineage cells. Hematopoietic cells also include various subsets of primitive hematopoietic cells that give rise to primitive erythrocytes, megakarocytes and macrophages.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells in an MHC class I-restricted manner. A T cell can be any T cell, such as a cultured T cell, e.g., a primary T cell, or a T cell from a cultured T cell line, e.g., Jurkat, SupT1, etc., or a T cell obtained from a mammal. The T cell can be a CD3⁺ cell. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The T cell can also refer to a genetically engineered T cell, such as a T cell modified to express a T cell receptor (TCR) or a chimeric antigen receptor (CAR). A T cell, or a T cell like effector cell can also be differentiated from a stem cell or progenitor cell. A T cell like derivative effector cell may have a T cell lineage in some respects, but at the same time has one or more functional features that are not present in a primary T cell.

“CD4⁺ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” molecules are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRγ, and EAT-2. In some embodiments, isolated subpopulations of CD56⁺ NK cells comprise expression of CD16, NKG2C, CD57, NKG2D, NCR ligands, NKp30, NKp40, NKp46, activating and inhibitory KIRs, NKG2A and/or DNAM-1. CD56⁺ can be dim or bright expression. An NK cell, or an NK cell like effector cell may be differentiated from a stem cell or progenitor cell. An NK cell like derivative effector cell may have an NK cell lineage in some respects, but at the same time has one or more functional features that are not present in a primary NK cell.

As used herein, the term “NKT cells” or “natural killer T cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCR αβ usage. Type I NKT cells are considered suitable for immunotherapy. Adaptive or invariant (type I) NKT cells can be identified with the expression of at least one or more of the following markers, TCR Va24-Ja18, Vb11, CD1d, CD3, CD4, CD8, aGalCer, CD161 and CD56.

The term “effector cell” generally is applied to certain cells in the immune system that carry out a specific activity in response to stimulation and/or activation, or to cells in the nervous system that effect a specific function upon activation. As used herein, the term “effector cell” is interchangeable with “differentiated immune cell,” which refer to cells that are edited and/or modulated to carry out a specific activity in response to stimulation and/or activation. Non-limiting examples of effector cells include T cells, NK cells, NKT cells, B cells, macrophages, and neutrophils.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an isolated cell is partly or completely separated from at least one component, including other substances, cells or cell populations, as it is found in nature or as it is grown, stored or subsisted in non-naturally occurring environments. Specific examples of isolated cells include partially pure cell compositions, substantially pure cell compositions and cells cultured in a medium that is non-naturally occurring. Isolated cells may be obtained by separating the desired cells, or populations thereof, from other substances or cells in the environment, or by removing one or more other cell populations or subpopulations from the environment.

As used herein, the term “purify” or the like refers to increasing purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof a variant polypeptide (i.e., a mutant of the native polypeptide having less than 100% sequence identity with the native polypeptide) or fragment thereof an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “subunit” as used herein refers to each separate polypeptide chain of a protein complex, where each separate polypeptide chain can form a stable folded structure by itself. Many protein molecules are composed of more than one subunit, where the amino acid sequences can either be identical for each subunit, or similar, or completely different. For example, a CD3 complex is composed of CD3α, CD3ε, CD3δ, CD3γ, and CD3ζ subunits, which form the CD3ε/CD3γ, CDR/CD3δ, and CD3ζ/CD3ζ dimers. Within a single subunit, contiguous portions of the polypeptide chain frequently fold into compact, local, semi-independent units that are called “domains”. Many protein domains may further comprise independent “structural subunits”, also called subdomains, contributing to a common function of the domain. As such, the term “subdomain” as used herein refers to a protein domain inside of a larger domain, for example, a binding domain within an ectodomain of a cell surface receptor; or a stimulatory domain or a signaling domain of an endodomain of a cell surface receptor.

“Operably-linked” or “operatively linked,” interchangeable with “operably connected” or “operatively connected,” refers to the association of nucleic acid sequences on a single nucleic acid fragment (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation. As a further example, a receptor-binding domain can be operatively connected to an intracellular signaling domain, such that binding of the receptor to a ligand transduces a signal responsive to said binding.

“Fusion proteins” or “chimeric proteins”, as used herein, are proteins created through genetic engineering to join two or more partial or whole polynucleotide coding sequences encoding separate proteins, and the expression of these joined polynucleotides results in a single peptide or multiple polypeptides with functional properties derived from each of the original proteins or fragments thereof. Between two neighboring polypeptides of different sources in the fusion protein, a linker (or spacer) peptide can be added. The chimeric fusion receptors (CFRs) described herein are fusion, or chimeric, proteins.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK lineage cells or derivative T lineage cells, as used throughout this application are cells differentiated from an iPSC, as compared to their counterpart primary cells obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing. In the aspect of a source cell obtained from a specifically selected donor, disease or treatment context, the genetic imprint contributing to preferential therapeutic attributes may include any context specific genetic or epigenetic modifications which manifest a retainable phenotype, i.e., a preferential therapeutic attribute, that is passed on to iPSC-derived cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include, but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements, i.e., selected HLA-matched donor cells exhibiting a haplotype with increased population. As used herein, preferential therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy. When derivative cells having one or more therapeutic attributes are obtained from differentiating an iPSC that has genetic imprint(s) conferring a preferential therapeutic attribute incorporated thereto, such derivative cells are also called “synthetic cells”. In general, a synthetic cell possesses one or more non-native cell functions when compared to its closest counterpart primary cell, whether the synthetic cell is differentiated from engineered pluripotent cells or obtained by engineering a primary cell from natural/native sources, such as peripheral blood, umbilical cord blood, or other donor tissues.

The term “enhanced therapeutic property” as used herein, refers to a therapeutic property of a cell that is enhanced as compared to a typical immune cell of the same general cell type. For example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy.

As used herein, the term “engager” refers to a molecule, e.g., a fusion polypeptide, which is capable of forming a link between an immune cell (e.g., a T cell, a NK cell, a NKT cell, a B cell, a macrophage, or a neutrophil), and a tumor cell; and activating the immune cell. Examples of engagers include, but are not limited to, bi-specific T cell engagers (BiTEs), bi-specific killer cell engagers (BiKEs), tri-specific killer cell engagers (TriKEs), or multi-specific killer cell engagers, and universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, a NK cell, a NKT cell, a B cell, a macrophage, or a neutrophil. In some embodiments, the surface triggering receptor facilitates bi- or multi-specific antibody engagement between the effector cells and specific target cell (e.g., a tumor cell) independent of the effector cell's natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some case, and to kill two or more types of tumors in some other cases. A surface triggering receptor generally comprises a co-stimulatory domain for effector cell activation and an epitope that is specific to the epitope binding region of an engager. A bi-specific engager is specific to the epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis, DNA replication, growth arrest, transcriptional and post-transcriptional genetic regulation and/or antibody-mediated depletion. In some instances, the safety switch protein is activated by an exogenous molecule, e.g., a prodrug, that when activated, triggers apoptosis and/or cell death of a therapeutic cell. Examples of safety switch proteins include, but are not limited to, suicide genes such as caspase 9 (or caspase 3 or 7), thymidine kinase, cytosine deaminase, B cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refers to proteins or peptides that are capable of achieving a biological and/or pharmaceutical effect on an organism. A pharmaceutically active protein has healing, curative or palliative properties against a disease and may be administered to ameliorate relieve, alleviate, reverse or lessen the severity of a disease. A pharmaceutically active protein also has prophylactic properties and is used to prevent the onset of a disease or to lessen the severity of such disease or pathological condition when it does emerge. Pharmaceutically active proteins include an entire protein or peptide, pharmaceutically active fragments or variants of the protein or peptide, pharmaceutically active analogs of the protein or peptide, and analogs of fragments of the protein or peptide. The term “pharmaceutically active protein” also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, cellular signal transduction. “Signal transduction” refers to the transmission of a molecular signal in the form of chemical modification by recruitment of protein complexes along a pathway that ultimately triggers a biochemical event in the cell. Signal transduction pathways are well known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes but is not limited to i) antigen specificity as it relates to a unique chimeric antigen receptor (CAR) or T cell receptor (TCR), ii) engager specificity as it relates to monoclonal antibodies or bi-specific engagers, iii) targeting of transformed cells, iv) targeting of cancer stem cells, and v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

The term “adoptive cell therapy” as used herein refers to a cell-based immunotherapy that relates to the transfusion of autologous or allogenic lymphocytes, identified as T or B cells, genetically modified or not, that have been expanded ex vivo prior to said transfusion.

A “therapeutically sufficient amount”, as used herein, includes within its meaning a non-toxic, but sufficient and/or effective amount of the particular therapeutic and/or pharmaceutical composition to which it is referring to provide a desired therapeutic effect. The exact amount required will vary from subject to subject depending on factors such as the patient's general health, the patient's age and the stage and severity of the condition. In particular embodiments, a “therapeutically sufficient amount” is sufficient and/or effective to ameliorate, reduce, and/or improve at least one symptom associated with a disease or condition of the subject being treated.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate the lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (i.e., ectoderm, mesoderm and endoderm germ layers) with modest proliferation within the EB cell cluster. Although proven to facilitate cell differentiation, EBs, however, give rise to heterogeneous cells in variable differentiation states because of the inconsistent exposure of the cells in the three-dimensional structure to differentiation cues from the environment. In addition, EBs are laborious to create and maintain. Moreover, cell differentiation through EB formation is accompanied with modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates forming larger aggregates, these aggregates can be routinely mechanically or enzymatically dissociated into smaller aggregates to maintain cell proliferation within the culture and increase numbers of cells. As distinct from EB culture, cells cultured within aggregates in maintenance culture maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells, i.e., “EB formation.” Monolayer differentiation, among other advantages disclosed herein, avoids the need for EB formation to initiate differentiation. Because monolayer culturing does not mimic embryo development such as EB formation, differentiation towards specific lineages is deemed as minimal as compared to all three germ layer differentiation in EB.

As used herein, a “dissociated cell” or “single dissociated cell” refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be enzymatically or mechanically dissociated from each other, such as by dissociation into a suspension of clusters, single cells or a mixture of single cells and clusters. In yet another alternative embodiment, adherent cells can be dissociated from a culture plate or other surface. Dissociation thus can involve breaking cell interactions with extracellular matrix (ECM) and substrates (e.g., culture surfaces), or breaking the ECM between cells.

As used herein, a “master cell bank” or “MCB” refers to a clonal master engineered iPSC line, which is a clonal population of iPSCs that have been engineered to comprise one or more therapeutic attributes, have been characterized, tested, qualified, and expanded, and have been shown to reliably serve as the starting cellular material for the production of cell-based therapeutics through directed differentiation in manufacturing settings. In various embodiments, an MCB is maintained, stored, and/or cryopreserved in multiple vessels to prevent genetic variation and/or potential contamination by reducing and/or eliminating the total number of times the iPS cell line is passaged, thawed or handled during the manufacturing processes.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an antagonistic mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day, and therefore contains many mediator substances, including growth factors and cytokines secreted by the feeder cells cultivated in the medium. In some embodiments, a feeder-free environment is free of both feeder or stromal cells and is also not pre-conditioned by the cultivation of feeder cells.

“Functional” as used in the context of genomic editing or modification of iPSCs, and derived non-pluripotent cells differentiated therefrom, or genomic editing or modification of non-pluripotent cells and derived iPSCs reprogrammed therefrom, refers to (1) at the gene level—successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level—successful removal, addition, or alteration of a cell function/characteristic via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

“HLA deficient”, including HLA class I deficient, or HLA class II deficient, or both, refers to cells that either lack, or no longer maintain, or have a reduced level of surface expression of a complete MHC complex comprising an HLA class I protein heterodimer and/or an HLA class II heterodimer, such that the diminished or reduced level is less than the level naturally detectable by other cells or by synthetic methods.

“Modified HLA deficient iPSC,” as used herein, refers to an HLA deficient iPSC that is further modified by introducing genes expressing proteins related, but not limited to improved differentiation potential, antigen targeting, antigen presentation, antibody recognition, persistence, immune evasion, resistance to suppression, proliferation, co-stimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL11b, NOTCH, RUNX1, IL15, 4-1BB, DAP10, DAP12, CD24, CD3ζ, 4-1BBL, CD47, CD113, and PDL1. The cells that are “modified HLA deficient” also include cells other than iPSCs.

The term “ligand” refers to a substance that forms a complex with a target molecule to produce a signal by binding to a site on the target. The ligand may be a natural or artificial substance capable of specific binding to the target. The ligand may be in the form of a protein, a peptide, an antibody, an antibody complex, a conjugate, a nucleic acid, a lipid, a polysaccharide, a monosaccharide, a small molecule, a nanoparticle, an ion, a neurotransmitter, or any other molecular entity capable of specific binding to a target. The target to which the ligand binds, may be a protein, a nucleic acid, an antigen, a receptor, a protein complex, or a cell. A ligand that binds to and alters the function of the target and triggers a signaling response is called “agonistic” or “an agonist”. A ligand that binds to a target and blocks or reduces a signaling response is “antagonistic” or “an antagonist.”

The term “antibody” encompasses antibodies and antibody fragments that contain at least one binding site that specifically binds to a particular target of interest, wherein the target may be an antigen, or a receptor that is capable of interacting with certain antibodies. The term “antibody” includes, but is not limited to, an immunoglobulin molecule or an antigen-binding or receptor-binding portion thereof. For example, an NK cell can be activated by the binding of an antibody or the Fc region of an antibody to its Fc-gamma receptors (FcγR), thereby triggering the ADCC (antibody-dependent cellular cytotoxicity) mediated effector cell activation. A specific piece or portion of an antigen or receptor, or a target in general, to which an antibody binds is known as an epitope or an antigenic determinant. The term antibody also includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. An antibody may be a murine antibody, a human antibody, a humanized antibody, a camel IgG, single variable new antigen receptor (VNAR), shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody.

“Fc receptors,” abbreviated FcR, are classified based on the type of antibody that they recognize. For example, those that bind the most common class of antibody, IgG, are called Fc-gamma receptors (FcγR), those that bind IgA are called Fc-alpha receptors (FcαR) and those that bind IgE are called Fc-epsilon receptors (FcεR). The classes of FcRs are also distinguished by the cells that express them (macrophages, granulocytes, natural killer cells, T and B cells) and the signaling properties of each receptor. Fc-gamma receptors (FcγR) include several members, FcγRI (CD64), FcγRIIA (CD32), FcγRIIB (CD32), FcγRIIIA (CD16a), and FcγRIIIB (CD16b), which differ in their antibody affinities due to their different molecular structures.

“Chimeric Receptor” is a general term used to describe an engineered, artificial, or hybrid receptor protein molecule that is made to comprise two or more portions of amino acid sequences that are originated from at least two different proteins. The chimeric receptor proteins have been engineered to give a cell the ability to initiate signal transduction and carry out downstream function upon binding of an agonistic ligand to the receptor. Exemplary “chimeric receptors” include, but are not limited to, chimeric antigen receptors (CARs), chimeric fusion receptors (CFRs), chimeric Fc receptors (CFcRs), fusions of two or more receptors, as well as recombinant TCRs (rTCR, or exogenous TCR) having specificity to a tumor associated antigen (TAA).

“Chimeric Fc Receptor,” abbreviated as “CFcR,” is a term used to describe engineered Fc receptors having their native transmembrane and/or intracellular signaling domains modified or replaced with non-native transmembrane and/or intracellular signaling domains. In some embodiments of the chimeric Fc receptor, in addition to having one of, or both of, the transmembrane and signaling domains being non-native, one or more stimulatory domains can be introduced to the intracellular portion of the engineered Fc receptor to enhance cell activation, expansion and function upon triggering of the receptor. Unlike a chimeric antigen receptor (CAR), which contains an antigen binding domain to a target antigen, the chimeric Fc receptor binds to an Fc fragment, or the Fc region of an antibody, or the Fc region comprised in an engager or a binding molecule and activates the cell function with or without bringing the targeted cell close in vicinity. For example, a Fcγ receptor can be engineered to comprise selected transmembrane, stimulatory, and/or signaling domains in the intracellular region that respond to the binding of IgG at the extracellular domain, thereby generating a CFcR. In one example, a CFcR is produced by engineering CD16, a Fcγ receptor, by replacing its transmembrane domain and/or intracellular domain. To further improve the binding affinity of the CD16-based CFcR, the extracellular domain of CD64 or the high-affinity variants of CD16 (F176V, for example) can be incorporated. In some embodiments of the CFcR where a high affinity CD16 extracellular domain is involved, the proteolytic cleavage site comprising a serine at position 197 is eliminated or is replaced such that the extracellular domain of the receptor is non-cleavable, i.e., not subject to shedding, thereby obtaining a hnCD16-based CFcR.

CD16, a FcγR receptor, has been identified to have two isoforms, Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16” (abbreviated as hnCD16), as used herein, refers to a natural or non-natural variant of CD16. The wildtype CD16 has low affinity and is subject to ectodomain shedding, a proteolytic cleavage process that regulates the cell surface density of various cell surface molecules on leukocytes upon NK cell activation. F176V and F158V are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in WO2015/148926, the complete disclosure of which is incorporated herein by reference. The CD16 S197P variant is an engineered non-cleavable version of CD16. A CD16 variant comprising both F158V and S197P has high affinity and is non-cleavable. Another exemplary high affinity and non-cleavable CD16 (hnCD16) variant is an engineered CD16 comprising an ectodomain originated from one or more of the 3 exons of the CD64 ectodomain.

“T cell receptor,” abbreviated as “TCR,” generally refers to a protein complex found on the surface of a T cell and is responsible for recognizing fragments of antigen peptides bound to major histocompatibility complex (MHC) molecules. Binding of a TCR to an antigen peptide initiates TCR-CD3 intracellular activation, recruitment of numerous signaling molecules, and branching and integrating signaling pathways, leading to mobilization of transcription factors that are critical for gene expression and T cell growth and function acquisition. A typical TCR comprises two highly variable protein chains (α and β), with each chain comprising a constant region proximal to the cell membrane and a variable region (i.e., binding domain) that binds to the peptide/MHC. As provided herein, when specified, TCR also includes recombinant TCRs (rTCR or exogenous TCR) having specificity to a tumor associated antigen (TAA) and comprising a transgenic TCRα chain and a transgenic TCRβ chain.

I. Cells and Compositions Useful for Adoptive Cell Therapies with Enhanced Properties

Provided herein is a strategy to systematically engineer the regulatory circuitry of a clonal iPSC without impacting the differentiation potency of the iPSC and cell development biology of the iPSC and its derivative cells, while enhancing the therapeutic properties of the derivative cells differentiated from the iPSC. The iPSC-derived cells are functionally improved and suitable for adoptive cell therapies following a combination of selective modalities being introduced to the cells at the level of iPSC through genomic engineering. It was previously unclear whether altered iPSCs comprising one or more provided genetic edits still have the capacity to enter cell development, and/or to mature and generate functional differentiated cells while retaining modulated activities and/or properties. Unanticipated failures during directed cell differentiation from iPSCs have been attributed to aspects including, but not limited to, development stage specific gene expression or lack thereof, requirements for HLA complex presentation, protein shedding of introduced surface expressing modalities, and the need for reconfiguration of differentiation protocols enabling phenotypic and/or functional changes in the cell. The present application shows that the one or more selected genomic modifications as provided herein do not negatively impact iPSC differentiation potency, and the functional effector cells derived from the engineered iPSC have enhanced and/or acquired therapeutic properties attributable to the individual or combined genomic modifications retained in the effector cells following the iPSC differentiation. Further, all genomic modifications and combinations thereof as may be described in the context of iPSC and iPSC-derived effector cells are applicable to primary sourced cells, including primary immune cells such as T, NK, or immunoregulatory cells, whether cultured or expanded, the modification of which results in engineered immune cells useful for adoptive cell therapy.

1. Exogenous TCR Specific to Tumor Associated Antigen

Alpha-beta T cell receptors (TCRαβ) are antigen specific receptors essential to the immune response and are present on the cell surface of αβ T lymphocytes. Binding of TCRαβ to peptide-major histocompatibility complex (pMHC) initiates TCR-CD3 intracellular activation, recruitment of numerous signaling molecules, and branching and integrating signaling pathways, leading to mobilization of transcription factors that are critical for gene expression and T cell growth and function acquisition. Disrupting the constant region of TCR alpha or TCR beta (TRAC or TRBC), either through direct editing of a T cell or through genomic iPSC editing and differentiation as a source for obtaining a modified derivative T lineage cell, is one of the approaches to produce a TCR^{neg} T cell. As used herein, the term “TCR negative” or “TCR^{neg}” refers to the lack of endogenous TCR expression, either due to TCR gene expression disruption by editing the constant region of any TCR chain or due to a natural absence of TCR gene expression despite the existence of the TCR locus in the genome. TCR^{neg} T cells do not require HLA matching, have reduced alloreactivity, and are able to prevent GvHD (Graft versus Host Disease) when used in allogeneic adoptive cell therapies.

However, in the case of iPSC reprogrammed from T cells (TiPSC), it is discovered here that disrupting only TRAC or only TRBC can lead to formation of a mispaired TCR complex resulting from expression of the remaining endogenous TCR chain (either TRAC or TRBC) by pairing the endogenous TCR chain with an inserted exogenous TCR chain. For example, transduction of a transgenic TCRα chain (tgTCRα) and a transgenic TCRβ chain (tgTCRβ) into cells having TRAC knockout only can result in a mispair between the exogenous TCR alpha chain and the endogenous TCR beta chain in the transduced effector cells, which results in a non-functional mispaired TCR.

TCR disruption also results in the elimination of the CD3 signaling complex from the T cell surface despite the endogenous CD3 subunit gene expression in the cell. The lack of cell surface CD3 may alter the cell's capacity for expansion and/or survival and reduce the cell's functional potential due to incompatibility with technologies requiring cell surface CD3 recognition and binding, which include, but are not limited to, CD3-based antibody and engager technologies; CD3/CD28 T cell activation bead technology; and CD3-CAR stimulation technology. Further, when TCR^{neg} iPSCs are used for directed T cell differentiation, there may also be undesirable impacts on T cell development biology and on T cell function maturation. However, overexpressing CD3 in cells that are TCR negative does not seem to restore cell surface presentation of the CD3 complex and/or CD3 signaling. As for cells that do not express CD3 and/or TCR despite the existence of TCR genes, for example, NK or NK progenitor cells, the acquired CD3 surface expression enables specific signal transduction and cell functions in NK lineage cells via CD3-based antibodies, engagers and CAR technologies that would not have been naturally compatible with these cells.

As provided herein, in various embodiments, both endogenous TCRα and endogenous TCRβ are knocked out (TCRα^{-/-} and TCRβ^{-/-}; or TCRα^{neg} and TCRβ^{neg}) in a cell using a genomic editing tool, leading to a TCR^{neg} cell. In some other embodiments, only endogenous TCRα is knocked out which also results in a TCR^{neg} cell. Simultaneously with, or subsequently to, the TCR knockout, a first polynucleotide encoding a TCRα comprising a defined variable region of TCRα and a full or partial constant region (tgTCRα), and a second polynucleotide encoding a TCRβ comprising a defined variable region of TCRβ and a full or partial constant region (tgTCRβ) are introduced to the TCR^{neg} cell. A defined TCRα or TCRβ variable region can be of any given specificity to an antigen such that its sequence has been, or can be, identified. A defined TCRα or TCRβ variable region can also be specifically selected to target a selected tumor associated antigen (TAA). The tumor associated antigen is presented as peptides in the major histocompatibility complex (MHC) on the cell surface, which interact with the T cell receptors (TCR) on an effector T cell to stimulate an anti-tumor response.

In some alternative embodiments, the tgTCRα chain and the tgTCRβ chain form an exogenous TCR complex (TCR^{exo}) comprising an antigen recognition region that recognizes a tumor associated antigen (TAA). In certain embodiments, said antigen recognition region comprised in the exogenous TCRα and TCRβ is derived from sources comprising a murine antibody, a human antibody, a humanized antibody, a camel Ig, a single variable new antigen receptor (VNAR), a shark heavy-chain-only antibody (Ig NAR), a chimeric antibody, a recombinant antibody, or an antibody fragment thereof. Non-limiting examples of antibody fragments include Fab, Fab′, F(ab′)2, F(ab′)3, Fv, single chain antigen binding fragment (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody.

In some embodiments, one or both of the first and the second polynucleotides encoding TCRα and TCRβ is driven by an endogenous promoter of TCRα and TCRβ, respectively. In some other embodiments, one or both of the first and the second polynucleotides is driven by an exogenous promoter. In some embodiments, the second polynucleotide is driven by an endogenous promoter of TCRβ, whereas in some other embodiments, the second polynucleotide is driven by an exogenous promoter. In some embodiments, the exogenous promoter comprises a constitutive, inducible, temporal-, tissue-, or cell type-specific promoter. In some embodiments, the exogenous promoter comprises one of CMV, EF1α, PGK, CAG, or UBC. In one embodiment, the exogenous promoter comprises at least CAG.

In some embodiments, the polynucleotide encoding a full or partial length of TCRα constant region and a given defined variable region comprises at least a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 1. In some embodiments, the polynucleotide encoding a full or partial length of TCRβ constant region and a given defined variable region comprises at least a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 2 or SEQ ID NO: 3. In some embodiments, the sequence identity is at least 80%. In some embodiments, the sequence identity is at least 90%. In some embodiments, the sequence identity is at least 95%. In some embodiments, the sequence identity is 100%. In some embodiments of the polynucleotide encoding a full length of TCRα or TCRβ constant region, the polynucleotide further comprises a polyA tail at the C′ terminus. In some embodiments of the polynucleotide encoding a partial length of TCRα or TCRβ constant region, the integration of the polynucleotide is at a site within the endogenous constant region and is in-frame with the remaining endogenous sequence of TCRα or TCRβ constant region downstream of the integration site, such that a full length transgenic/chimeric TRAC or TRBC is formed with a part of its sequence being exogenous/transgenic and another part being endogenous. Exemplary N-terminal signal peptides include MALPVTALLLPLALLLHA (SEQ ID NO: 4; CD8asp) or MDFQVQIFSFLLISASVIMSR (SEQ ID NO: 5; IgKsp), or any signal peptide sequence or functional variants thereof known in the art. An exemplary linker peptide includes DYKDDDDK (SEQ ID NO: 6; FLAG), or any linker peptide sequence or functional variants thereof known in the art.

SEQ ID NO: 1:
IQNPDPAVYQLRDSKSSDKSVCLFTDFDSQTNVSQSKDSD
VYITDKTVLDMRSMDFKSNSAVAWSNKSDFACANAFNNSI
IPEDTFFPSPESSCDVKLVEKSFETDTNLNFQNLSVIGFR
ILLLKVAGENLLMTLRLWSS
(TRAC)
SEQ ID NO: 2:
DLNKVFPPEVAVFEPSEAEISHTQKATLVCLATGFFPDHV
ELSWWVNGKEVHSGVSTDPQPLKEQPALNDSRYCLSSRLR
NHERCRVSATFWQNPQVQFYGLSENDEWTQDRAKPVTQIV
SAEAWGRADCGFTSVSYQQGVLSATILYEILLGKATLYAV
LVSALVLMAMVKRKDE
(TRBC1)
SEQ ID NO: 3
DLKNVFPPKVAVFEPSEAEISHTQKATLVCLATGFYPDHV
ELSWWVNGKEVHSGVSTDPQPLKEQPALNDSRYCLSSRLR
VSATFWQNPRNHFRCQVQFYGLSENDEWTQDRAKPVTQIV
SAEAWGRADCGFTSESYQQGVLSATILYEILLGKATLYAV
LVSALVLMAMVKRKDSRG
(TRBC2)

As discussed herein, a transgenic TCRα (tgTCRα) having a constant region and a defined variable region, optionally with a transgenic TCRβ (tgTCRβ) having a constant region and a defined variable region is capable of forming an exogenous TCR complex (TCR^{exo}) by associating with the endogenous CD3 subunits including the CD3ζ chain. Such a TCR^{exo} complex has a defined (or selected, or targeted), or no, peptide-WIC binding due to the specificity of the selected variable region of tgTCRα and tgTCRβ. In some embodiments, the tgTCRα chain and the tgTCRβ chain are inserted at a constant region of TCRα or TCRβ (TRAC or TRBC), thereby disrupting expression of endogenous TCRα or TCRβ at the insertion site. In some other embodiments, an exogenous variable portion of a TCR is inserted at the TCR locus, such that a recombinant TCR is expressed, where the recombinant TCR comprises the exogenous variable portion operatively linked to the endogenous constant region of the TCR.

In this application, MR1-TCR is provided for proof-of-concept purposes in providing engineered immune effector cells with enhanced functionality. Major histocompatibility complex class related protein 1 (MR1) is a non-classical WIC class I protein that is widely expressed with minimal variability among patients and enables the unique prospect to be a universal cancer immunotherapy. As such, in one example, the TCR^{exo} formed in the cell is specific for MR1. In embodiments where the TCR^{exo} is specific for MR1, the tgTCRα comprises a variable alpha (Vα) fragment that comprises a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 7 (MR1Vα), and a TCRα constant fragment comprising a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 8. In some embodiments of the TCR^{exo} that is specific for MR1, the tgTCRβ comprises a variable beta (Vβ) fragment that comprises a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 9, and a TCRβ constant fragment comprising a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 10. Another aspect of the present specification provides a genetically engineered iPSC, its derivative cell, or a population thereof, wherein the cell comprises an exogenous polynucleotide encoding at least the tgTCRα comprising a variable alpha (Vα) fragment and a TCRα constant fragment; and/or a variable beta (Vβ) fragment and a TCRβ constant fragment. In some embodiments, the integration of the exogenous TCR chain polynucleotide is at a site within an endogenous constant region of the cell and thereby disrupts the expression of the endogenous TCR chain and the endogenous TCR complex.

SEQ ID NO: 7
MAQTVTQSQPEMSVQEAETVTLSCTYDTSESDYYLFWYKQ
PPSROMILVIRQEAYKQQNATENRESVNFQKAAKSFSLKI
SDSQLGDAAMYFCAYRSAVNARLMFGDGTQLVVKPN
(underlined is a variable region)
SEQ ID NO: 8
IQNDPAVYQLRDKSSDKSVCLFTDFDSQTNVSQSKDSDVY
ITDKCVLDMRSMDFKSNSAVAWSNKSDFACANAFNNSIIP
EDTFFPSPESSCDVKLVEKSFETDTNLNFQNLSVIGFRIL
LLKVAGENLLMTLRLWSS
SEQ ID NO: 9
MEADIYQTPRYLVIGTGKKITLECSQTMGHDKMYWYQQDP
GMELHLIHYSYGVNSTEKGDLSSESTVSRIRTEHFPLTLE
SARPSHTSQYLCASSEARGLAEFTDTQYFGPGTRLTVLE
(underlined is a variable region)
SEQ ID NO: 10
LKNVFPPEVVFEPSEAEISHTQKATLVCLATGFYPDHVEL
SWWVNGKEVHSGVCTDPQPLKEQPALNDSRYALSSRLRVS
ATFWQDPRNHFRCQVQFYGLSENDEWTQDRAKPVTQIVSA
EAWGRADCGFTSESYQQGVLSATILYEILLGKATLYAVLV
SALVLMAMVKRKDSRG
(underlined is a variable region)

As another exemplary antigen specific TCR, MICA/B recognition is incorporated to generate a recombinant/exogenous TCR. MICA and MICB are expressed family members of the human major histocompatibility complex class I chain-related gene (MIC). The members of the MIC family are highly polymorphic (more than 100 human alleles), but with structurally conserved motifs. MICA/B as a tumor associated antigen is predominantly expressed in GI epithelium, endothelial cells and fibroblasts, with its expression being induced by cellular/genotoxic stress, and it has high expression on epithelial and melanoma cancers. The shedding of MICA/B on tumor cells, on the other hand, results in increased soluble MICA/B which is not recognized by NKG2D expressed on NK and T cell subsets, and possibly enables tumor evasion/escape and inhibits immunosurveillance. Thus, in another example, the TCR^{exo} formed in the cell targets tumor antigen MICA and MICB. In some embodiments, the antigen recognition region is a scFV that specifically binds to the conserved α3 domain of MICA and MICB. In one embodiment, the scFV comprises a variable region of the heavy chain represented by an amino acid sequence that is of at least about 99%, about 98%, about 96%, about 95%, about 90%, about 85%, or at least about 80% identity to SEQ ID NO: 11, and a variable region of the light chain represented by an amino acid sequence that is of at least about 99%, about 98%, about 96%, about 95%, about 90%, about 85%, or at least about 80% identity to SEQ ID NO: 12. In one embodiment of the MICA/B scFV, the scFV is represented by an amino acid sequence that is of at least about 99%, about 98%, about 96%, about 95%, about 90%, about 85%, or at least about 80% identity to SEQ ID NO: 13, in which the linker and/or signal peptide are exemplary and are replaceable. In another embodiment of the MICA/B scFV, the scFV is represented by an amino acid sequence that is of at least about 99%, about 98%, about 96%, about 95%, about 90%, about 85%, or at least about 80% identity to SEQ ID NO: 14, in which the linker and/or signal peptide are exemplary and their length and sequence can vary. Another aspect of the present specification provides a genetically engineered iPSC and its derivative cell, wherein the cell comprises an exogenous polynucleotide encoding at least a MICA/B scFV.

SEQ ID NO: 11
QIQLVQSGPELKKPGETVKVSCKASGYMFTNYAMNWVKQA
PEKGLKWMGWINTHTGDPTYADDEKGRIAFSLETSASTAY
LQINNLKNEDTATYFCVRTYGNYAMDYWGQGTSVTVSS
(118 AA. MICA/B scFV heavy chain (HC))
SEQ ID NO: 12
DIQMTQTTSSLSASLGDRVTISCSASQDISNYLNWYQQKP
DGTVKLLIYDTSILHLGVPSRESGSGSGTDYSLTISNLEP
EDIATYYCQQYSKFPRTFGGGTTLEIK
(107 AA. MICA/B scFV light chain (LC))
(HC-Linker-LC)
SEQ ID NO: 13
MDFQVQIFSELLISASVIMSRQIQLVQSGPELKKPGETVK
VSCKASGYMFTNYAMNWVKQAPEKGLKWMGWINTHTGDPT
YADDEKGRIAFSLETSASTAYLQINNLKNEDTATYFCVRT
YGNYAMDYWGQGTSVTVSSGGGGSGGGGSGGGGSDIQMTQ
TTSSLSASLGDRVTISCSASQDISNYLNWYQQKPDGTVKL
LIYDTSILHLGVPSRFSGSGSGTDYSLTISNLEPEDIATY
YCQQYSKFPRTFGGGTTLEIK
(Signal peptide-other signal peptides
are also possible;
Linker-other linkers are also possible)
(LC-Linker-HC)
SEQ ID NO: 14
MDFQVQIFSFLLISASVIMSRDIQMTQTTSSLSASLGDRV
TISCSASQDISNYLNWYQQKPDGTVKLLIYDTSILHLGVP
SRFSGSGSGTDYSLTISNLEPEDIATYYCQQYSKFPRTFG
GGTTLEIKGGGGSGGGGSGGGGSQIQLVQSGPELKKPGET
VKVSCKASGYMFTNYAMNWVKQAPEKGLKWMGWINTHTGD
PTYADDFKGRIAFSLETSASTAYLQINNLKNEDTATYFCV
RTYGNYAMDYWGQGTSVTVSS
(Signal peptide-other signal peptides
are also possible;
Linker-other linkers are also possible)

As provided further, the cell, or a population thereof comprising a polynucleotide encoding a tgTCRα and a tgTCRβ may further comprise one or more additional engineered modalities described herein. Further provided herein is a master cell bank comprising single cell sorted and expanded clonal engineered iPSCs having at least one phenotype listed in Table 1, wherein the cell bank provides a platform for additional iPSC engineering and a renewable source for manufacturing off-the-shelf, engineered, homogeneous cell therapy products.

2. Chimeric Antigen Receptor (CAR) Expression

Applicable to the genetically engineered iPSC and derivative effector cell thereof may be any CAR design known in the art. CAR is a fusion protein generally including an ectodomain that comprises an antigen recognition region, a transmembrane domain, and an endodomain. In some embodiments, the ectodomain can further include a signal peptide or leader sequence and/or a spacer. In some embodiments, the endodomain can further comprise a signaling peptide that activates the effector cell expressing the CAR. In some embodiments, the endodomain can further comprise a signaling domain, where the signaling domain originates from a cytoplasmic domain of a signal transducing protein specific to T and/or NK cell activation or functioning. In some embodiments, the antigen recognition domain can specifically bind an antigen. In some embodiments, the antigen recognition domain can specifically bind an antigen associated with a disease or pathogen. In some embodiments, the disease-associated antigen is a tumor antigen, wherein the tumor may be a liquid or a solid tumor. In some embodiments, the CAR is suitable to activate either T or NK lineage cells expressing the CAR. In some embodiments, the CAR is NK cell specific for comprising NK-specific signaling components. In certain embodiments, the T cells are derived from a CAR-expressing iPSC, and the derivative T lineage cells may comprise T helper cells, cytotoxic T cells, memory T cells, regulatory T cells, natural killer T cells, αβ T cells, γδ T cells, or a combination thereof. In certain embodiments, the NK cells are derived from CAR-expressing iPSCs.

In certain embodiments, said antigen recognition region/domain comprises a murine antibody, a human antibody, a humanized antibody, a camel Ig, a single variable new antigen receptor (VNAR), a shark heavy-chain-only antibody (Ig NAR), a chimeric antibody, a recombinant antibody, or an antibody fragment thereof. Non-limiting examples of antibody fragments include Fab, Fab′, F(ab′)2, F(ab′)3, Fv, single chain antigen binding fragment (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody. In some embodiments, the antigen recognition region of a CAR originates from the binding domain of a T cell receptor (TCR) that targets a tumor associated antigen (TAA).

Non-limiting examples of antigens that may be targeted by a CAR include ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), K-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NYESO1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and various pathogen antigens known in the art. Non-limiting examples of pathogens include viruses, bacteria, fungi, parasites and protozoa capable of causing diseases. In still some other embodiments, the CAR-T cell comprising an exogenous TCR complex further comprises a CAR specific to one of MR1, NYESO1, MICA/B, EpCAM, EGFR, B7H3, Muc1, Muc16, CD19, BCMA, CD20, CD22, CD38, CD123, HER2, CD52, GD2, MSLN, VEGF-R2, PSMA and PDL1.

Some aspects of the invention provide a CAR binding domain that originates from a T cell receptor for a tumor associated antigen. In various embodiments, the CAR is specific to a tumor cell surface antigen presented by the MR1 protein (MR1-CAR). In some embodiments, the antigen recognition domain of the ectodomain of the MR1-CAR comprises a variable alpha (Vα) fragment and a variable beta (Vβ) fragment. In some embodiments, the variable alpha (Vα) fragment comprises at least a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 7 (MR1Vα). In some embodiments, the variable beta (Vβ) fragment comprises at least a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 9 (MR1Vβ).

In some embodiments, the antigen recognition domain of the ectodomain of the MR1-CAR comprises an extracellular domain of MR1 TCRα and an extracellular domain of MR1 TCRβ. In some embodiments, the extracellular domain of MR1 TCRα comprises at least a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 15. In some embodiments, the extracellular domain of MR1 TCRβ comprises at least a sequence having an identity of at least 50%, 55%, 60%, 65%, 70%, 75%, 80%, 85%, 90%, 95%, 99%, 100%, or any percentage in-between, when compared to the exemplary sequence, SEQ ID NO: 16.

SEQ ID NO: 15
MAQTVTQSQPEMSVQEAETVTLSCTYDTSESDYYLFWYKQ
PPSRQMILVIRQEAYKQQNATENRFSVNFQKAAKSFSLKI
SDSQLGDAAMYFCAYRSAVNARLMFGDGTQLVVKPNIQNP
DPAVYQLRDSKSSDKSVCLFTDFDSQTNVSQSKDSDVYIT
DKCVLDMRSMDFKSNSAVAWSNKSDFACANAFNNSIIPED
TFFPSPESSCDVKLVEKSFETDINLNFQNLS
SEQ ID NO: 16
MEADIYQTPRYLVIGTGKKITLECSQTMGHDKMYWYQQDP
GMELHLIHYSYGVNSTEKGDLSSESTVSRIRTEHFPLTLE
SARPSHTSQYLCASSEARGLAEFTDTQYFGPGTRLTVLED
LKNVFPPEVAVFEPSEAEISHTQKATLVCLATGFYPDHVE
LSWWVNGKEVHSGVCTDPQPLKEQPALNDSRYCLSSRLRV
SATFWQNPRNHFRCQVQFYGLSENDEWTQDRAKPVTQIVS
AEAWGRADCGFTSESYQQGVLSA
(underlined is a variable region)

Another aspect of the present specification provides a genetically engineered iPSC and its derivative cell, wherein the cell comprises an exogenous polynucleotide encoding at least a MR1-CAR. In some embodiments, the iPSC-derived effector cell comprising an exogenous polynucleotide encoding at least a MR1-CAR is a T cell. In some embodiments, the iPSC-derived effector cell comprising an exogenous polynucleotide encoding at least a MR1-CAR is an NK cell. In some other embodiments, the iPSC-derived effector cell comprising an exogenous polynucleotide encoding at least a MR1-CAR is an NKT cell.

In another example, the present specification provides a CAR comprising an antigen recognition region that targets the tumor antigen MICA and MICB. In some embodiments of the MICA/B targeting CAR, the antigen recognition region is a scFV that specifically binds to the conserved α3 domain of MICA and MICB. In one embodiment, the scFV comprises a variable region of the heavy chain represented by an amino acid sequence that is of at least about 99%, about 98%, about 96%, about 95%, about 90%, about 85%, or at least about 80% identity to SEQ ID NO: 11, and a variable region of the light chain represented by an amino acid sequence that is of at least about 99%, about 98%, about 96%, about 95%, about 90%, about 85%, or at least about 80% identity to SEQ ID NO: 12. In one embodiment of the MICA/B scFV, the scFV is represented by an amino acid sequence that is of at least about 99%, about 98%, about 96%, about 95%, about 90%, about 85%, or at least about 80% identity to SEQ ID NO: 13, in which the linker and/or signal peptides are exemplary and are replaceable. In another embodiment of the MICA/B scFV, the scFV is represented by an amino acid sequence that is of at least about 99%, about 98%, about 96%, about 95%, about 90%, about 85%, or at least about 80% identity to SEQ ID NO: 14, in which the linker and/or signal peptides are exemplary and their length and sequence can vary. Another aspect of the present specification provides a genetically engineered iPSC and its derivative cell, wherein the cell comprises an exogenous polynucleotide encoding at least a MICAS-CAR. In some embodiments, the iPSC-derived effector cell comprising an exogenous polynucleotide encoding at least a MICAS-CAR is a T cell. In some embodiments, the iPSC-derived effector cell comprising an exogenous polynucleotide encoding at least a MICAS-CAR is an NK cell. In some other embodiments, the iPSC-derived effector cell comprising an exogenous polynucleotide encoding at least a MICA/B-CAR is an NKT cell.

In some embodiments, the transmembrane domain of a CAR comprises a full length or at least a portion of the native or modified transmembrane region of CD2, CD3δ, CD3ε, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide.

In some embodiments, the signal transducing peptide of the endodomain (or intracellular domain) comprises a full length or at least a portion of a polypeptide of 2B4 (Natural killer Cell Receptor 2B4), 4-1BB (Tumor necrosis factor receptor superfamily member 9), CD16 (IgG Fc region Receptor III-A), CD2 (T cell surface antigen CD2), CD28 (T cell-specific surface glycoprotein CD28), CD28H (Transmembrane and immunoglobulin domain-containing protein 2), CD3ζ (T cell surface glycoprotein CD3 zeta chain), CD3ζ1XX (CD3ζ variant), DAP10 (Hematopoietic cell signal transducer), DAP12 (TYRO protein tyrosine kinase-binding protein), DNAM1 (CD226 antigen), FcERIγ (High affinity immunoglobulin epsilon receptor subunit gamma), IL21R (Interleukin-21 receptor), IL-2Rβ/IL-15RB (Interleukin-2 receptor subunit beta), IL-2Rγ (Cytokine receptor common subunit gamma), IL-7R (Interleukin-7 receptor subunit alpha), KIR2DS2 (Killer cell immunoglobulin-like receptor 2DS2), NKG2D (NKG2-D type II integral membrane protein), NKp30 (Natural cytotoxicity triggering receptor 3), NKp44 (Natural cytotoxicity triggering receptor 2), NKp46 (Natural cytotoxicity triggering receptor 1), CS1 (SLAM family member 7), and CD8 (T cell surface glycoprotein CD8 alpha chain).

In some embodiments, the endodomain of a CAR further comprises a second signaling domain, and optionally a third signaling domain, where each of the first, second, and third signaling domains are different. In particular embodiments, the second or the third signaling domain comprises a cytoplasmic domain, or a portion thereof, of 2B4, 4-1BB, CD16, CD2, CD28, CD28H, CD3ζ, DAP10, DAP12, DNAM1, FcERIγ IL21R, (IL-15Rβ), IL-2Rγ, IL-7R, KIR2DS2, NKG2D, NKp30, NKp44, NKp46, CD3 ζ1XX, CS1, or CD8.

In certain embodiments, the endodomain further comprises at least one co-stimulatory signaling region. Said co-stimulatory signaling region can comprise a full length or at least a portion of a polypeptide of CD27, CD28, 4-1BB, OX40, ICOS, PD-1, LAG-3, 2B4, BTLA, DAP10, DAP12, CTLA-4, or NKG2D, or any combination thereof.

In one embodiment, the CAR applicable to the cells provided herein comprises a co-stimulatory domain derived from CD28, and a signaling domain comprising the native or modified ITAM1 of CD3ζ, represented by an amino acid sequence having at least about 85%, about 90%, about 95%, about 96%, about 97%, about 98%, or about 99% identity to SEQ ID NO: 17. In a further embodiment, the CAR comprising a co-stimulatory domain derived from CD28, and a native or modified ITAM1 of CD3ζ also comprises a hinge domain and transmembrane domain derived from CD28, wherein an scFv may be connected to the transmembrane domain through the hinge, and the CAR comprises an amino acid sequence of at least about 85%, about 90%, about 95%, about 96%, about 97%, about 98%, or about 99% identity to SEQ ID NO: 18. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 17
RSKRSRLLHSDYMNMTPRRPGPTRKHYQPYAPPRDFAAYR
SRVKFSRSADAPAYQQGQNQLYNELNLGRREEYDVLDKRR
GRDPEMGGKPRRKNPQEGLFNELQKDKMAEAFSEIGMKGE
RRRGKGHDGLFQGLSTATKDTFDALHMQALPPR
(153 a.a. CD28 co-stim + CD3ζITAM)
SEQ ID NO: 18
IEVMYPPPYLDNEKSNGTIIHVKGKHLCPSPLFPGPSKPF
WVLVVVGGVLACYSLLVTVAFIIFWVRSKRSRLLHSDYMN
MTPRRPGPTRKHYQPYAPPRDFAAYRSRVKFSRSADAPAY
QQGQNQLYNELNLGRREEYDVLDKRRGRDPEMGGKPRRKN
PQEGLFNELQKDKMAEAFSEIGMKGERRRGKGHDGLFQGL
STATKDTFDALHMQALPPR
(219 a.a. CD28 hinge + CD28 TM +
CD28 co-stim + CD3ζITAM)

In another embodiment, the CAR applicable to the cells provided herein comprises a transmembrane domain derived from NKG2D, a co-stimulatory domain derived from 2B4, and a signaling domain comprising the native or modified CD3ζ, represented by an amino acid sequence of at least about 85%, about 90%, about 95%, about 96%, about 97%, about 98%, or about 99% identity to SEQ ID NO: 19. Said CAR comprising a transmembrane domain derived from NKG2D, a co-stimulatory domain derived from 2B4, and a signaling domain comprising the native or modified CD3ζ may further comprise a CD8 hinge, wherein the amino acid sequence of such a structure is of at least about 85%, about 90%, about 95%, about 96%, about 97%, about 98%, or about 99% identity to SEQ ID NO: 20. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 19
SNLFVASWIAVMIIFRIGMAVAIFCCFFFPSWRRKRKEKQ
SETSPKEFLTIYEDVKDLKTRRNHEQEQTFPGGGSTIYSM
IQSQSSAPTSQEPAYTLYSLIQPSRKSGSRKRNHSPSENS
TIYEVIGKSQPKAQNPARLSRKELENEDVYSRVKESRSAD
APAYKQGQNQLYNELNLGRREEYDVLDKRRGRDPEMGGKP
RRKNPQEGLYNELQKDKMAEAYSEIGMKGERRRGKGHDGL
YQGLSTATKDTYDALHMQALPPR
(263 a.a NKG2D TM + 2B4 + CD32)
SEQ ID NO: 20
TTTPAPRPPTPAPTIASQPLSLRPEACRPAAGGAVHTRGL
DFACDSNLFVASWIAVMIIFRIGMAVAIFCCFFFPSWRRK
RKEKQSETSPKEFLTIYEDVKDLKTRRNHEQEQTFPGGGS
TIYSMIQSQSSAPTSQEPAYTLYSLIQPSRKSGSRKRNHS
PSENSTIYEVIGKSQPKAQNPARLSRKELENFDVYSRVKE
SRSADAPAYKQGQNQLYNELNLGRREEYDVLDKRRGRDPE
MGGKPRRKNPQEGLYNELQKDKMAEAYSEIGMKGERRRGK
GHDGLYQGLSTATKDTYDALHMQALPPR
(308 a.a CD8 hinge + NKG2D TM + 2B4 + CD3)

Non-limiting CAR strategies further include heterodimeric, conditionally activated CAR through dimerization of a pair of intracellular domains (see for example, U.S. Pat. No. 9,587,020); split CAR, where homologous recombination of antigen binding, hinge, and endodomains to generate a CAR (see for example, U.S. Pub. No. 2017/0183407); multi-chain CAR that allows non-covalent link between two transmembrane domains connected to an antigen binding domain and a signaling domain, respectively (see for example, U.S. Pub. No. 2014/0134142); CARs having bi-specific antigen binding domains (see for example, U.S. Pat. No. 9,447,194), or having a pair of antigen binding domains recognizing the same or different antigens or epitopes (see for example, U.S. Pat. No. 8,409,577), or a tandem CAR (see for example, Hegde et al., J Clin Invest. 2016; 126(8):3036-3052); inducible CAR (see for example, U.S. Pub. Nos. 2016/0046700, 2016/0058857, and 2017/0166877); switchable CAR (see for example, U.S. Pub. No. 2014/0219975); and any other designs known in the art.

In a further embodiment, the iPSC and its derivative effector cells comprising a CAR and TCR^{exo} have the CAR inserted in a TCR constant region, leading to endogenous TCR knockout, and placing CAR expression under the control of the endogenous TCR promoter. In some other embodiments, the CAR inserted in the TCR constant region is specific to a tumor antigen comprising at least one of MR1, NYESO1, MICA/B, EpCAM, EGFR, B7H3, Muc1, Muc16, CD19, BCMA, CD20, CD22, CD38, CD123, HER2, CD52, GD2, MSLN, VEGF-R2, PSMA and PDL1. Additional CAR insertion sites include, but are not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, NKG2A, NKG2D, CD25, CD38, CD44, CD58, CD54, CD56, CD69, CD71, CIS, CBL-B, SOCS2, PD1, CTLA4, LAG3, TIM3, and TIGIT. In some embodiments, an iPSC or its derivative T cell comprising TCR^{exo} and a CAR, and optionally a CFR or engager further comprises an exogenous CD16 having an ectodomain native to CD16 (F176V and/or S197P) or derived from CD64, and native or non-native transmembrane, stimulatory and signaling domains. In another embodiment, the iPSC and its derivative NK cells comprise TCR^{exo} and a CAR, where the CAR is inserted in the NKG2A locus or NKG2D locus, leading to NKG2A or NKG2D knockout, thereby placing CAR expression under the control of the endogenous NKG2A or NKG2D promoter.

As such, in addition to genetically engineered immune cells comprising the functional modalities as provided herein, aspects of the present invention provide derivative cells obtained from differentiating genomically engineered iPSCs, wherein both the iPSCs and the derivative cells comprise one or more CARs along with additional modified modalities, as discussed herein. Additionally provided in this application is a master cell bank comprising single cell sorted and expanded clonal engineered iPSCs having at least a CAR and TCR^{exo}, wherein the cell bank provides a platform for additional iPSC engineering and a renewable source for manufacturing off-the-shelf, engineered, homogeneous cell therapy products.

3. CD16 Knock-In

CD16 has been identified as two isoforms, Fc receptors FcγRIIIa (CD16a; NM 000569.6) and FcγRIIIb (CD16b; NM 000570.4). CD16a is a transmembrane protein expressed by NK cells, which binds monomeric IgG attached to target cells to activate NK cells and facilitate antibody-dependent cell-mediated cytotoxicity (ADCC). CD16b is exclusively expressed by human neutrophils. “High affinity CD16,” “non-cleavable CD16,” or “high affinity non-cleavable CD16” (abbreviated as hnCD16), as used herein, refers to various CD16 variants. The wildtype CD16 has low affinity and is subject to down regulation including ectodomain shedding, a proteolytic cleavage process that regulates the cells surface density of various cell surface molecules on leukocytes upon NK cell activation. F176V (also called F158V in some publications) is an exemplary CD16 polymorphic allele/variant having high affinity; whereas S197P variant is an example of a genetically engineered non-cleavable version of CD16. An engineered CD16 variant comprising both F176V and S197P has high affinity and is non-cleavable, which was described in greater detail in WO2015/148926, the complete disclosure of which is incorporated herein by reference. In addition, a chimeric CD16 receptor with the ectodomain of CD16 essentially replaced with at least a portion of CD64 ectodomain can also achieve the desired high affinity and non-cleavable features of a CD16 receptor capable of carrying out ADCC. In some embodiments, the replacement ectodomain of a chimeric CD16 comprises one or more of EC1, EC2, and EC3 exons of CD64 (UniPRotKB_P12314 or its isoform or polymorphic variant).

As such, various embodiments of an exogenous CD16 introduced to a cell include functional CD16 variants and chimeric receptors thereof. In some embodiments, the functional CD16 variant is a high-affinity non-cleavable CD16 receptor (hnCD16). An hnCD16, in some embodiments, comprises both F176V and S197P; and in some embodiments, comprises F176V and with the cleavage region eliminated. In some other embodiments, a hnCD16 comprises at least a portion of the CD64 ectodomain.

Accordingly, provided herein are clonal iPSCs genetically engineered to comprise, among other editing as contemplated and described herein, an exogenous CD16 or variant thereof introduced to the iPSCs. In some embodiments, the exogenous CD16 is a high-affinity non-cleavable CD16 receptor (hnCD16). In some embodiments, the exogenous CD16 comprises at least a portion of the CD64 ectodomain. In some embodiments the exogenous CD16 is in a form of CD16-based chimeric Fc receptor (CFcR) that comprises a transmembrane domain, a stimulatory domain and/or a signaling domain that is not derived from CD16.

In some embodiments, the primary-sourced or derived effector cells comprising the exogenous CD16 or a variant thereof are NK lineage cells. In some embodiments, the primary-sourced or derived effector cells comprising the exogenous CD16 or a variant thereof are T lineage cells. In some embodiments, the exogenous CD16 comprises hnCD16. In some embodiments, the hnCD16 comprises a full or a partial length extracellular domain of CD64. The exogenous CD16 or functional variants thereof comprised in iPSC or derivative cells thereof has high affinity in binding to a ligand that triggers downstreaming signaling upon the binding. The ligand binding to the exogenous CD16 or functional variants include not only ADCC antibodies or fragments thereof, but also bi-, tri-, or multi-specific engagers or binders that recognize the CD16 or CD64 extracellular binding domains of said exogenous CD16. As such, at least one of the aspects of the present application provides a derivative effector cell or a cell population thereof preloaded with one or more pre-selected ADCC antibodies through an exogenous CD16 expressed on the effector cell, in an amount sufficient for therapeutic use in a treatment of a condition, a disease, or an infection as further detailed in this application, wherein said exogenous CD16 comprises an extracellular binding domain of CD64, or of CD16 having F176V and S197P.

In some other embodiments, an exogenous CD16 comprises a CD16-, or variants thereof, based CFcR. A chimeric Fc receptor (CFcR) is produced to comprise a non-native transmembrane domain, a non-native stimulatory domain and/or a non-native signaling domain by modifying or replacing the native CD16 transmembrane- and/or the intracellular-domain. The term “non-native” used herein means that the transmembrane, stimulatory or signaling domain are derived from a different receptor other than the receptor which provides the extracellular domain. In the illustration here, the CFcR based on CD16 or variants thereof does not have a transmembrane, stimulatory or signaling domain that is derived from CD16. In some embodiments, the exogenous CD16-based CFcR comprises a non-native transmembrane domain derived from CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or a T cell receptor polypeptide. In some embodiments, the exogenous CD16-based CFcR comprises a non-native stimulatory/inhibitory domain derived from CD27, CD28, 4-1BB, OX40, ICOS, PD-1, LAG-3, 2B4, BTLA, DAP10, DAP12, CTLA-4, or NKG2D polypeptide. In some embodiments, the exogenous CD16-based CFcR comprises a non-native signaling domain derived from CD3ζ, 2B4, DAP10, DAP12, DNAM1, CD137 (4-1BB), IL21, IL7, IL12, IL15, NKp30, NKp44, NKp46, NKG2C, or NKG2D polypeptide. In one embodiment of the CD16-based CFcR, the provided chimeric Fc receptor comprises a transmembrane domain and a signaling domain both derived from one of IL7, IL12, IL15, NKp30, NKp44, NKp46, NKG2C, or NKG2D polypeptide. One particular exemplary embodiment of the CD16-based chimeric Fc receptor comprises a transmembrane domain of NKG2D, a stimulatory domain of 2B4, and a signaling domain of CD3; wherein the extracellular domain of the CFcR is derived from a full length or partial sequence of the extracellular domain of CD64 or CD16, and wherein the extracellular domain of CD16 comprises F176V and S197P. Another exemplary embodiment of the CD16-based chimeric Fc receptor comprises a transmembrane domain and a signaling domain of CD3; wherein the extracellular domain of the CFcR is derived from a full length or partial sequence of the extracellular domain of CD64 or CD16, and wherein the extracellular domain of CD16 comprises F176V and S197P.

The various embodiments of CD16-based chimeric Fc receptor as described above are capable of binding, with high affinity, to the Fc region of an antibody or fragment thereof; or to a bi-, tri-, or multi-specific engager or binder. Upon binding, the stimulatory and/or signaling domains of the chimeric receptor enable the activation and cytokine secretion of the effector cells, and the killing of the tumor cells targeted by the antibody, or said bi-, tri-, or multi-specific engager or binder having a tumor antigen binding component as well as the Fc region. Without being limited by theory, through the non-native transmembrane, stimulatory and/or signaling domains, or through an engager binding to the ectodomain, of the CD16-based chimeric Fc receptor, the CFcR could contribute to effector cells' killing ability while increasing the effector cells' proliferation and/or expansion potential. The antibody and the engager can bring tumor cells expressing the antigen and the effector cells expressing the CFcR into a close proximity, which also contributes to the enhanced killing of the tumor cells. Exemplary tumor antigens for bi-, tri-, multi-specific engagers or binders include, but are not limited to, B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, and ROR1. Some non-limiting exemplary bi-, tri-, multi-specific engagers or binders suitable for engaging effector cells expressing the CD16-based CFcR in attacking tumor cells include CD16 (or CD64)-CD30, CD16 (or CD64)-BCMA, CD16 (or CD64)-IL15-EPCAM, and CD16 (or CD64)-IL15-CD33.

Unlike the endogenous CD16 expressed by primary NK cells which gets cleaved from the cellular surface following NK cell activation, the various non-cleavable versions of CD16 in derivative NK cells avoid CD16 shedding and maintain constant expression. In derivative NK cells, non-cleavable CD16 increases expression of TNFα and CD107a, indicative of improved cell functionality. Non-cleavable CD16 also enhances the antibody-dependent cell-mediated cytotoxicity (ADCC), and the engagement of bi-, tri-, or multi-specific engagers. ADCC is a mechanism of NK cell mediated lysis through the binding of CD16 to antibody-coated target cells. The additional high affinity characteristics of the introduced hnCD16 in a derived NK cell also enables in vitro loading of an ADCC antibody to the NK cell through hnCD16 before administering the cell to a subject in need of a cell therapy. As provided herein, the hnCD16 may comprise F176V and S197P in some embodiments, or may comprise a full or partial length ectodomain originated from CD64, or may further comprise at least one of non-native transmembrane domain, stimulatory domain and signaling domain. As disclosed, the present application also provides a derivative NK cell or a cell population thereof, preloaded with one or more pre-selected ADCC antibodies in an amount sufficient for therapeutic use in a treatment of a condition, a disease, or an infection as further detailed in this application.

Unlike primary NK cells, mature T cells from a primary source (i.e., natural/native sources such as peripheral blood, umbilical cord blood, or other donor tissues) do not express CD16. It was unexpected that an iPSC comprising an expressed exogenous non-cleavable CD16 did not impair the T cell developmental biology and was able to differentiate into functional derivative T lineage cells that not only express the exogenous CD16, but also are capable of carrying out function through an acquired ADCC mechanism. This acquired ADCC in the derivative T lineage cell can additionally be used as an approach for dual targeting and/or to rescue antigen escape often occurred with CAR-T cell therapy, where the tumor relapses with reduced or lost CAR-T targeted antigen expression or expression of a mutated antigen to avoid recognition by the CAR. When said derivative T lineage cell comprises acquired ADCC through exogenous CD16, including functional variants and CD16-based CFcR, expression, and when an antibody targets a different tumor antigen from the one targeted by the CAR, the antibody can be used to rescue CAR-T antigen escape and reduce or prevent relapse or recurrence of the targeted tumor often seen in CAR-T treatment. Such a strategy to reduce and/or prevent antigen escape while achieving dual targeting is equally applicable to NK cells expressing one or more CARs.

As such, embodiments of the present invention provide genetically engineered immune cells and derivative cells, including a derivative T lineage cell, comprising an exogenous CD16 or variant thereof in addition to TCR^{exo} and optionally a CAR, as provided herein. In some embodiments, the exogenous CD16 comprised in the derivative T lineage cell is an hnCD16 that comprises the CD16 ectodomain comprising F176V and S197P. In some other embodiments, the hnCD16 comprised in the derivative T lineage cell comprises a full or partial length ectodomain originated from CD64; or may further comprise at least one of non-native transmembrane domain, stimulatory domain and signaling domain. As explained herein, such derivative T lineage cells have an acquired mechanism to target tumors with a monoclonal antibody meditated by ADCC to enhance the therapeutic effect of the antibody. As disclosed, the present application also provides genetically engineered immune cells and derivative cells, including a derivative T lineage cell, or a population thereof, preloaded with one or more pre-selected ADCC antibodies in an amount sufficient for therapeutic use in a treatment of a condition, a disease, or an infection as further detailed below.

Additionally provided in this application is a master cell bank comprising single cell sorted and expanded clonal engineered iPSCs having at least one phenotype as provided herein, including but not limited to, TCR^{exo}, a CAR, and an exogenous CD16 or a variant thereof, wherein the cell bank provides a platform for additional iPSC engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

4. Engagers

In some embodiments, the iPSC, and its derivative effector cells comprising TCR^{exo} and optionally a CAR and/or exogenous CD16 or a variant thereof, may additionally comprise introduced expression of an engager having a different tumor targeting specificity from the TCR^{exo} and/or CAR (i.e., a second tumor antigen). Engagers are fusion proteins comprising two or more single-chain variable fragments (scFvs), or other functional variants, of different antibodies or fragments thereof, with at least one scFv that binds to an effector cell surface molecule or surface triggering receptor, and at least another scFv that binds to a target cell via a target cell specific surface molecule. Examples of engagers include, but are not limited to, bi-specific T cell engagers (BiTEs), bi-specific killer cell engagers (BiKEs), tri-specific killer cell engagers (TriKEs), multi-specific killer cell engagers, or universal engagers compatible with multiple immune cell types. Such bi-specific or multi-specific engagers are capable of directing an effector cell (e.g., a T cell, a NK cell, an NKT cell, a B cell, a macrophage, and/or a neutrophil) to a tumor cell and activating the immune effector cell, and have shown great potential to maximize the benefits of CAR-T cell therapy. In some embodiments, the engager expressed by an engineered iPSC-derived effector cell engages a bystander immune cell that comprises a surface molecule recognized and bound by the engager. In some embodiments, the engager expressed by an engineered iPSC-derived effector cell comprising a CAR binds to the derivative effector cell and activates the derivative effector cell upon binding to a tumor antigen different from the CAR antigen. In some embodiments, the engager expressed by an engineered iPSC-derived effector cell comprising a CAR binds to the derivative effector cell through an endogenous surface molecule of the effector cell. In some embodiments, the engager expressed by an engineered iPSC-derived effector cell comprising a CAR binds to the derivative effector cell through an exogenous surface triggering receptor of the effector cell. In some embodiments, the exogenous surface triggering receptor of the effector cell expressing the engager comprises a CFR (chimeric fusion receptor), as further provided herein.

In some embodiments, the surface triggering receptor facilitates bi- or multi-specific antibody engagement between the effector cells and a specific target cell (e.g., a tumor cell), independent of the effector cell's natural receptors and cell type. In some other embodiments, one or more exogenous surface triggering receptors may be introduced to the effector cells using the methods and compositions provided herein, i.e., through engineering of an iPSC, optionally generating a master cell bank comprising single cell sorted and expanded clonal engineered iPSCs, and then directing the differentiation of the iPSC to T, NK or any other effector cells comprising the same genotype as the source iPSC.

Using this approach, one may also generate iPSCs comprising a universal surface triggering receptor, and then differentiate such iPSCs into populations of various effector cell types that express the universal surface triggering receptor. In some embodiments, engagers having the same tumor targeting specificity are used to couple with different universal surface triggering receptors. In some embodiments, engagers having different tumor targeting specificities are used to couple with the same universal surface triggering receptor. As such, one or multiple effector cell types can be engaged to kill one specific type of tumor cells in some cases, and to kill two or more types of tumors in other cases. A surface triggering receptor generally comprises a co-stimulatory domain for effector cell activation and an anti-epitope that is specific to the epitope of an engager, or vice versa, the surface triggering receptor comprises an epitope that is recognizable or specific to the anti-epitope of the engager. For example, a bi-specific engager is specific to the epitope of a surface triggering receptor on one end and is specific to a tumor antigen on the other end.

Exemplary effector cell surface molecules, or surface triggering receptors, that can be used for bi- or multi-specific engager recognition, or coupling, or binding, include, but are not limited to, CD3, CD28, CD5, CD16, CD64, CD32, CD33, CD89, NKG2C, NKG2D, or any functional variants or chimeric receptor forms thereof as disclosed herein. In some embodiments, the CD16 expressed on the surface of effector cells for engager recognition is a hnCD16, comprising CD16 (containing F176V and optionally S197P) or CD64 extracellular domain, and native or non-native transmembrane, stimulatory and/or signaling domains as described herein. In some embodiments, the CD16 expressed on the surface of effector cells for engager recognition is a CD16-based chimeric Fc receptor (CFcR). In some embodiments, the CD16-based CFcR comprises a transmembrane domain of NKG2D, a stimulatory domain of 2B4, and a signaling domain of CD3ζ; wherein the extracellular domain of the CD16 is derived from a full length or partial sequence of the extracellular domain of CD64 or CD16; and optionally wherein the extracellular domain of CD16 comprises F176V and optionally S197P.

Exemplary tumor cell surface molecules for bi- or multi-specific engager recognition include, but are not limited to, B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, and ROR1. In one embodiment, the bi-specific engager is a bi-specific antibody specific to CD3 and CD19 (CD3×CD19 or CD3-CD19); and in another embodiment, the bi-specific antibody is CD3-CD33. In another embodiment, the bi-specific antibody is CD3-EpCAM. For engaging CD16 on an effector cell, the bi-specific antibody comprises CD16-CD30 or CD64-CD30. In another embodiment, the bi-specific antibody comprises CD16-BCMA or CD64-BCMA. In yet another embodiment, the bi-specific antibody further comprises a linker between the effector cell and tumor cell antigen binding domains, for example, a modified IL15 may be used as a linker for effector NK cells to facilitate effector cell expansion/autonomy (called TriKE, or Tri-specific Killer Engager, in some publications). In one embodiment, the TriKE is CD16-IL15-EpCAM or CD64-IL15-EpCAM. In another embodiment, the TriKE is CD16-IL15-B7H3, or CD64-IL15-B7H3. In another embodiment, the TriKE is CD16-IL15-CD33 or CD64-IL15-CD33. In yet another embodiment, the TriKE is NKG2C-IL15-CD33. The IL15 in the TriKE may also originate from other cytokines including, but not limited to, IL2, IL4, IL6, IL7, IL9, IL10, IL11, IL12, IL18, and IL21.

In some embodiments, the engager comprises a first binding domain specific to any one of ADGRE2, B7H3, carbonic anhydrase IX (CAIX), CCR1, CCR4, carcinoembryonic antigen (CEA), CD3, CD5, CD7, CD8, CD10, CD19, CD20, CD22, CD30, CD33, CD34, CD38, CD41, CD44, CD44V6, CD49f, CD52, CD56, CD70, CD74, CD99, CD123, CD133, CD138, CD269 (BCMA), CDS, CLEC12A, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A1 (MAGE-A1), MICA/B, MR1, Mucin 1 (Muc-1), Mucin 16 (Muc-16), Mesothelin (MSLN), NKCSI, NKG2D ligands, c-Met, cancer-testis antigen NYESO1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBCI, TRBC2, vascular endothelial growth factor R2 (VEGF-R2), Wilms tumor protein (WT-1), and various pathogen antigens.

In some embodiments, the surface triggering receptor for bi- or multi-specific engagers could be endogenous to the effector cells, sometimes depending on the cell types. In some other embodiments, one or more exogenous surface triggering receptors could be introduced to the effector cells using the methods and compositions provided herein, i.e., through additional engineering of an iPSC comprising a genotype listed in Table 1, then directing the differentiation of the iPSC to effector cells comprising the same genotype and the surface triggering receptor as the source iPSC.

In some embodiments, as an alternative to the genomically engineered effector cells expressing an engager as provided herein, the engager may be used as a therapeutic agent together with effector cells in a combinational therapy composition targeting one or more antigens associated with a condition, a disease, or an indication (as described above). In some embodiments, the engager is a bi-specific T cell engager (BiTE). In some embodiments, the engager is a bi-specific killer cell engager (BiKE). In some embodiments, the engager is a tri-specific killer cell engager (TriKE). In some embodiments, the engager is a multi-specific killer cell engager. In some embodiments, the engager is a universal engager compatible with multiple immune cell types. In some embodiments, the engager in the combinational therapy or composition used therefor activates bystander immune cells for tumor killing in a recipient of the composition. In some embodiments, the engager in the combinational therapy or composition used therefor activates the effector cells comprised in the combinational therapy composition. In some embodiments of a combinational therapy composition useful for treating liquid or solid tumors, the composition comprises iPSC-derived effector cells comprising at least a CAR, as provided herein. In some embodiments, the iPSC-derived effector cells of the composition comprise hematopoietic lineage cells comprising a genotype listed in Table 1. In some embodiments, the iPSC-derived effector cells of the composition comprise NK lineage cells comprising a genotype listed in Table 1. In some embodiments, the iPSC-derived effector cells of the composition comprise T lineage cells comprising a genotype listed in Table 1.

As such, in addition to genetically engineered immune cells comprising the functional modality as provided herein, embodiments of the present invention provide iPSC-derived effector cells comprising TCR^{exo} and optionally a CAR, and optionally one or more of exogenous CD16 or a variant thereof, TCR^{exo}, or engager, as provided herein. Additionally provided in this application is a master cell bank comprising single cell sorted and expanded clonal engineered iPSCs having at least one phenotype as provided herein, including but not limited to, an engager, wherein the cell bank provides a platform for additional iPSC engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

5. Cell Surface CFR (Chimeric Fusion Receptor)

Implementation of a CFR enables an effector cell to initiate an appropriate signal transduction cascade through CFR binding with a selected agonist for enhancing therapeutic properties of the effector cell expressing the CFR. Such enhanced effector cell therapeutic properties include, but are not limited to, increased activation and cytoxicity, acquired dual targeting capability, prolonged persistency, improved trafficking and tumor penetration, enhanced ability in priming, activating or recruiting bystander immune cells to tumor sites, enhanced ability to resist immunosuppression, improved ability in rescuing tumor antigen escape, and/or controlled cell signaling feedback, metabolism and apoptosis.

Accordingly, in various aspects, the iPSCs and derivative cells comprising TCR^{exo} and optionally one or more of a CAR, exogenous CD16 or a variant thereof, and an engager, may further comprise a CFR that generally comprises an ectodomain fused to a transmembrane domain, which is operatively connected to an endodomain, where the CFR does not have ER (endoplasmic reticulum) retention signals or endocytosis signals in any of the ecto-, transmembrane- or endo-domains. The ectodomain of the CFR is for initiating signal transduction upon binding to an engager; the transmembrane domain is for membrane anchoring of the CFR; and the endodomain comprises at least one signaling domain that regulates (i.e., activates or deactivates) a signaling pathway of choice for enhancing effector cell therapeutic properties including, but not limited to, tumor killing, persistence, mobility, differentiation, TME counteracting, and/or controlled apoptosis. The elimination of ER retention signals from the CFR permits cell surface presentation of the CFR when expressed, while the elimination of endocytosis signals from the CFR reduces CFR internalization and surface downregulation. It is important to either select domain components that have neither ER retention nor endocytosis signals, or remove ER retention or endocytosis signals from selected components of the CFR using molecular engineering tools. In addition, the domains of the CFRs as provided herein are modular, meaning for a given endodomain of a CFR, the ectodomain of the CFR is switchable depending on the binding specificity of a selected agonist, such as an antibody, a BiTE, a TriKE, or any other type of engager, to be used with said CFR; and for a given ectodomain and a specificity matching agonist, the endodomain is switchable depending on the desired signaling pathway to be activated.

In some embodiments, the ectodomain of a CFR described herein comprises a full or partial length of the extracellular portion of a protein that is involved in cell-cell signaling or interactions. In some embodiments, the ectododomain of the CFR comprises a full or partial length of the extracellular portion of CD3ε, CD3γ, CD3δ, CD28, CD5, CD16, CD64, CD32, CD33, CD89, NKG2C, NKG2D, or any functional variants, or combinations and chimerics thereof. In some embodiments, the ectodomain of the CFR is recognized by at least an agonist, for example, an antibody or an engager (e.g., BiTE, BiKE or TriKE), that comprises a binding domain specific to an epitope comprised in the ectodomain of the CFR. In some embodiments, the antibody or engager to be used as an agonist with a CFR-expressing cell binds to at least one extracellular epitope of the CFR, wherein the CFR comprises a full or partial length of the extracellular portion of CD3ε, CD3γ, CD3δ, CD28, CD5, CD16, CD64, CD32, CD33, CD89, NKG2C, NKG2D, or any functional variants or combined/chimeric forms thereof. In some embodiments, the engager recognizes at least one tumor antigen comprising B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1. In particular embodiments, both ER retention and endocytosis signals are absent, or are removed or eliminated, from the CFR ectodomain using genetic engineering methods.

In some embodiments, the ectodomain of the CFR comprises a full or partial length of the extracellular portion of CD3ε, CD3γ, CD3δ or any functional variants or combined/chimeric forms thereof, to utilize a CD3-based agonist. Non-limiting exemplary CD3-based agonists, including but not limited to antibodies or engagers, comprise CD3×CD19, CD3×CD20, CD3×CD33, CD3×EpCAM, CD3×B7H3, blinatumomab, catumaxomab, ertumaxomab, R06958688, AFM11, MT110/AMG 110, MT111/AMG211/MEDI-565, AMG330, MT112/BAY2010112, MOR209/ES414, MGD006/S80880, MGD007, and/or FBTA05. In some embodiments, the ectodomain of the CFR comprises a full or partial length of the extracellular portion of NKG2C, or any functional variants thereof, to utilize an NKG2C-based agonist. Non-limiting exemplary NKG2C-based agonists, including but not limited to antibodies or engagers, comprise NKG2C-IL15-CD33, NKG2C-IL15-CD19, and/or NKG2C-IL15-CD20. In some other embodiments, the ectodomain of the CFR comprises a full or partial length of the extracellular portion of CD28 or any functional variants thereof, to utilize a CD28-based agonist. Non-limiting exemplary CD28-based agonists, including but not limited to antibodies or engagers, comprise at least one of 15E8, CD28.2, CD28.6, YTH913.12, 37.51, 9D7 (TGN1412), 5.11A1, ANC28.1/5D10, and/or 37407.

In some embodiments, the ectodomain of the CFR comprises a full or partial length of the extracellular portion of CD16, CD64, or any functional variants or combined/chimeric forms thereof, to utilize a CD16- or CD64-based agonist. Non-limiting exemplary CD16- or CD64-based agonists, including but not limited to antibodies or engagers, comprise IgG antibodies, or CD16- or CD64-based engagers. When the Fc portion of an IgG antibody binds the CD16- or CD64-based CFRs, it activates antibody dependent cell mediated cytotoxicity (ADCC) in the CFR-expressing cells along with other enhanced therapeutic properties that are imparted by the signaling domains comprised in the endodomains of the CFR. Non-limiting exemplary CD16- or CD64-based agonists, including but not limited to antibodies or engagers, comprise at least one of CD16×CD30, CD64×CD30, CD16×BCMA, CD64×BCMA, CD16-IL-B7H3, CD64-IL-B7H3, CD16-IL-EpCAM or CD64-IL-EpCAM, CD16-IL-CD33 or CD64-IL-CD33, wherein “IL” comprised in a TriKE comprises all or a portion of at least one cytokine comprising IL2, IL4, IL6, IL7, IL9, IL10, IL11, IL12, IL15, IL18, IL21, or any functional variants or combined/chimeric forms thereof.

In general, a transmembrane domain is a three-dimensional protein structure which is thermodynamically stable in a membrane such as the phospholipid bilayer of a biological membrane (e.g., a membrane of a cell or cell vesicle). Thus, in some embodiments, the transmembrane domain of a CFR of the present invention comprises a single alpha helix, a stable complex of several transmembrane alpha helices, a transmembrane beta barrel, a beta-helix of gramicidin A, or any combination thereof. In various embodiments, the transmembrane domain of the CFR comprises all or a portion of a “transmembrane protein” or “membrane protein” that is within the membrane. As used herein, a “transmembrane protein” or “membrane protein” is a protein located at and/or within a membrane. Examples of transmembrane proteins that are suitable for providing a transmembrane domain comprised in a CFR of the invention include, but are not limited to, a receptor, a ligand, an immunoglobulin, a glycophorin, or a combination thereof. In some embodiments, the transmembrane domain comprised in the CFR comprises all or a portion of a transmembrane domain of 2B4, 4-1BB, BTLA, CD2, CD3δ, CD3ε, CD3γ, CD3ζ, CD4, CD8, CD8a, CD8b, CD16, CD27, CD28, CD28H, CD40, CD84, CD166, CS1, CTLA-4, DNAM1, DAP10, DAP12, FcERIγ, ICOS, ICAM-1, IL7, IL12, IL15, KIR2DL4, KIR2DS1, KIR2DS2, LAG3, PD1, NKp30, NKp44, NKp46, NKG2C, NKG2D, OX40, T cell receptor polypeptide (such as TCRα and/or TCRβ), a nicotinic acetylcholine receptor, a GABA receptor, or any combination thereof.

In some embodiments, the transmembrane domain comprises all or a portion of a transmembrane domain of IgG, IgA, IgM, IgE, IgD, or any combination thereof. In some embodiments, the transmembrane domain comprises all or a portion of a transmembrane domain of glycophorin A, glycophorin D or any combination thereof. In particular embodiments of the CFR transmembrane domain, both ER retention and endocytosis signals are absent or are removed using genetic engineering. In various embodiments, both ER retention and endocytosis signals are absent or are removed or eliminated from the CFR transmembrane domain using genetic engineering methods. In some embodiments, the transmembrane domain comprises all or a portion of a transmembrane domain of CD28, CD8, or CD4.

In some embodiments, the endodomain of a CFR comprises at least one signaling domain that activates an intracellular signaling pathway of choice. In various embodiments of the CFR endodomain, both ER retention and endocytosis signals are absent or are removed or eliminated therefrom using genetic engineering methods. In some embodiments, the endodomain comprises at least a cytoxicity domain. In some other embodiments, the endodomain may optionally comprise, in addition to a cytoxicity domain, one or more of a co-stimulatory domain, a persistency signaling domain, a death-inducing signaling domain, a tumor cell control signaling domain, or any combinations thereof. In some embodiments, the signaling peptide of the endodomain (or intracellular domain) comprises a full length or at least a portion of a polypeptide of 2B4, CD2, CD3ζ, CD3 ζ1XX, CD8, CD28, CD28H, CD137 (4-1BB), CS1, DAP10, DAP12, DNAM1, FcERIγ, IL2Rγ, IL7R, IL21R, IL2Rβ (IL15Rβ), IL21, IL7, IL12, IL15, IL21, KIR2DS2, NKp30, NKp44, NKp46, NKG2C, or NKG2D. In some embodiments, the cytoxicity domain of the CFR comprises at least a full length or a portion of a polypeptide of CD3ζ, 2B4, DAP10, DAP12, DNAM1, CD137 (4-1BB), IL21, IL7, IL12, IL15, NKp30, NKp44, NKp46, NKG2C, or NKG2D. In one embodiment, the cytoxicity domain of a CFR comprises an amino acid sequence that has at least about 85%, about 90%, about 95%, about 96%, about 97%, about 98%, or about 99% identity to at least one ITAM (immunoreceptor tyrosine-based activation motif) of CD3ζ. In one embodiment, the cytoxicity domain of the CFR comprises a modified CD3ζ.

In some embodiments, the CFR comprises an endodomain that comprises a co-stimulatory domain in addition to a cytotoxicity signaling domain. Co-stimulatory domains suitable for use in the CFR include, but are not limited to, a full length or at least a portion of a polypeptide of CD2, CD27, CD28, CD40L, 4-1BB, OX40, ICOS, PD-1, LAG-3, 2B4, BTLA, DAP10, DAP12, CTLA-4, or NKG2D, or any combination thereof. In some embodiments, the co-stimulatory domain of the CFR comprises a full length or at least a portion of a polypeptide of CD28, 4-IBB, CD27, CD40L, ICOS, CD2, or combinations thereof. In some embodiments, the CFR comprises an endodomain comprising a co-stimulatory domain of CD28 and a cytoxicity domain of CD3ζ (also referred to as “28ζ”).

In some embodiments, the CFR comprises an endodomain that comprises a persistency signaling domain in addition to a cytotoxicity signaling domain and/or a co-stimulatory domain. Persistency signaling domains suitable for use in the CFR include, but are not limited to, all or a part of an endodomain of a cytokine receptor such as, IL7R, IL15R, IL18R, IL12R, IL23R, or combinations thereof. In additional embodiments, the endodomain of the CFR may comprise a full or a partial intracellular portion of a receptor tyrosine kinase (RTK) such as EGFR to provide tumor cell control, or a tumor necrosis factor receptor (TNFR) such as FAS, to provide controlled cell death ability.

In various embodiments, exemplary CFRs comprise at least one extracellular portion of a CD3 subunit-CD3ε, CD3δ, or CD3γ, or CD28; a transmembrane domain of CD28, CD8, or CD4; and an endodomain of CD3ε, CD3γ, CD3δ, or CD28, with ER retention motifs and/or endocytosis motifs in ecto-, transmembrane, and/or endo-domains eliminated. In various embodiments, a CFR as provided herein further comprises a signal peptide at the N-terminal of the CFR ectodomain.

In some exemplary embodiments, the CFR comprises an ectodomain of one CD3 subunit; in some other embodiments the CFR comprises a single chain heterodimeric ectodomain that comprises the ectodomain of CD3ε linked with that of CD3δ or CD3γ. The linker type and length in the single chain heterodimeric ectodomain may vary.

The cell surface expressed CFR (including CD3-based CFR, also called a cs-CD3 in some contexts) in various constructions as described herein can function as a cell surface triggering receptor for binding with molecules having selected binding specificity, which molecules include antibodies, engagers, and/or CARs. The cell surface expressed CFR of an effector cell may also function with an engager expressed by the effector cell. The cells comprising polynucleotides encoding TCR^{exo} and optionally one or more of a CAR, exogenous CD16 or a variant thereof, an engager, and/or one or more CFRs of the present invention may be any type of cells, including human cells and non-human cells, pluripotent cells or non-pluripotent cells, immune cells or immune regulatory cells, APC (antigen presenting cells) or feeder cells, cells from primary sources (e.g., PMBC), or from cultured or engineered cells (e.g., cell lines, cells, and/or derivative cells differentiated from iPSCs). In some embodiments, the cells comprising polynucleotides encoding TCR^{exo} and optionally one or more of a CAR, exogenous CD16 or a variant thereof, an engager, and/or one or more CFRs comprise primary or derivative CD34⁺ cells, hematopoietic stem and progenitor cells, hematopoietic multipotent progenitor cells, T cell progenitors, NK cell progenitors, T lineage cells, NKT lineage cells, NK lineage cells, or B lineage cells. In some embodiments, the derivative cells comprising polynucleotides encoding TCR^{exo} and optionally one or more of a CAR, exogenous CD16 or a variant thereof, an engager, and/or one or more CFRs are effector cells obtained from differentiating iPSC comprising polynucleotides encoding TCR^{exo} and optionally one or more of the CAR, exogenous CD16 or a variant thereof, an engager, and/or the one or more CFRs. In some embodiments, the derivative effector cells comprising polynucleotides encoding TCR^{exo} and optionally one or more of a CAR, exogenous CD16 or a variant thereof, an engager, and/or one or more CFRs are obtained from engineering the derivative effector cells to incorporate one or more of a CAR, exogenous CD16 or a variant thereof, an engager, and/or the one or more CFRs after generating the derivative effector cells from an iPSC.

As provided further, the cell, or a population thereof, comprising polynucleotides encoding TCR^{exo} and optionally a CAR, optionally an engager, and/or one or more CFRs may further comprise one or more additional engineered modalities described herein and/or as shown in Table 1. Further provided in this application is a master cell bank comprising single cell sorted and expanded clonal engineered iPSCs having at least one phenotype as provided herein, wherein the cell bank provides a platform for additional iPSC engineering and a renewable source for manufacturing off-the-shelf, engineered, homogeneous effector cells, which are well-defined and uniform in composition, and can be mass produced at significant scale in a cost-effective manner.

6. Exogenously Introduced Cytokine and/or Cytokine Signaling

By avoiding systemic high-dose administration of clinically relevant cytokines, the risk of dose-limiting toxicities due to such a practice is reduced while cytokine mediated cell autonomy is being established. To achieve lymphocyte autonomy without the need to additionally administer soluble cytokines, a signaling complex comprising a partial or full length peptide of one or more of IL2, IL4, IL6, IL7, IL9, IL10, IL11, IL12, IL15, IL18, IL21, and/or their respective receptors may be introduced to the cell to enable cytokine signaling with or without the expression of the cytokine itself to achieve lymphocyte autonomy without administered soluble cytokines thereby maintaining or improving cell growth, proliferation, expansion, persistency and/or effector function with reduced risk of cytokine toxicities. In some embodiments, the introduced cytokine and/or its respective native or modified receptor for cytokine signaling (signaling complex) is expressed on the cell surface. In some embodiments, the cytokine signaling is constitutively activated. In some embodiments, the activation of the cytokine signaling is inducible. In some embodiments, the activation of the cytokine signaling is transient and/or temporal.

Various construct designs for introducing a protein complex for signaling of cytokines including, but not limited to, IL2, IL4, IL6, IL7, IL9, IL10, IL11, IL12, IL15, IL18 and IL21, into the cell are provided herein. In embodiments where the signaling complex is for IL15, the transmembrane (TM) domain can be native to the IL15 receptor or may be modified or replaced with a transmembrane domain of any other membrane bound proteins. In some embodiments, IL15 and IL15Rα are co-expressed by using a self-cleaving peptide, mimicking trans-presentation of IL15, without eliminating cis-presentation of IL15. In other embodiments, IL15Rα is fused to IL15 at the C-terminus through a linker, mimicking trans-presentation without eliminating cis-presentation of IL15 as well as ensuring that IL15 is membrane-bound. In other embodiments, IL15Rα with a truncated intracellular domain is fused to IL15 at the C-terminus through a linker, mimicking trans-presentation of IL15, maintaining IL15 membrane-bound, and additionally eliminating cis-presentation and/or any other potential signal transduction pathways mediated by a normal IL15R through its intracellular domain. In yet other embodiments, the cytoplasmic domain of IL15Rα can be omitted without negatively impacting the autonomous feature of the effector cell equipped with IL15. In other embodiments, essentially the entire IL15Rα is removed except for the Sushi domain fused with IL15 at one end and a transmembrane domain on the other (mb-Sushi), optionally with a linker between the Sushi domain and the transmembrane domain. The fused IL15/mb-Sushi is expressed at the cell surface through the transmembrane domain of any membrane bound protein. Thus, unnecessary signaling through IL15Rα, including cis-presentation, is eliminated when only the desirable trans-presentation of IL15 is retained.

In other embodiments, a native or modified IL15Rβ is fused to IL15 at the C-terminus through a linker, enabling constitutive signaling and maintaining IL15 membrane-bound and trans-representation. In other embodiments, a native or modified common receptor γC is fused to IL15 at the C-terminus through a linker for constitutive signaling and membrane bound trans-presentation of the cytokine. The common receptor γC is also called the “common gamma chain” or CD132, which is also known as IL2 receptor subunit gamma or IL2RG. γC is a cytokine receptor subunit that is common to the receptor complexes for many interleukin receptors, including, but not limited to, IL2, IL4, IL7, IL9, IL15 and IL21 receptor. In other embodiments, engineered IL15Rβ that forms a homodimer in the absence of IL15 is useful for producing constitutive signaling of the cytokine.

In embodiments where the signaling complex is for IL7, the transmembrane (TM) domain of any of a variety of designs can be native to the cytokine receptor or may be modified or replaced with a transmembrane domain of any other membrane bound proteins. In some embodiments, a native (or wildtype) or modified IL7R may be fused to IL7 at the C-terminus through a linker, enabling constitutive signaling and maintaining membrane-bound IL7.

In one embodiment, a native or modified common receptor γC, as discussed above, is fused to IL7 at the C-terminus through a linker for constitutive and membrane-bound cytokine signaling complex. In another embodiment, engineered IL7R that forms a homodimer in the absence of IL7 is useful for producing constitutive signaling of the cytokine as well.

As such, in various embodiments, the cytokines IL15 or IL7 and/or their receptors, may be introduced to iPSC using one or more of the construct designs described above, and to its derivative cells upon iPSC differentiation. In addition to an induced pluripotent cell (iPSC), a clonal iPSC, a clonal iPS cell line, or iPSC-derived cells comprising at least one engineered modality as disclosed herein are provided. Also provided is a master cell bank comprising single cell sorted and expanded clonal engineered iPSCs having at least a signaling complex comprising a partial or full peptide of a cell surface expressed exogenous cytokine and/or a receptor thereof, as described in this section, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

In iPSCs and derivative cells therefrom comprising both CAR and exogenous cytokine and/or cytokine receptor signaling (signaling complex, or “IL”), the CAR and IL may be expressed in separate constructs, or may be co-expressed in a bi-cistronic construct comprising both CAR and IL. In some further embodiments, the signaling complex can be linked to either the 5′ or the 3′ end of a CAR expression construct through a self-cleaving 2A coding sequence. As such, an IL signaling complex (e.g., IL7 signaling complex) and CAR may be in a single open reading frame (ORF). In one embodiment, the signaling complex is comprised in CAR-2A-IL or IL-2A-CAR construct. When CAR-2A-IL or IL-2A-CAR is expressed, the self-cleaving 2A peptide allows the expressed CAR and IL to dissociate, and the dissociated IL can then be presented at the cell surface, with the transmembrane domain anchored in the cell membrane. The CAR-2A-IL or IL-2A-CAR bi-cistronic design allows for coordinated CAR and IL signaling complex expression both in timing and quantity, and under the same control mechanism that may be chosen to incorporate, for example, an inducible promoter or promoter with temporal or spatial specificity for the expression of the single ORF. Self-cleaving peptides are found in members of the Picornaviridae virus family, including aphthoviruses such as foot-and-mouth disease virus (FMDV), equine rhinitis A virus (ERAV), Thosea asigna virus (TaV) and porcine tescho virus-1 (PTV-1) (Donnelly, M L, et al, J. Gen. Virol, 82, 1027-101 (2001); Ryan, M D, et al., J. Gen. Virol., 72, 2727-2732 (2001)), and cardioviruses such as Theilovirus (e.g., Theiler's murine encephalomyelitis) and encephalomyocarditis viruses. The 2A peptides derived from FMDV, ERAV, PTV-I, and TaV are sometimes also referred to as “F2A”, “E2A”, “P2A”, and “T2A”, respectively.

The bi-cistronic CAR-2A-IL or IL-2A-CAR embodiment as disclosed herein is also contemplated for expression of any other cytokine or cytokine signaling complex provided herein, for example, IL2, IL4, IL6, IL9, IL10, IL11, IL12, IL18, and IL21. In some embodiments, the bi-cistronic CAR-2A-IL or IL-2A-CAR is for expression of one or more of IL2, IL4, IL7, IL9, IL15 and IL21.

In iPSCs and derivative cells therefrom comprising both TCR^{exo} and optionally a CAR, optionally an engager, and a signaling complex comprising a partial or full peptide of a cell surface expressed exogenous cytokine and/or cytokine receptor thereof, the iPSCs and derivative cells may further comprise one or more of CFR, exogenous CD16 or a variant thereof, CD38 negative, HLA-I and/or HLA-II deficiency, and/or HLA-G.

In some embodiments, the iPSC, and its derivative effector cells comprising any one of the genotypes in Table 1 may additionally comprise deletion or disruption of at least one of TAP1, TAP2, Tapasin, NLRC5, PD1, LAG3, TIM3, RFXANK, RFX5, RFXAP, RAG1, and any gene in the chromosome 6p21 region; or introduction or upregulation of at least one of HLA-E, 4-1BBL, CD4, CD8, CD47, CD113, CD131, CD137, CD80, PDL1, A_{2A}R, TCR, Fc receptor, an antibody, and surface triggering receptor for coupling with bi-specific, multi-specific or universal engagers.

7. HLA-I- and HLA-II-Deficiency

Multiple HLA class I and class II proteins must be matched for histocompatibility in allogeneic recipients to avoid allogeneic rejection problems. Provided herein is an iPSC cell line and its derivative cells differentiated therefrom with eliminated or substantially reduced expression of both HLA class I and HLA class II proteins. HLA class I deficiency can be achieved by functional deletion of any region of the HLA class I locus (chromosome 6p21), or deletion or disruption of HLA class-I associated genes including, but not limited to, beta-2 microglobulin (B2M) gene, TAP1 gene, TAP2 gene and Tapasin. For example, the B2M gene encodes a common subunit essential for cell surface expression of all HLA class I heterodimers. B2M negative cells are HLA-I deficient. HLA class II deficiency can be achieved by functional deletion or disruption of HLA class II associated genes including, but not limited to, RFXANK, CIITA, RFX5 and RFXAP. CIITA is a transcriptional coactivator, functioning through activation of the transcription factor RFX5 required for class II protein expression. CIITA negative cells are HLA-II deficient. Provided herein is an iPSC line and its derivative cells with both HLA-I and HLA-II deficiency, for example lacking both B2M and CIITA expression, wherein the obtained derivative effector cells enable allogeneic cell therapies by eliminating the need for MHC (major histocompatibility complex) matching, and avoiding recognition and killing by host (allogeneic) T cells.

For some cell types, a lack of HLA class I expression leads to lysis by NK cells. To overcome this “missing self” response, HLA-G may be optionally knocked-in to avoid NK cell recognition and killing of the HLA-I deficient effector cells derived from an engineered iPSC. In one embodiment, the HLA-I deficient iPSC and its derivative cells further comprise HLA-G knock-in. In some embodiments, the provided HLA-I deficient iPSC and its derivative cells further comprise one or both of CD58 knockout and CD54 knockout. CD58 (or LFA-3) and CD54 (or ICAM-1) are adhesion proteins initiating signal-dependent cell interactions, and facilitating cell, including immune cell, migration. It was shown that CD58 knockout has a higher efficiency in reducing allogeneic NK cell activation than CD54 knockout; while double knockout of both CD58 and CD54 has the most enhanced reduction of NK cell activation. In some observations, the CD58 and CD54 double knockout is even more effective than HLA-G overexpression for HLA-I deficient cells in overcoming the “missing-self” effect.

As provided herein, in some embodiments, the HLA-I and HLA-II deficient iPSC and its derivative cells have an exogenous polynucleotide encoding HLA-G. In some embodiments, the HLA-I and HLA-II deficient iPSC and its derivative cells are CD58 negative. In some other embodiments, the HLA-I and HLA-II deficient iPSC and its derivative cells are CD54 negative. In yet some other embodiments, the HLA-I and HLA-II deficient iPSC and its derivative cells are CD58 negative and CD54 negative.

Further, in some embodiments of the genetically engineered immune cells, iPSCs and their derivative cells, comprising TCR^{exo} and optionally one or more of a CAR, exogenous CD16 or a variant thereof, one or more CFRs, an engager, and/or a signaling complex comprising a partial or full peptide of a cell surface expressed exogenous cytokine and/or cytokine receptor thereof, the cells are HLA-I and HLA-II deficient and have an exogenous polynucleotide encoding HLA-G. In some embodiments of the genetically engineered immune cells, iPSCs and their derivative cells, comprising TCR^{exo} and optionally one or more of a CAR, exogenous CD16 or a variant thereof, one or more CFRs, an engager, and/or a signaling complex comprising a partial or full peptide of a cell surface expressed exogenous cytokine and/or cytokine receptor thereof, the cells are HLA-I and HLA-II deficient and are CD58 negative. In some embodiments of the genetically engineered immune cells, iPSCs and their derivative cells, comprising TCR^{exo} and optionally one or more of a CAR, exogenous CD16 or a variant thereof, one or more CFRs, an engager, and/or a signaling complex comprising a partial or full peptide of a cell surface expressed exogenous cytokine and/or cytokine receptor thereof, the cells are HLA-I and HLA-II deficient and are CD54 negative. In yet some other embodiments of the genetically engineered immune cells, iPSCs and their derivative cells, comprising TCR^{exo} and optionally one or more of a CAR, exogenous CD16 or a variant thereof, one or more CFRs, an engager, and/or a signaling complex comprising a partial or full peptide of a cell surface expressed exogenous cytokine and/or cytokine receptor thereof, the cells are HLA-I and HLA-II deficient, and are both CD58 negative and CD54 negative.

Additionally provided in this application is a master cell bank comprising single cell sorted and expanded clonal engineered iPSCs having at least one phenotype as provided herein, including but not limited to, HLA-I and/or HLA-II deficiency, wherein the cell bank provides a platform for additional iPSC engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

8. CD38 Knockout

The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Antibody mediated cancer cell depletion is usually attributable to a combination of direct cell apoptosis induction and activation of immune effector mechanisms such as ADCC (antibody-dependent cell-mediated cytotoxicity). In addition to ADCC, the immune effector mechanisms in concert with the therapeutic antibody may also include antibody-dependent cell-mediated phagocytosis (ADCP) and/or complement-dependent cytotoxicity (CDC).

Other than being highly expressed on malignant cells, CD38 is also expressed on plasma cells, as well as on NK cells, and activated T and B cells. During hematopoiesis, CD38 is expressed on CD34⁺ stem cells and lineage-committed progenitors of lymphoid, erythroid, and myeloid, and during the final stages of maturation which continues through the plasma cell stage. As a type II transmembrane glycoprotein, CD38 carries out cell functions as both a receptor and a multifunctional enzyme involved in the production of nucleotide-metabolites. As an enzyme, CD38 catalyzes the synthesis and hydrolysis of the reaction from NAD⁺ to ADP-ribose, thereby producing secondary messengers CADPR and NAADP which stimulate release of calcium from the endoplasmic reticulum and lysosomes, critical for the process of cell adhesion, which process is calcium dependent. As a receptor, CD38 recognizes CD31 and regulates cytokine release and cytotoxicity in activated NK cells. CD38 is also reported to associate with cell surface proteins in lipid rafts, to regulate cytoplasmic Ca′ flux, and to mediate signal transduction in lymphoid and myeloid cells.

In malignancy treatment, systemic use of CD38 antigen binding receptor transduced T cells have been shown to lyse the CD38⁺ fractions of CD34⁺ hematopoietic progenitor cells, monocytes, NK cells, T cells and B cells, leading to incomplete treatment responses and reduced or eliminated efficacy because of the impaired recipient immune effector cell function. In addition, in multiple myeloma patients treated with daratumumab, a CD38-specific antibody, NK cell reduction in both bone marrow and peripheral blood was observed, although other immune cell types, such as T cells and B cells, were unaffected despite their CD38 expression (Casneuf et al., Blood Advances. 2017; 1(23):2105-2114). Without being limited by theories, the present application provides a strategy to leverage the full potential of CD38 targeted cancer treatment by overcoming CD38-specific antibody and/or CD38 antigen binding domain-induced effector cell depletion or reduction through fratricide. In addition, since CD38 is upregulated on activated lymphocytes such as T or B cells, by suppressing activation of these recipient lymphocytes using a CD38-specific antibody, such as daratumumab, in the recipient of allogeneic effector cells, the allorejection against these effector cells would be reduced and/or prevented, thereby increasing effector cell survival and persistency.

As such, the present application also provides a strategy to enhance effector cell persistency and/or survival through reducing or preventing allorejection by using a CD38-specific antibody, a secreted CD38-specific engager or a CD38-CAR (chimeric antigen receptor) against activation of recipient T and B cells, i.e., lymphodepletion of activated T and B cells, often prior to adoptive cell transferring. Specifically, the strategies as provided herein include generating an iPSC line comprising a CD38 knockout, a master cell bank comprising single cell sorted and expanded clonal CD38 negative iPSCs, and obtaining CD38 negative (CD38^{neg}) derivative effector cells through directed differentiation of the engineered iPSC line, wherein the derivative effector cells are protected against fratricide and allorejection among other advantages when CD38 targeted therapeutic moieties are employed with the effector cells. In addition, anti-CD38 monoclonal antibody therapy significantly depletes a patient's activated immune system without adversely affecting the patient's hematopoietic stem cell compartment. A CD38 negative derivative cell has the ability to resist CD38 antibody mediated depletion, and may be effectively administered in combination with an anti-CD38 antibody or CD38-CAR without the use of toxic conditioning agents and thus reduce and/or replace chemotherapy-based lymphodepletion.

In one embodiment as provided herein, the CD38 knockout in an iPSC line is a biallelic knockout. As disclosed herein, the provided CD38 negative iPSC line further comprises at least a TCR^{exo} and optionally a CAR, and optionally one or more of CFR, exogenous CD16 or a variant thereof, and may further comprise one or more additional engineered modalities described herein, and as shown in Table 1; and said iPSC is capable of directed differentiation to produce functional derivative hematopoietic cells including, but not limited to, immune effector cells, including, but not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, common myeloid progenitor cells, common lymphoid progenitor cells, erythrocytes, myeloid cells, neutrophil progenitors, T cells, NKT cells, NK cells, B cells, neutrophils, dendritic cells, macrophages, and derivative immune effector cells having one or more functional features not present in primary NK, T and/or NKT cells. In some embodiments, when an anti-CD38 antibody is used to induce ADCC or an anti-CD38 CAR is used for targeted cell killing, the CD38^{neg} iPSC and/or derivative effector cells thereof are not eliminated by the anti-CD38 antibody, the anti-CD38 CAR, or recipient activated T or B cells, thereby increasing the persistence and/or survival of the iPSC and its effector cell in the presence of, and/or after exposure to, such therapeutic moieties. In some embodiments, the effector cell has increased persistence and/or survival in vivo in the presence of, and/or after exposure to, such therapeutic moieties.

9. Genetically Engineered iPSC Line and iPSC-Derived Cells Provided Herein

1 In light of the above, the present application provides an iPSC, an iPS cell line cell, or a population thereof, and a derivative effector cell obtained from differentiating the iPSC, wherein each cell comprises at least a polynucleotide encoding TCR^{exo} and optionally a CAR, wherein the cell is an eukaryotic cell, an animal cell, a human cell, an induced pluripotent cell (iPSC), an iPSC-derived effector cell, an immune cell, or a feeder cell. Also provided is a master cell bank comprising single cell sorted and expanded clonal engineered iPSCs having a phenotype as described herein, wherein the cell bank provides a renewable source for manufacturing off-the-shelf, engineered, homogeneous cell therapy products, which are well-defined and uniform in composition; and can be mass produced at significant scale in a cost-effective manner. In some embodiments, the iPSC-derived cells are hematopoietic cells including, but not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, and/or sharing features with T cells, NKT cells, NK cells, B cells, neutrophils, dendritic cells, and macrophages. In some embodiments, the iPSC-derived hematopoietic cells comprise immune effector cells expressing at least TCR^{exo} and optionally a CAR. Further provided herein is an iPSC, an iPS cell line cell, or a clonal population thereof, and a derivative functional cell obtained from differentiating the iPSC, wherein each cell comprises a polynucleotide encoding TCR^{exo} and optionally a CAR, and optionally one or more of CFR; an exogenous CD16 or a variant thereof; a cytokine signaling complex comprising a cytokine and/or its receptor or variants thereof; HLA-I deficiency and/or HLA-II deficiency; introduction of HLA-G or non-cleavable HLA-G, or knockout of one or both of CD58 and CD54; and CD38 knockout, wherein the iPSC is capable of directed differentiation to produce functional derivative hematopoietic cells. In some embodiments, the functional derivative hematopoietic cells are immune effector cells. In some embodiments, the functional derivative immune effector cells share features with NK and/or T cells. In some embodiments, the functional derivative immune effector cells sharing features with NK and/or T cells are not NK cells or T cells.

In some embodiments of the iPSC, the iPS cell line cell, or the clonal population thereof, and the derivative functional cell obtained from differentiating the iPSC, wherein each cell comprises at least a polynucleotide encoding a TCR^{exo} and optionally a CAR, the cell is endogenous TCR^{neg}. As used herein, “TCR^{neg}” is also referred to as TCR negative, TCR^{-/-}, “TCR null”, or TCR knockout, which comprises cells without endogenous TCR expression either by nature (for example, NK cell or iPSC-derived NK cell), by gene expression regulation, or by genomic editing of an iPSC cell (for example, iPSC, iPSC reprogrammed from T cell (TiPSC)) or a T cell to knock out an endogenous TCR or one or more subunits thereof, or by obtaining TCR negative derivative cells differentiated from iPSC having TCR knocked out. As such, the TCR that is knocked out in a cell as disclosed herein is an endogenous TCR complex. Disrupting expression of the constant region of either TCRα or TCRβ in a cell is one of many methods of knocking out the endogenous TCR complex of the cell. TCR^{neg} cells are not able to present a CD3 complex to the cell surface despite of the expression of all CD3 subunits in the TCR^{neg} cells, which adversely affects cell functions that require cell surface CD3 recognition, binding and/or signaling. Thus, in some embodiments of the TCR^{neg} cells comprising a polynucleotide encoding CFR, the CFR is CD3-based. In some embodiments, the TCR^{neg} cells which comprise a polynucleotide encoding TCR^{exo} and optionally a CAR also comprise a cell surface CD3 complex, or one or more subunits or subdomains thereof (cs-CD3) when expressed.

In some embodiments, the cell comprising a TCR^{exo}, optionally a CAR and optionally, an engager, also comprises a CAR inserted in a constant region of a TCR. In some embodiments, the cell comprising a TCR^{exo}, optionally a CAR and optionally an engager is TCR^{neg} and comprises a CAR inserted in a constant region of a TCR and the expression of the CAR is driven by an endogenous TCR promoter. In some embodiments, the cell comprising a TCR^{exo}, optionally a CAR and optionally an engager, also comprises an exogenous cytokine signaling of IL2, IL4, IL7, IL9, IL15, IL21, or any combinations thereof. In some embodiments, the exogenous cytokine signaling is cell membrane bound. In some embodiments, the exogenous cytokine signaling comprises an introduced partial or full peptide of a cytokine and/or its respective receptor or mutated or truncated variants thereof. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine signaling is through a retrovirus, Sendai virus, an adenovirus, an episome, mini-circle, or RNAs including mRNA. In some embodiments, the exogenous cell surface cytokine signaling enables IL2 signaling. In some embodiments, the exogenous cell surface cytokine signaling enables IL4 signaling. In some embodiments, the exogenous cell surface cytokine signaling enables IL7 signaling. In some embodiments, the exogenous cell surface cytokine signaling enables IL9 signaling. In some embodiments, the exogenous cell surface cytokine signaling enables IL15 signaling. In some embodiments, the exogenous cell surface cytokine signaling enables IL21 signaling. In some embodiments, the cell comprising a CAR and optionally an engager, further comprises an exogenous CD16 or functional variants or chimeric receptors thereof. In some embodiments, the exogenous CD16 comprises an ectodomain comprising F176V and S197P. In some embodiments, the exogenous CD16 comprises a full or a partial length of an ectodomain of CD64. In some other embodiments, the exogenous CD16 comprises a chimeric Fc receptor. The exogenous CD16 enables cell killing through ADCC, thereby providing a dual targeting mechanism to an effector cell expressing, for example, a CAR.

In some embodiments, the cell comprising a TCR^{exo}, optionally a CAR and optionally an engager, further comprises a CD38 knockout. The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Other than being highly expressed on malignant cells, CD38 is also expressed on plasma cells as well as on NK cells, and activated T and B cells. In some embodiments, effector cells that are CD38^{-/-} can avoid CD38-induced fratricide. In some embodiments, when an anti-CD38 antibody, a CD38 binding CAR, or a CD3 engager comprising anti-CD38 scFV is used to induce the ADCC and/or tumor cell targeting, the CD38^{-/-} iPSC and/or its derivative effector cells can target the CD38 expressing (tumor) cells without causing effector cell elimination, i.e., reduction or depletion of CD38 expressing effector cells, thereby increasing the iPSC and its effector cell persistence and/or survival.

In some embodiments of the cell comprising a polynucleotide encoding a TCR^{exo}, optionally a CAR and optionally, a polynucleotide encoding an engager, the cell further comprises HLA-I and/or HLA-II deficiency (e.g., a B2M knockout and/or a CIITA knockout), and optionally, a polynucleotide encoding HLA-G or HLA-E. In some embodiments of the cell comprising a polynucleotide encoding a TCR^{exo}, optionally a CAR and optionally, a polynucleotide encoding an engager, the cell further comprises HLA-I and/or HLA-II deficiency (e.g., a B2M knockout and/or a CIITA knockout), and optionally, one or both of CD58 and CD54 knockout.

In view of the above, provided herein is an iPSC comprising a polynucleotide encoding a TCR^{exo}, optionally a CAR and optionally, a polynucleotide encoding an engager, and further optionally one, two, three, or more, or all of: TCR^{neg}, an exogenous CD16 or a variant thereof, a CFR, a signaling complex comprising a cell surface expressed exogenous IL, CD38 knockout, and B2M/CIITA knockout; wherein when B2M is knocked out, a polynucleotide encoding HLA-G, or alternatively, one or both of CD58 and CD54 knockout, is optionally introduced, and wherein the iPSC is capable of directed differentiation to produce functional derivative hematopoietic cells.

As such, the present application provides iPSCs and functional derivative hematopoietic cells thereof, which comprise any one of the following genotypes in Table 1. Also provided herein is a master cell bank comprising single cell sorted and expanded clonal engineered iPSCs comprising any one of the following genotypes in Table 1, i.e., having TCR^{exo}, optionally a CAR and one or both of an engager (“Eg” in Table 1) and a CFR, and optionally, one or more of an exogenous CD16 or a variant thereof, a signaling complex comprising a cell surface expressed exogenous IL, CD38 knockout, and HLA-I and/or HLA-II deficiency, without adversely impacting the differentiation potential of the iPSC and function of the derived effector cells. Said cell bank provides a platform for additional iPSC engineering, and a renewable source for manufacturing off-the-shelf, engineered, homogeneous cell therapy products. Depending on the insertion site of any one of the exogenous polynucleotides, the engineered effector cell may be negative in endogenous TCR expression. Furthermore, if the engineered effector cell is of an NK cell lineage, the cell is also TCR negative.

“IL”, as provided in Table 1 stands for one of IL2, IL4, IL6, IL7, IL9, IL10, IL11, IL12, IL15, IL18, and IL21, depending on which specific cytokine/receptor or combination expression is selected; and when IL7 is selected, IL stands for IL7, including IL7Rα and IL7Rβ. Likewise, when IL15 is selected, IL stands for IL15, including IL15Rα and IL15Rβ. In some embodiments, the cell surface expressed exogenous cytokine and/or a receptor thereof comprises at least one of co-expression of IL15 and IL15Rα by using a self-cleaving peptide, a fusion protein of IL15 and IL15Rα, an IL15/IL15Rα fusion protein with intracellular domain of IL15Rα truncated or eliminated, a fusion protein of IL15 and IL15Rβ, a fusion protein of IL15 and common receptor γC, wherein the common receptor γC is native or modified, and a homodimer of IL15Rβ.

In some embodiments, the cell surface expressed exogenous cytokine and/or a receptor thereof comprises at least one of co-expression of IL7 and IL7Rα by using a self-cleaving peptide, a fusion protein of IL7 and IL7Rα, an IL7/IL7Rα fusion protein with intracellular domain of IL7Rα truncated or eliminated, a fusion protein of IL7 and IL7Rβ, a fusion protein of IL7 and common receptor γC, wherein the common receptor γC is native or modified, and a homodimer of IL7Rβ.

Further, when iPSCs and functional derivative hematopoietic cells thereof have a genotype comprising both CAR and IL, the CAR and IL may optionally be comprised in a bi-cistronic expression cassette comprising a 2A sequence. As comparison, in some other embodiments, CAR and IL are in separate expression cassettes comprised in iPSCs and functional derivative hematopoietic cells thereof.

TABLE 1
Applicable Genotypes of the Cells Provided:

| | | | | | | | HLA-G or | |
|---|---|---|---|---|---|---|---|---|
| | | Engager | | | | | (CD58^{-/-} | |
| | | and/or | | | | B2M^{-/-} | w/or w/o | |
| TCR^{exo} | CAR | CFR | CD16 | IL | CD38^{-/-} | CIITA^{-/-} | CD54^{-/-}) | Genotype |
| √ | | | | | | | | 1. TCR^{exo} |
| √ | √ | | | | | | | 2. TCR^{exo} CAR |
| √ | | √ | | | | | | 3. TCR^{exo} Eg |
| | | | | | | | | 4. TCR^{exo} CFR |
| | | | | | | | | 5. TCR^{exo} Eg CFR |
| √ | | | √ | | | | | 6. TCR^{exo} CD16 |
| √ | | | | √ | | | | 7. TCR^{exo} IL |
| √ | | | | | √ | | | 8. TCR^{exo} CD38^{-/-} |
| √ | | | | | | √ | | 9. TCR^{exo} B2M^{-/-}CIITA^{-/-} |
| √ | | | | | | √ | √ | 10. TCR^{exo} B2M^{-/-}CIITA^{-/-} CD58^{-/-} |
| | | | | | | | | 11. TCR^{exo} B2M^{-/-}CIITA^{-/-} CD54^{-/-} |
| | | | | | | | | 12. TCR^{exo} B2M^{-/-}CIITA^{-/-} CD58^{-/-} CD54^{-/-} |
| | | | | | | | | 13. TCR^{exo} B2M^{-/-}CIITA^{-/-} HLA-G |
| √ | √ | √ | | | | | | 14. TCR^{exo} CAR Eg |
| | | | | | | | | 15. TCR^{exo} CAR CFR |
| | | | | | | | | 16. TCR^{exo} CAR Eg CFR |
| √ | √ | | √ | | | | | 17. TCR^{exo} CAR CD16 |
| √ | √ | | | √ | | | | 18. TCR^{exo} CAR IL |
| √ | √ | | | | √ | | | 19. TCR^{exo} CAR CD38^{-/-} |
| √ | √ | | | | | √ | | 20. TCR^{exo} CAR B2M^{-/-}CIITA^{-/-} |
| √ | √ | | | | | √ | √ | 21. TCR^{exo} CAR B2M^{-/-}CIITA^{-/-} CD58^{-/-} |
| | | | | | | | | 22. TCR^{exo} CAR B2M^{-/-}CIITA^{-/-} CD54^{-/-} |
| | | | | | | | | 23. TCR^{exo} CAR B2M^{-/-}CIITA^{-/-} CD58^{-/-} CD54^{-/-} |
| | | | | | | | | 24. TCR^{exo} CAR B2M^{-/-}CIITA^{-/-} HLA-G |
| √ | | √ | √ | | | | | 25. TCR^{exo} Eg CD16 |
| | | | | | | | | 26. TCR^{exo} CFR CD16 |
| | | | | | | | | 27. TCR^{exo} Eg CFR CD16 |
| √ | | √ | | √ | | | | 28. TCR^{exo} Eg IL |
| | | | | | | | | 29. TCR^{exo} CFR IL |
| | | | | | | | | 30. TCR^{exo} Eg CFR IL |
| √ | | √ | | | √ | | | 31. TCR^{exo} Eg CD38^{-/-} |
| | | | | | | | | 32. TCR^{exo} CFR CD38^{-/-} |
| | | | | | | | | 33. TCR^{exo} Eg CFR CD38^{-/-} |
| √ | | √ | | | | √ | | 34. TCR^{exo} Eg B2M^{-/-}CIITA^{-/-} |
| | | | | | | | | 35. TCR^{exo} CFR B2M^{-/-}CIITA^{-/-} |
| | | | | | | | | 36. TCR^{exo} Eg CFR B2M^{-/-}CIITA^{-/-} |
| √ | | √ | | | | √ | √ | 37. TCR^{exo} Eg B2M^{-/-}CIITA^{-/-} CD58^{-/-} |
| | | | | | | | | 38. TCR^{exo} CFR B2M^{-/-}CIITA^{-/-} CD54^{-/-} |
| | | | | | | | | 39. TCR^{exo} Eg CFR B2M^{-/-}CIITA^{-/-} CD58^{-/-} CD54^{-/-} |
| | | | | | | | | 40. TCR^{exo} Eg CFR B2M^{-/-}CIITA^{-/-} HLA-G |
| √ | | | √ | √ | | | | 41. TCR^{exo} CD16 IL |
| √ | | | √ | | √ | | | 42. TCR^{exo} CD16 CD38^{-/-} |
| √ | | | √ | | | √ | | 43. TCR^{exo} CD16 B2M^{-/-}CIITA^{-/-} |
| √ | | | √ | | | √ | √ | 44. TCR^{exo} CD16 B2M^{-/-}CIITA^{-/-} CD58^{-/-} |
| | | | | | | | | 45. TCR^{exo} CD16 B2M^{-/-}CIITA^{-/-} CD54^{-/-} |
| | | | | | | | | 46. TCR^{exo} CD16 B2M^{-/-}CIITA^{-/-} CD58^{-/-} CD54^{-/-} |
| | | | | | | | | 47. TCR^{exo} CD16 B2M^{-/-}CIITA^{-/-} HLA-G |
| √ | | | | √ | √ | | | 48. TCR^{exo} IL CD38^{-/-} |
| √ | | | | √ | | √ | | 49. TCR^{exo} IL B2M^{-/-}CIITA^{-/-} |
| √ | | | | √ | | √ | √ | 50. TCR^{exo} IL B2M^{-/-}CIITA^{-/-} CD58^{-/-} |
| | | | | | | | | 51. TCR^{exo} IL B2M^{-/-}CIITA^{-/-} CD54^{-/-} |
| | | | | | | | | 52. TCR^{exo} IL B2M^{-/-}CIITA^{-/-} CD58^{-/-} CD54^{-/-} |
| | | | | | | | | 53. TCR^{exo} IL B2M^{-/-}CIITA^{-/-} HLA-G |
| √ | | | | | √ | √ | | 54. TCR^{exo} CD38^{-/-} B2M^{-/-}CIITA^{-/-} |
| √ | | | | | √ | √ | √ | 55. TCR^{exo} CD38^{-/-} B2M^{-/-}CIITA^{-/-} CD58^{-/-} |
| | | | | | | | | 56. TCR^{exo} CD38^{-/-} B2M^{-/-}CIITA^{-/-} CD54^{-/-} |
| | | | | | | | | 57. TCR^{exo} CD38^{-/-} B2M^{-/-}CIITA^{-/-} CD58^{-/-} CD54^{-/-} |
| | | | | | | | | 58. TCR^{exo} CD38^{-/-} B2M^{-/-}CIITA^{-/-} HLA-G |
| √ | √ | √ | √ | | | | | 59. TCR^{exo} CAR Eg CD16 |
| | | | | | | | | 60. TCR^{exo} CAR CFR CD16 |
| | | | | | | | | 61. TCR^{exo} CAR Eg CFR CD16 |
| √ | √ | √ | | √ | | | | 62. TCR^{exo} CAR Eg IL |
| | | | | | | | | 63. TCR^{exo} CAR CFR IL |
| | | | | | | | | 64. TCR^{exo} CAR Eg CFR IL |
| √ | √ | √ | | √ | | | | 65. TCR^{exo} CAR Eg CD38^{-/-} |
| | | | | | | | | 66. TCR^{exo} CAR CFR CD38^{-/-} |
| | | | | | | | | 67. TCR^{exo} CAR Eg CFR CD38^{-/-} |
| √ | √ | √ | | | √ | | | 68. TCR^{exo} CAR Eg B2M^{-/-}CIITA^{-/-} |
| | | | | | | | | 69. TCR^{exo} CAR CFR B2M^{-/-}CIITA^{-/-} |
| | | | | | | | | 70. TCR^{exo} CAR Eg CFR B2M^{-/-}CIITA^{-/-} |
| √ | √ | √ | | | √ | √ | | 71. TCR^{exo} CAR Eg B2M^{-/-}CIITA^{-/-} CD58^{-/-} |
| | | | | | | | | 72. TCR^{exo} CAR Eg B2M^{-/-}CIITA^{-/-} CD54^{-/-} |
| | | | | | | | | 73. TCR^{exo} CAR Eg B2M^{-/-}CIITA^{-/-} CD58^{-/-} CD54^{-/-} |
| | | | | | | | | 74. TCR^{exo} CAR Eg B2M^{-/-}CIITA^{-/-} HLA-G |
| | | | | | | | | 75. TCR^{exo} CAR CFR B2M^{-/-}CIITA^{-/-} CD58^{-/-} |
| | | | | | | | | 76. TCR^{exo} CAR CFR B2M^{-/-}CIITA^{-/-} CD54^{-/-} |
| | | | | | | | | 77. TCR^{exo} CAR CFR B2M^{-/-}CIITA^{-/-} CD58^{-/-} CD54^{-/-} |
| | | | | | | | | 78. TCR^{exo} CAR CFR B2M^{-/-}CIITA^{-/-} HLA-G |
| | | | | | | | | 79. TCR^{exo} CAR Eg CFR B2M^{-/-}CIITA^{-/-} CD58^{-/-} |
| | | | | | | | | 80. TCR^{exo} CAR Eg CFR B2M^{-/-}CIITA^{-/-} CD54^{-/-} |
| | | | | | | | | 81. TCR^{exo} CAR Eg CFR B2M^{-/-}CIITA^{-/-} CD58^{-/-} CD54^{-/-} |
| | | | | | | | | 82. TCR^{exo} CAR Eg CFR B2M^{-/-}CIITA^{-/-} HLA-G |
| √ | √ | | √ | √ | | | | 83. TCR^{exo} CAR CD16 IL |
| √ | √ | | √ | | √ | | | 84. TCR^{exo} CAR CD16 CD38^{-/-} |
| √ | √ | | √ | | | √ | | 85. TCR^{exo} CAR CD16 B2M^{-/-}CIITA^{-/-} |
| √ | √ | | √ | | | √ | √ | 86. TCR^{exo} CAR CD16 CD58^{-/-} |
| | | | | | | | | 87. TCR^{exo} CAR CD16 CD54^{-/-} |
| | | | | | | | | 88. TCR^{exo} CAR CD16 CD58^{-/-} CD54^{-/-} |
| | | | | | | | | 89. TCR^{exo} CAR CD16 HLA-G |
| √ | | √ | √ | √ | | | | 90. TCR^{exo} Eg CD16 IL |
| | | | | | | | | 91. TCR^{exo} CFR CD16 IL |
| | | | | | | | | 92. TCR^{exo} Eg CFR CD16 IL |
| √ | | √ | √ | | √ | | | 93. TCR^{exo} Eg CD16 CD38^{-/-} |
| | | | | | | | | 94. TCR^{exo} CFR CD16 CD38^{-/-} |
| | | | | | | | | 95. TCR^{exo} Eg CFR CD16 CD38^{-/-} |
| √ | | √ | √ | | | √ | | 96. TCR^{exo} Eg CD16 B2M^{-/-}CIITA^{-/-} |
| | | | | | | | | 97. TCR^{exo} CFR CD16 B2M^{-/-}CIITA^{-/-} |
| | | | | | | | | 98. TCR^{exo} Eg CFR CD16 B2M^{-/-}CIITA^{-/-} |
| √ | | √ | √ | | | √ | √ | 99. TCR^{exo} Eg CD16 B2M^{-/-}CIITA^{-/-} CD58^{-/-} |
| | | | | | | | | 100. TCR^{exo} Eg CD16 B2M^{-/-}CIITA^{-/-} CD54^{-/-} |
| | | | | | | | | 101. TCR^{exo} Eg CD16 B2M^{-/-}CIITA^{-/-} CD58^{-/-} CD54^{-/-} |
| | | | | | | | | 102. TCR^{exo} Eg CD16 B2M^{-/-}CIITA^{-/-} HLA-G |
| | | | | | | | | 103. TCR^{exo} CFR CD16 B2M^{-/-}CIITA^{-/-} CD58^{-/-} |
| | | | | | | | | 104. TCR^{exo} CFR CD16 B2M^{-/-}CIITA^{-/-} CD54^{-/-} |
| | | | | | | | | 105. TCR^{exo} CFR CD16 B2M^{-/-}CIITA^{-/-} CD58^{-/-} CD54^{-/-} |
| | | | | | | | | 106. TCR^{exo} CFR CD16 B2M^{-/-}CIITA^{-/-} HLA-G |
| | | | | | | | | 107. TCR^{exo} Eg CFR CD16 B2M^{-/-}CIITA^{-/-} CD58^{-/-} |
| | | | | | | | | 108. TCR^{exo} Eg CFR CD16 B2M^{-/-}CIITA^{-/-} CD54^{-/-} |
| | | | | | | | | 109. TCR^{exo} Eg CFR CD16 B2M^{-/-}CIITA^{-/-} CD58^{-/-} CD54^{-/-} |
| | | | | | | | | 110. TCR^{exo} Eg CFR CD16 B2M^{-/-}CIITA^{-/-} HLA-G |
| √ | | √ | | √ | √ | | | 111. TCR^{exo} Eg IL CD38^{-/-} |
| | | | | | | | | 112. TCR^{exo} CFR IL CD38^{-/-} |
| √ | | √ | | √ | | √ | | 113. TCR^{exo} Eg IL B2M^{-/-}CIITA^{-/-} |
| | | | | | | | | 114. TCR^{exo} CFR IL B2M^{-/-}CIITA^{-/-} |
| √ | | √ | | √ | | √ | √ | 115. TCR^{exo} Eg IL B2M^{-/-}CIITA^{-/-} CD58^{-/-} |
| | | | | | | | | 116. TCR^{exo} Eg IL B2M^{-/-}CIITA^{-/-} CD54^{-/-} |
| | | | | | | | | 117. TCR^{exo} Eg IL B2M^{-/-}CIITA^{-/-} CD58^{-/-} CD54^{-/-} |
| | | | | | | | | 118. TCR^{exo} Eg IL B2M^{-/-}CIITA^{-/-} HLA-G |
| | | | | | | | | 119. TCR^{exo} CFR IL B2M^{-/-}CIITA^{-/-} CD58^{-/-} |
| | | | | | | | | 120. TCR^{exo} CFR IL B2M^{-/-}CIITA^{-/-} CD54^{-/-} |
| | | | | | | | | 121. TCR^{exo} CFR IL B2M^{-/-}CIITA^{-/-} CD58^{-/-} CD54^{-/-} |
| | | | | | | | | 122. TCR^{exo} CFR IL B2M^{-/-}CIITA^{-/-} HLA-G |
| √ | | √ | | | √ | √ | | 123. TCR^{exo} Eg CD38^{-/-} B2M^{-/-}CIITA^{-/-} |
| | | | | | | | | 124. TCR^{exo} CFR CD38^{-/-} B2M^{-/-}CIITA^{-/-} |
| | | | | | | | | 125. TCR^{exo} Eg CFR CD38^{-/-} B2M^{-/-}CIITA^{-/-} |
| √ | | √ | | | √ | √ | √ | 126. TCR^{exo} Eg CD38^{-/-} B2M^{-/-}CIITA^{-/-} CD58^{-/-} |
| | | | | | | | | 127. TCR^{exo} Eg CD38^{-/-} B2M^{-/-}CIITA^{-/-} CD54^{-/-} |
| | | | | | | | | 128. TCR^{exo} Eg CD38^{-/-} B2M^{-/-}CIITA^{-/-} CD58^{-/-} CD54^{-/-} |
| | | | | | | | | 129. TCR^{exo} Eg CD38^{-/-} B2M^{-/-}CIITA^{-/-} HLA-G |
| | | | | | | | | 130. TCR^{exo} CFR CD38^{-/-} B2M^{-/-}CIITA^{-/-} CD58^{-/-} |
| | | | | | | | | 131. TCR^{exo} CFR CD38^{-/-} B2M^{-/-}CIITA^{-/-} CD54^{-/-} |
| | | | | | | | | 132. TCR^{exo} CFR CD38^{-/-} B2M^{-/-}CIITA^{-/-} CD58^{-/-} CD54^{-/-} |
| | | | | | | | | 133. TCR^{exo} CFR CD38^{-/-} B2M^{-/-}CIITA^{-/-} HLA-G |
| | | | | | | | | 134. TCR^{exo} Eg CFR CD38^{-/-} B2M^{-/-}CIITA^{-/-} CD58^{-/-} |
| | | | | | | | | 135. TCR^{exo} Eg CFR CD38^{-/-} B2M^{-/-}CIITA^{-/-} CD54^{-/-} |
| | | | | | | | | 136. TCR^{exo} Eg CFR CD38^{-/-} B2M^{-/-}CIITA^{-/-} CD58^{-/-} CD54^{-/-} |
| | | | | | | | | 137. TCR^{exo} Eg CFR CD38^{-/-} B2M^{-/-}CIITA^{-/-} HLA-G |
| √ | | √ | | | √ | √ | | 138. TCR^{exo} Eg CD38^{-/-} B2M^{-/-}CIITA^{-/-} |
| | | | | | | | | 139. TCR^{exo} CFR CD38^{-/-} B2M^{-/-}CIITA^{-/-} |
| | | | | | | | | 140. TCR^{exo} Eg CFR CD38^{-/-} B2M^{-/-}CIITA^{-/-} |
| √ | | √ | | | √ | √ | √ | 141. TCR^{exo} Eg CD38^{-/-} B2M^{-/-}CIITA^{-/-} CD58^{-/-} |
| | | | | | | | | 142. TCR^{exo} Eg CD38^{-/-} B2M^{-/-}CIITA^{-/-} CD54^{-/-} |
| | | | | | | | | 143. TCR^{exo} Eg CD38^{-/-} B2M^{-/-}CIITA^{-/-} CD58^{-/-} CD54^{-/-} |
| | | | | | | | | 144. TCR^{exo} Eg CD38^{-/-} B2M^{-/-}CIITA^{-/-} HLA-G |
| | | | | | | | | 145. TCR^{exo} CFR CD38^{-/-} B2M^{-/-}CIITA^{-/-} CD58^{-/-} |
| | | | | | | | | 146. TCR^{exo} CFR CD38^{-/-} B2M^{-/-}CIITA^{-/-} CD54^{-/-} |
| | | | | | | | | 147. TCR^{exo} CFR CD38^{-/-} B2M^{-/-}CIITA^{-/-} CD58^{-/-} CD54^{-/-} |
| | | | | | | | | 148. TCR^{exo} CFR CD38^{-/-} B2M^{-/-}CIITA^{-/-} HLA-G |
| | | | | | | | | 149. TCR^{exo} Eg CFR CD38^{-/-} B2M^{-/-}CIITA^{-/-} CD58^{-/-} |
| | | | | | | | | 150. TCR^{exo} Eg CFR CD38^{-/-} B2M^{-/-}CIITA^{-/-} CD54^{-/-} |
| | | | | | | | | 151. TCR^{exo} Eg CFR CD38^{-/-} B2M^{-/-}CIITA^{-/-} CD58^{-/-} CD54^{-/-} |
| | | | | | | | | 152. TCR^{exo} Eg CFR CD38^{-/-} B2M^{-/-}CIITA^{-/-} HLA-G |
| √ | | | √ | √ | √ | | | 153. TCR^{exo} CD16 IL CD38^{-/-} |
| √ | | | √ | √ | | √ | | 154. TCR^{exo} CD16 IL B2M^{-/-}CIITA^{-/-} |
| √ | | | √ | √ | | √ | √ | 155. TCR^{exo} CD16 IL B2M^{-/-}CIITA^{-/-} CD58^{-/-} |
| | | | | | | | | 156. TCR^{exo} CD16 IL B2M^{-/-}CIITA^{-/-} CD54^{-/-} |
| | | | | | | | | 157. TCR^{exo} CD16 IL B2M^{-/-}CIITA^{-/-} CD58^{-/-} CD54^{-/-} |
| | | | | | | | | 158. TCR^{exo} CD16 IL B2M^{-/-}CIITA^{-/-} HLA-G |
| √ | | | | √ | √ | √ | | 159. TCR^{exo} IL CD38^{-/-} B2M^{-/-}CIITA^{-/-} |
| √ | | | | √ | √ | √ | √ | 160. TCR^{exo} IL CD38^{-/-} B2M^{-/-}CIITA^{-/-} CD58^{-/-} |
| | | | | | | | | 161. TCR^{exo} IL CD38^{-/-} B2M^{-/-}CIITA^{-/-} CD54^{-/-} |
| | | | | | | | | 162. TCR^{exo} IL CD38^{-/-} B2M^{-/-}CIITA^{-/-} CD58^{-/-} CD54^{-/-} |
| | | | | | | | | 163. TCR^{exo} IL CD38^{-/-} B2M^{-/-}CIITA^{-/-} HLA-G |
| √ | √ | √ | √ | √ | | | | 164. TCR^{exo} CAR Eg CD16 IL |
| | | | | | | | | 165. TCR^{exo} CAR CFR CD16 IL |
| | | | | | | | | 166. TCR^{exo} CAR Eg CFR CD16 IL |
| √ | √ | √ | √ | | √ | | | 167. TCR^{exo} CAR Eg CFR CD16 CD38^{-/-} |
| √ | √ | √ | √ | | | √ | | 168. TCR^{exo} CAR Eg CFR CD16 B2M^{-/-}CIITA^{-/-} |
| √ | √ | √ | √ | | | √ | √ | 169. TCR^{exo} CAR Eg CFR CD16 B2M^{-/-}CIITA^{-/-} CD58^{-/-} |
| | | | | | | | | 170. TCR^{exo} CAR Eg CFR CD16 B2M^{-/-}CIITA^{-/-} CD54^{-/-} |
| | | | | | | | | 171. TCR^{exo} CAR Eg CFR CD16 B2M^{-/-}CIITA^{-/-} CD58^{-/-} CD54^{-/-} |
| | | | | | | | | 172. TCR^{exo} CAR Eg CFR CD16 B2M^{-/-}CIITA^{-/-} HLA-G |
| √ | √ | √ | | √ | √ | | | 173. TCR^{exo} CAR Eg IL CD38^{-/-} |
| | | | | | | | | 174. TCR^{exo} CAR CFR IL CD38^{-/-} |
| | | | | | | | | 175. TCR^{exo} CAR Eg CFR IL CD38^{-/-} |
| √ | √ | √ | | √ | | √ | | 176. TCR^{exo} CAR Eg IL B2M^{-/-}CIITA^{-/-} |
| | | | | | | | | 177. TCR^{exo} CAR CFR IL B2M^{-/-}CIITA^{-/-} |
| | | | | | | | | 178. TCR^{exo} CAR Eg CFR IL B2M^{-/-}CIITA^{-/-} |
| √ | √ | √ | | √ | | √ | √ | 179. TCR^{exo} CAR Eg IL B2M^{-/-}CIITA^{-/-} CD58^{-/-} |
| | | | | | | | | 180. TCR^{exo} CAR Eg IL B2M^{-/-}CIITA^{-/-} CD54^{-/-} |
| | | | | | | | | 181. TCR^{exo} CAR Eg IL B2M^{-/-}CIITA^{-/-} CD58^{-/-} CD54^{-/-} |
| | | | | | | | | 182. TCR^{exo} CAR Eg IL B2M^{-/-}CIITA^{-/-} HLA-G |
| | | | | | | | | 183. TCR^{exo} CAR CFR IL B2M^{-/-}CIITA^{-/-} CD58^{-/-} |
| | | | | | | | | 184. TCR^{exo} CAR CFR IL B2M^{-/-}CIITA^{-/-} CD54^{-/-} |
| | | | | | | | | 185. TCR^{exo} CAR CFR IL B2M^{-/-}CIITA^{-/-} CD58^{-/-} CD54^{-/-} |
| | | | | | | | | 186. TCR^{exo} CAR CFR IL B2M^{-/-}CIITA^{-/-} HLA-G |
| | | | | | | | | 187. TCR^{exo} CAR Eg CFR IL B2M^{-/-}CIITA^{-/-} CD58^{-/-} |
| | | | | | | | | 188. TCR^{exo} CAR Eg CFR IL B2M^{-/-}CIITA^{-/-} CD54^{-/-} |
| | | | | | | | | 189. TCR^{exo} CAR Eg CFR IL B2M^{-/-}CIITA^{-/-} CD58^{-/-} CD54^{-/-} |
| | | | | | | | | 190. TCR^{exo} CAR Eg CFR IL B2M^{-/-}CIITA^{-/-} HLA-G |
| √ | √ | | √ | √ | √ | | | 191. TCR^{exo} CAR CD16 IL CD38^{-/-} |
| √ | √ | | √ | √ | | √ | | 192. TCR^{exo} CAR CD16 IL B2M^{-/-}CIITA^{-/-} |
| √ | √ | | √ | √ | | √ | √ | 193. TCR^{exo} CAR CD16 IL B2M^{-/-}CIITA^{-/-} CD58^{-/-} |
| | | | | | | | | 194. TCR^{exo} CAR CD16 IL B2M^{-/-}CIITA^{-/-} CD54^{-/-} |
| | | | | | | | | 195. TCR^{exo} CAR CD16 IL B2M^{-/-}CIITA^{-/-} CD58^{-/-} CD54^{-/-} |
| | | | | | | | | 196. TCR^{exo} CAR CD16 IL B2M^{-/-}CIITA^{-/-} HLA-G |
| √ | √ | | √ | | √ | √ | | 197. TCR^{exo} CAR CD16 CD38^{-/-} B2M^{-/-}CIITA^{-/-} |
| √ | √ | | √ | | √ | √ | √ | 198. TCR^{exo} CAR CD16 CD38^{-/-} B2M^{-/-}CIITA^{-/-} CD58^{-/-} |
| | | | | | | | | 199. TCR^{exo} CAR CD16 CD38^{-/-} B2M^{-/-}CIITA^{-/-} CD54^{-/-} |
| | | | | | | | | 200. TCR^{exo} CAR CD16 CD38^{-/-} B2M^{-/-}CIITA^{-/-} CD58^{-/-} CD54^{-/-} |
| | | | | | | | | 201. TCR^{exo} CAR CD16 CD38^{-/-} B2M^{-/-}CIITA^{-/-} HLA-G |
| √ | √ | | | √ | √ | √ | | 202. TCR^{exo} CAR IL CCD38^{-/-} B2M^{-/-}CIITA^{-/-} |
| √ | √ | | | √ | √ | √ | √ | 203. TCR^{exo} CAR IL CD38^{-/-} B2M^{-/-}CIITA^{-/-} CD58^{-/-} |
| | | | | | | | | 204. TCR^{exo} CAR IL CD38^{-/-} B2M^{-/-}CIITA^{-/-} CD54^{-/-} |
| | | | | | | | | 205. TCR^{exo} CAR IL CD38^{-/-} B2M^{-/-}CIITA^{-/-} CD58^{-/-} CD54^{-/-} |
| | | | | | | | | 206. TCR^{exo} CAR IL CD38^{-/-} B2M^{-/-}CIITA^{-/-} HLA-G |
| √ | | √ | √ | √ | √ | | | 207. TCR^{exo} Eg CD16 IL CD38^{-/-} |
| | | | | | | | | 208. TCR^{exo} CFR CD16 IL CD38^{-/-} |
| | | | | | | | | 209. TCR^{exo} Eg CFR CD16 IL CD38^{-/-} |
| √ | | √ | √ | √ | | √ | | 210. TCR^{exo} Eg CD16 IL B2M^{-/-}CIITA^{-/-} |
| | | | | | | | | 211. TCR^{exo} CFR CD16 IL B2M^{-/-}CIITA^{-/-} |
| | | | | | | | | 212. TCR^{exo} Eg CFR CD16 IL B2M^{-/-}CIITA^{-/-} |
| √ | | √ | √ | √ | | √ | √ | 213. TCR^{exo} Eg CD16 IL B2M^{-/-}CIITA^{-/-} CD58^{-/-} |
| | | | | | | | | 214. TCR^{exo} Eg CD16 IL B2M^{-/-}CIITA^{-/-} CD54^{-/-} |
| | | | | | | | | 215. TCR^{exo} Eg CD16 IL B2M^{-/-}CIITA^{-/-} CD58^{-/-} CD54^{-/-} |
| | | | | | | | | 216. TCR^{exo} Eg CD16 IL B2M^{-/-}CIITA^{-/-} HLA-G |
| | | | | | | | | 217. TCR^{exo} CFR CD16 IL B2M^{-/-}CIITA^{-/-} CD58^{-/-} |
| | | | | | | | | 218. TCR^{exo} CFR CD16 IL B2M^{-/-}CIITA^{-/-} CD54^{-/-} |
| | | | | | | | | 219. TCR^{exo} CFR CD16 IL B2M^{-/-}CIITA^{-/-} CD58^{-/-} CD54^{-/-} |
| | | | | | | | | 220. TCR^{exo} CFR CD16 IL B2M^{-/-}CIITA^{-/-} HLA-G |
| | | | | | | | | 221. TCR^{exo} Eg CFR CD16 IL B2M^{-/-}CIITA^{-/-} CD58^{-/-} |
| | | | | | | | | 222. TCR^{exo} Eg CFR CD16 IL B2M^{-/-}CIITA^{-/-} CD54^{-/-} |
| | | | | | | | | 223. TCR^{exo} Eg CFR CD16 IL B2M^{-/-}CIITA^{-/-} CD58^{-/-} CD54^{-/-} |
| | | | | | | | | 224. TCR^{exo} Eg CFR CD16 IL B2M^{-/-} CIITA^{-/-} HLA-G |
| √ | | | √ | √ | √ | √ | | 225. TCR^{exo} CD16 IL CD38^{-/-} B2M^{-/-}CIITA^{-/-} |
| √ | | | √ | √ | √ | √ | √ | 226. TCR^{exo} CD16 IL CD38^{-/-} B2M^{-/-}CIITA^{-/-} CD58^{-/-} |
| | | | | | | | | 227. TCR^{exo} CD16 IL CD38^{-/-} B2M^{-/-}CIITA^{-/-} CD54^{-/-} |
| | | | | | | | | 228. TCR^{exo} CD16 IL CD38^{-/-} B2M^{-/-}CIITA^{-/-} CD58^{-/-} CD54^{-/-} |
| | | | | | | | | 229. TCR^{exo} CD16 IL CD38^{-/-} B2M^{-/-}CIITA^{-/-} HLA-G |
| √ | √ | √ | √ | √ | √ | | | 230. TCR^{exo} CAR Eg CD16 IL CD38^{-/-} |
| | | | | | | | | 231. TCR^{exo} CAR CFR CD16 IL CD38^{-/-} |
| | | | | | | | | 232. TCR^{exo} CAR Eg CFR CD16 IL CD38^{-/-} |
| √ | √ | √ | √ | √ | | √ | | 233. TCR^{exo} CAR Eg CD16 IL B2M^{-/-}CIITA^{-/-} |
| | | | | | | | | 234. TCR^{exo} CAR CFR CD16 IL B2M^{-/-}CIITA^{-/-} |
| | | | | | | | | 235. TCR^{exo} CAR Eg CFR CD16 IL B2M^{-/-}CIITA^{-/-} |
| √ | √ | √ | √ | √ | | √ | √ | 236. TCR^{exo} CAR Eg CD16 IL B2M^{-/-}CIITA^{-/-} CD58^{-/-} |
| | | | | | | | | 237. TCR^{exo} CAR Eg CD16 IL B2M^{-/-}CIITA^{-/-} CD54^{-/-} |
| | | | | | | | | 238. TCR^{exo} CAR Eg CD16 IL B2M^{-/-}CIITA^{-/-} CD58^{-/-} CD54^{-/-} |
| | | | | | | | | 239. TCR^{exo} CAR Eg CD16 IL B2M^{-/-}CIITA^{-/-} HLA-G |
| | | | | | | | | 240. TCR^{exo} CAR CFR CD16 IL B2M^{-/-}CIITA^{-/-} CD58^{-/-} |
| | | | | | | | | 241. TCR^{exo} CAR CFR CD16 IL B2M^{-/-}CIITA^{-/-} CD54^{-/-} |
| | | | | | | | | 242. TCR^{exo} CAR CFR CD16 IL B2M^{-/-}CIITA^{-/-} CD58^{-/-} CD54^{-/-} |
| | | | | | | | | 243. TCR^{exo} CAR CFR CD16 IL B2M^{-/-}CIITA^{-/-} HLA-G |
| | | | | | | | | 244. TCR^{exo} CAR Eg CFR CD16 IL B2M^{-/-}CIITA^{-/-} CD58^{-/-} |
| | | | | | | | | 245. TCRex CAR Eg CFR CD16 IL B2M^{-/-}CIITA^{-/-} CD54^{-/-} |
| | | | | | | | | 246. TCR^{exo} CAR Eg CFR CD16 IL B2M^{-/-}CIITA^{-/-} |
| | | | | | | | | CD58^{-/-} CD54^{-/-} |
| | | | | | | | | 247. TCR^{exo} CAR Eg CFR CD16 IL B2M^{-/-}CIITA^{-/-} HLA-G |
| √ | √ | √ | √ | | √ | √ | | 248. TCR^{exo} CAR Eg CD16 CD38^{-/-} B2M^{-/-}CIITA^{-/-} |
| | | | | | | | | 249. TCR^{exo} CAR CFR CD16 CD38^{-/-} B2M^{-/-}CIITA^{-/-} |
| | | | | | | | | 250. TCR^{exo} CAR Eg CFR CD16 CD38^{-/-} B2M^{-/-}CIITA^{-/-} |
| √ | √ | √ | √ | | √ | √ | √ | 251. TCR^{exo} CAR Eg CD16 CD38^{-/-} B2M^{-/-}CIITA^{-/-} CD58^{-/-} |
| | | | | | | | | 252. TCR^{exo} CAR Eg CD16 CD38^{-/-} B2M^{-/-}CIITA^{-/-} CD54^{-/-} |
| | | | | | | | | 253. TCR^{exo} CAR Eg CD16 CD38^{-/-} B2M^{-/-}CIITA^{-/-} |
| | | | | | | | | CD58^{-/-} CD54^{-/-} |
| | | | | | | | | 254. TCR^{exo} CAR Eg CD16 CD38^{-/-} B2M^{-/-}CIITA^{-/-} HLA-G |
| | | | | | | | | 255. TCR^{exo} CAR CFR CD16 CD38^{-/-} B2M^{-/-}CIITA^{-/-} CD58^{-/-} |
| | | | | | | | | 256. TCR^{exo} CAR CFR CD16 CD38^{-/-} B2M^{-/-}CIITA^{-/-} CD54^{-/-} |
| | | | | | | | | 257. TCR^{exo} CAR CFR CD16 CD38^{-/-} B2M^{-/-}CIITA^{-/-} |
| | | | | | | | | CD58^{-/-} CD54^{-/-} |
| | | | | | | | | 258. TCR^{exo} CAR CFR CD16 CD38^{-/-} B2M^{-/-}CIITA^{-/-} HLA-G |
| | | | | | | | | 259. TCR^{exo} CAR Eg CFR CD16 CD38^{-/-} B2M^{-/-}CIITA^{-/-} CD58^{-/-} |
| | | | | | | | | 260. TCR^{exo} CAR Eg CFR CD16 CD38^{-/-} B2M^{-/-}CIITA^{-/-} CD54^{-/-} |
| | | | | | | | | 261. TCR^{exo} CAR Eg CFR CD16 CD38^{-/-} B2M^{-/-}CIITA^{-/-} |
| | | | | | | | | CD58^{-/-} CD54^{-/-} |
| | | | | | | | | 262. TCR^{exo} CAR Eg CFR CD16 CD38^{-/-} B2M^{-/-}CIITA^{-/-} HLA-G |
| √ | √ | √ | | √ | √ | √ | | 263. TCR^{exo} CAR Eg IL CD38^{-/-} B2M^{-/-}CIITA^{-/-} |
| | | | | | | | | 264. TCR^{exo} CAR CFR IL CD38^{-/-} B2M^{-/-}CIITA^{-/-} |
| | | | | | | | | 265. TCR^{exo} CAR Eg CFR IL CD38^{-/-} B2M^{-/-}CIITA^{-/-} |
| √ | √ | √ | | √ | √ | √ | √ | 266. TCR^{exo} CAR Eg IL CD38^{-/-} B2M^{-/-}CIITA^{-/-} CD58^{-/-} |
| | | | | | | | | 267. TCR^{exo} CAR Eg IL CD38^{-/-} B2M^{-/-}CIITA^{-/-} CD54^{-/-} |
| | | | | | | | | 268. TCR^{exo} CAR Eg IL CD38^{-/-} B2M^{-/-}CIITA^{-/-} CD58^{-/-} CD54^{-/-} |
| | | | | | | | | 269. TCR^{exo} CAR Eg IL CD38^{-/-} B2M^{-/-}CIITA^{-/-} HLA-G |
| | | | | | | | | 270. TCR^{exo} CAR CFR IL CD38^{-/-} B2M^{-/-}CIITA^{-/-} CD58^{-/-} |
| | | | | | | | | 271. TCR^{exo} CAR CFR IL CD38^{-/-} B2M^{-/-}CIITA^{-/-} CD54^{-/-} |
| | | | | | | | | 272. TCR^{exo} CAR CFR IL CD38^{-/-} B2M^{-/-}CIITA^{-/-} |
| | | | | | | | | CD58^{-/-} CD54^{-/-} |
| | | | | | | | | 273. TCR^{exo} CAR CFR IL CD38^{-/-} B2M^{-/-}CIITA^{-/-} HLA-G |
| | | | | | | | | 274. TCR^{exo} CAR Eg CFR IL CD38^{-/-} B2M^{-/-}CIITA^{-/-} CD58^{-/-} |
| | | | | | | | | 275. TCR^{exo} CAR Eg CFR IL CD38^{-/-} B2M^{-/-}CIITA^{-/-} CD54^{-/-} |
| | | | | | | | | 276. TCR^{exo} CAR Eg CFR IL CD38^{-/-} B2M^{-/-}CIITA^{-/-} |
| | | | | | | | | CD58^{-/-} CD54^{-/-} |
| | | | | | | | | 277. TCR^{exo} CAR Eg CFR IL CD38^{-/-} B2M^{-/-}CIITA^{-/-} HLA-G |
| √ | √ | | √ | √ | √ | √ | | 278. TCR^{exo} CAR CD16 IL CD38^{-/-} B2M^{-/-}CIITA^{-/-} |
| √ | √ | | √ | √ | √ | √ | √ | 279. TCR^{exo} CAR CD16 IL CD38^{-/-} B2M^{-/-}CIITA^{-/-} CD58^{-/-} |
| | | | | | | | | 280. TCR^{exo} CAR CD16 IL CD38^{-/-} B2M^{-/-}CIITA^{-/-} CD54^{-/-} |
| | | | | | | | | 281. TCR^{exo} CAR CD16 IL CD38^{-/-} B2M^{-/-}CIITA^{-/-} |
| | | | | | | | | CD58^{-/-} CD54^{-/-} |
| | | | | | | | | 282. TCR^{exo} CAR CD16 IL CD38^{-/-} B2M^{-/-}CIITA^{-/-} HLA-G |
| √ | | √ | √ | √ | √ | √ | | 283. TCR^{exo} Eg CD16 IL CD38^{-/-} B2M^{-/-}CIITA^{-/-} |
| | | | | | | | | 284. TCR^{exo} CFR CD16 IL CD38^{-/-} B2M^{-/-}CIITA^{-/-} |
| | | | | | | | | 285. TCR^{exo} Eg CFR CD16 IL CD38^{-/-} B2M^{-/-}CIITA^{-/-} |
| √ | | √ | √ | √ | √ | √ | √ | 286. TCR^{exo} Eg CD16 IL CD38^{-/-} B2M^{-/-}CIITA^{-/-} CD58^{-/-} |
| | | | | | | | | 287. TCR^{exo} Eg CD16 IL CD38^{-/-} B2M^{-/-}CIITA^{-/-} CD54^{-/-} |
| | | | | | | | | 288. TCR^{exo} Eg CD16 IL CD38^{-/-} B2M^{-/-}CIITA^{-/-} CD58^{-/-} CD54^{-/-} |
| | | | | | | | | 289. TCR^{exo} Eg CD16 IL CD38^{-/-} B2M^{-/-}CIITA^{-/-} HLA-G |
| | | | | | | | | 290. TCR^{exo} CFR CD16 IL CD38^{-/-} B2M^{-/-}CIITA^{-/-} CD58^{-/-} |
| | | | | | | | | 291. TCR^{exo} CFR CD16 IL CD38^{-/-} B2M^{-/-}CIITA^{-/-} CD54^{-/-} |
| | | | | | | | | 292. TCR^{exo} CFR CD16 IL CD38^{-/-} B2M^{-/-}CIITA^{-/-} |
| | | | | | | | | CD58^{-/-} CD54^{-/-} |
| | | | | | | | | 293. TCR^{exo} CFR CD16 IL CD38^{-/-} B2M^{-/-}CIITA^{-/-} HLA-G |
| | | | | | | | | 294. TCR^{exo} Eg CFR CD16 IL CD38^{-/-} B2M^{-/-}CIITA^{-/-} CD58^{-/-} |
| | | | | | | | | 295. TCR^{exo} Eg CFR CD16 IL CD38^{-/-} B2M^{-/-}CIITA^{-/-} CD54^{-/-} |
| | | | | | | | | 296. TCR^{exo} Eg CFR CD16 IL CD38^{-/-} B2M^{-/-}CIITA^{-/-} |
| | | | | | | | | CD58^{-/-} CD54^{-/-} |
| | | | | | | | | 297. TCR^{exo} Eg CFR CD16 IL CD38^{-/-} B2M^{-/-}CIITA^{-/-} HLA-G |
| √ | √ | √ | √ | √ | √ | √ | | 298. TCR^{exo} CAR Eg CD16 IL CD38^{-/-} B2M^{-/-}CIITA^{-/-} |
| | | | | | | | | 299. TCR^{exo} CAR CFR CD16 IL CD38^{-/-} B2M^{-/-}CIITA^{-/-} |
| | | | | | | | | 300. TCR^{exo} CAR Eg CFR CD16 IL CD38^{-/-} B2M^{-/-}CIITA^{-/-} |
| √ | √ | √ | √ | √ | √ | √ | √ | 301. TCR^{exo} CAR Eg CD16 IL CD38^{-/-} B2M^{-/-}CIITA^{-/-} CD58^{-/-} |
| | | | | | | | | 302. TCR^{exo} CAR Eg CD16 IL CD38^{-/-} B2M^{-/-}CIITA^{-/-} CD54^{-/-} |
| | | | | | | | | 303. TCR^{exo} CAR Eg CD16 IL CD38^{-/-} B2M^{-/-}CIITA^{-/-} |
| | | | | | | | | CD58^{-/-} CD54^{-/-} |
| | | | | | | | | 304. TCR^{exo} CAR Eg CD16 IL CD38^{-/-} B2M^{-/-}CIITA^{-/-} HLA-G |
| | | | | | | | | 305. TCR^{exo} CAR CFR CD16 IL CD38^{-/-} B2M^{-/-}CIITA^{-/-} CD58^{-/-} |
| | | | | | | | | 306. TCR^{exo} CAR CFR CD16 IL CD38^{-/-} B2M^{-/-}CIITA^{-/-} CD54^{-/-} |
| | | | | | | | | 307. TCR^{exo} CAR CFR CD16 IL CD38^{-/-} B2M^{-/-}CIITA^{-/-} |
| | | | | | | | | CD58^{-/-} CD54^{-/-} |
| | | | | | | | | 308. TCR^{exo} CAR CFR CD16 IL CD38^{-/-} B2M^{-/-}CIITA^{-/-} HLA-G |
| | | | | | | | | 309. TCR^{exo} CAR Eg CFR CD16 IL CD38^{-/-} B2M^{-/-}CIITA^{-/-} |
| | | | | | | | | CD58^{-/-} |
| | | | | | | | | 310. TCR^{exo} CAR Eg CFR CD16 IL CD38^{-/-} B2M^{-/-}CIITA^{-/-} |
| | | | | | | | | CD54^{-/-} |
| | | | | | | | | 311. TCR^{exo} CAR Eg CFR CD16 IL CD38^{-/-} B2M^{-/-}CIITA^{-/-} |
| | | | | | | | | CD58^{-/-} CD54^{-/-} |
| | | | | | | | | 312. TCR^{exo} CAR Eg CFR CD16 IL CD38^{-/-} B2M^{-/-}CIITA^{-/-} |
| | | | | | | | | HLA-G |

10. Antibodies for Immunotherapy

In some embodiments, in addition to the genomically engineered effector cells as provided herein, additional therapeutic agents comprising an antibody, or an antibody fragment thereof, that targets an antigen associated with a condition, a disease, or an indication may be used with these effector cells in a combinational therapy, as compared to being expressed by the genomically engineered effector cells. In some embodiments, the antibody is a monoclonal antibody. In some embodiments, the antibody is a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived effector cells to enhance their killing ability. In some embodiments, the antibodies suitable for combinational treatment as an additional therapeutic agent to the administered iPSC-derived effector cells include, but are not limited to, anti-CD20 (rituximab, veltuzumab, ofatumumab, ublituximab, ocaratuzumab, obinutuzumab, ibritumomab, ocrelizumab), anti-CD22 (inotuzumab, moxetumomab, epratuzumab), anti-HER2 (trastuzumab, pertuzumab), anti-CD52 (alemtuzumab), anti-EGFR (cetuximab), anti-GD2 (dinutuximab), anti-PDL1 (avelumab), anti-CD38 (daratumumab, isatuximab, MOR202), anti-CD123 (7G3, CSL362), anti-SLAMF7 (elotuzumab), and their humanized or Fc modified variants or fragments or their functional equivalents and biosimilars.

In some embodiments, the antibodies suitable for combinational treatment as an additional therapeutic agent to the administered iPSC-derived effector cells further include bi-specific or multi-specific antibodies that target more than one antigen or epitope on a target cell or recruit effector cells (e.g., T cells, NK cells, or macrophage cells) toward target cells while targeting the target cells. Such bi-specific or multi-specific antibodies function as engagers capable of directing an effector cell, whether a bystander immune cell (e.g., a T cell, a NK cell, an NKT cell, a B cell, a macrophage, and/or a neutrophil in the recipient of the therapy) or the engineered effector cell in the therapeutic composition, to a tumor cell and activating the immune effector cell upon binding of the tumor antigen, and have shown great potential to maximize the benefits of antibody therapy. An engager is specific to at least one tumor antigen and is specific to at least one surface triggering receptor of an immune effector cell, which could provide a multi-targeting approach for the engineered cell disclosed herein to address tumor antigen escape and tumor heterogeneity. Examples of engagers include, but are not limited to, bi-specific T cell engagers (BiTEs), bi-specific killer cell engagers (BiKEs), tri-specific killer cell engagers (TriKEs), or multi-specific killer cell engagers, or universal engagers compatible with multiple immune cell types.

In some embodiments, the iPSC-derived effector cells comprise hematopoietic lineage cells comprising a genotype listed in Table 1. In some embodiments, the iPSC-derived effector cells comprise a genotype listed in Table 1. In some embodiments of a combination useful for treating liquid or solid tumors, the combination comprises iPSC-derived effector cells comprising at least a TCR^{exo}, optionally a CAR and optionally a CFR, as provided herein. In some other embodiments of a combination useful for treating liquid or solid tumors, the combination comprises a preselected monoclonal antibody and iPSC-derived effector cells comprising at least a TCR^{exo}, optionally a CAR, and optionally one or more of a CFR and an exogenous CD16 or a variant thereof. In some embodiments of a combination useful for treating liquid or solid tumors, the combination comprises a monoclonal antibody and iPSC-derived effector cells comprising at least a TCR^{exo}, optionally a CAR, and optionally one or more of TCR^{neg}; an exogenous CD16 or a variant thereof; a CFR; an additional cytokine signaling complex comprising a cytokine and/or its receptor or variants thereof; and CD38 knockout. In various embodiments, the exogenous CD16 is hnCD16. Without being limited by the theory, hnCD16 provides enhanced ADCC of the monoclonal antibody, whereas the CAR not only targets a specific tumor antigen but also prevents tumor antigen escape using a dual targeting strategy in combination with a monoclonal antibody targeting a different tumor antigen.

In some further embodiments, the iPSC-derived NK cells comprised in the combination with daratumumab comprise a TCR^{exo}, optionally a CAR, and optionally one or more of exogenous CD16, IL7 or IL15, and the CAR targets at least one of B7H3, MICA/B, CD19, BCMA, CD20, CD22, CD123, HER2, CD52, EGFR, GD2, MSLN, VEGF-R2, PSMA and PDL1; wherein the IL7 or IL15 signaling complex is co- or separately expressed with the CAR.

11. Checkpoint Inhibitors

Checkpoints are cell molecules, often cell surface molecules, capable of suppressing or downregulating immune responses when not inhibited. It is now clear that tumors co-opt certain immune-checkpoint pathways as a major mechanism of immune resistance, particularly against T cells that are specific for tumor antigens. Checkpoint inhibitors (CIs) are antagonists capable of reducing checkpoint gene expression or gene products, or decreasing activity of checkpoint molecules, thereby blocking inhibitory checkpoints and restoring immune system function. The development of checkpoint inhibitors targeting PD1/PDL1 or CTLA4 has transformed the oncology landscape, with these agents providing long term remissions in multiple indications. However, many tumor subtypes are resistant to checkpoint blockade therapy, and relapse remains a significant concern. Thus, one aspect of the present application provides a therapeutic approach to overcome CI resistance by including genomically engineered functional iPSC-derived cells as provided herein in a combination therapy with CI. In one embodiment of the combination therapy, the iPSC-derived cells are NK cells. In another embodiment of the combination therapy, the iPSC-derived cells are T cells. In addition to exhibiting direct antitumor capacity, the derivative NK cells provided herein have been shown to resist PDL1-PD1 mediated inhibition, and to have the ability to enhance T cell migration, to recruit T cells to the tumor microenvironment, and to augment T cell activation at the tumor site. Therefore, the tumor infiltration of T cells facilitated by the functionally potent genomically engineered derivative NK cells indicate that the NK cells are capable of synergizing with T cell targeted immunotherapies, including the checkpoint inhibitors, to relieve local immunosuppression and to reduce tumor burden.

In one embodiment, the iPSC-derived effector cell for checkpoint inhibitor combination therapy comprises a CAR, and optionally one, two, three, four, five or more of: engager expression, exogenous CD16 expression, CFR expression, HLA-I and/or HLA-II deficiency, CD38 knockout, and an exogenous cell surface cytokine and/or receptor expression; wherein when B2M is knocked out, a polynucleotide encoding HLA-G or knockout of one or both of CD58 and CD54 is optionally included. In some embodiments, the derivative NK cell comprises any one of the genotypes listed in Table 1. In some embodiments, the above derivative effector cell additionally comprises deletion or disruption of at least one of TAP1, TAP2, Tapasin, NLRC5, PD1, LAG3, TIM3, RFXANK, RFX5, RFXAP, RAG1, and any gene in the chromosome 6p21 region; or introduction or upregulation of at least one of HLA-E, 4-1BBL, CD3, CD4, CD8, CD47, CD113, CD131, CD137, CD80, PDL1, A_{2A}R, CAR, Fc receptor, and surface triggering receptors for coupling with bi-specific, multi-specific or universal engagers.

In various embodiments, the derivative effector cell is obtained from differentiating an iPSC clonal line comprising one, two, three, four, five or more of: TCR^{exo} expression, CAR expression, engager expression, exogenous CD16 expression, HLA-I and/or HLA-II deficiency, CD38 knockout, and exogenous cell surface cytokine expression; wherein when B2M is knocked out, a polynucleotide encoding HLA-G or knockout of one or both of CD58 and CD54 is optionally introduced. In some embodiments, the above-described iPSC clonal line further comprises deletion or disruption of at least one of TAP1, TAP2, Tapasin, NLRC5, PD1, LAG3, TIM3, RFXANK, RFX5, RFXAP, RAG1, and any gene in the chromosome 6p21 region; or introduction or upregulation of at least one of HLA-E, 4-1BBL, CD3, CD4, CD8, CD47, CD113, CD131, CD137, CD80, PDL1, A_{2A}R, CAR, Fc receptor, and surface triggering receptor for coupling with bi-specific, multi-specific or universal engagers.

Suitable checkpoint inhibitors for combination therapy with the derivative NK or T cells as provided herein include, but are not limited to, antagonists of PD-1 (Pdcd1, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A_{2A}R, BATE, BTLA, CD39 (Entpd1), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a single variable new antigen receptor (VNAR), a shark heavy-chain-only antibody (Ig NAR), chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirimumab (anti-KIR), monalizumab (anti-NKG2A), nivolumab (anti-PD1 mAb), pembrolizumab (anti-PD1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2):103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, and miR-29c.

Some embodiments of the combination therapy with the provided iPSC-derived effector cells comprise at least one checkpoint inhibitor to target at least one checkpoint molecule; wherein the iPSC-derived cells have a genotype listed in Table 1. Some other embodiments of the combination therapy with the provided derivative effector cells comprise two, three or more checkpoint inhibitors such that two, three, or more checkpoint molecules are targeted. In some embodiments of the combination therapy comprising at least one checkpoint inhibitor and the iPSC-derived cells having a genotype listed in Table 1, said checkpoint inhibitor is an antibody, or a humanized or Fc modified variant or fragment, or a functional equivalent or biosimilar thereof, and said checkpoint inhibitor is produced by the iPSC-derived cells by expressing an exogenous polynucleotide sequence encoding said antibody, or a fragment or variant thereof. In some embodiments, the exogenous polynucleotide sequence encoding the antibody, or a fragment or a variant thereof that inhibits a checkpoint is co-expressed with a CAR, either in separate constructs or in a bi-cistronic construct comprising both the CAR and the sequence encoding the antibody, or the fragment thereof. In some further embodiments, the sequence encoding the antibody or the fragment thereof can be linked to either the 5′ or the 3′ end of a CAR expression construct through a self-cleaving 2A coding sequence, illustrated as, for example, CAR-2A-CI or CI-2A-CAR. As such, the coding sequences of the checkpoint inhibitor and the CAR may be in a single open reading frame (ORF). When the checkpoint inhibitor is delivered, expressed and secreted as a payload by the derivative effector cells capable of infiltrating the tumor microenvironment (TME), it counteracts the inhibitory checkpoint molecule upon engaging the TME, allowing activation of the effector cells by activating modalities such as CAR or activating receptors. In some embodiments, the checkpoint inhibitor co-expressed with CAR inhibits at least one of the checkpoint molecules: PD-1, PDL-1, TIM-3, TIGIT, LAG-3, CTLA-4, 2B4, 4-1BB, 4-1BBL, A_{2A}R, BATE, BTLA, CD39 (Entpd1), CD47, CD73 (NTSE), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR. In some embodiments, the checkpoint inhibitor co-expressed with CAR in a derivative cell having a genotype listed in Table 1 is selected from a group comprising atezolizumab, avelumab, durvalumab, tremelimumab, ipilimumab, IPH4102, IPH43, IPH33, lirimumab, monalizumab, nivolumab, pembrolizumab, and their humanized, or Fc modified variants, fragments and their functional equivalents or biosimilars. In some embodiments, the checkpoint inhibitor co-expressed with CAR is atezolizumab, or its humanized, or Fc modified variants, fragments or their functional equivalents or biosimilars. In some other embodiments, the checkpoint inhibitor co-expressed with CAR is nivolumab, or its humanized, or Fc modified variants, fragments or their functional equivalents or biosimilars. In some other embodiments, the checkpoint inhibitor co-expressed with CAR is pembrolizumab, or its humanized, or Fc modified variants, fragments or their functional equivalents or biosimilars.

In some other embodiments of the combination therapy comprising the iPSC-derived cells provided herein and at least one antibody inhibiting a checkpoint molecule, said antibody is not produced by, or in, the iPSC-derived cells and is additionally administered before, with, or after the administering of the iPSC-derived cells having a genotype listed in Table 1. In some embodiments, the administering of one, two, three or more checkpoint inhibitors in a combination therapy with the provided derivative NK or T cells are simultaneous or sequential. In one embodiment of the combination treatment comprising iPSC-derived NK cells or T cells having a genotype listed in Table 1, the checkpoint inhibitor included in the treatment is one or more of atezolizumab, avelumab, durvalumab, tremelimumab, ipilimumab, IPH4102, IPH43, IPH33, lirimumab, monalizuinab, nivolumab, pembrolizumab, and their humanized or Fc modified variants, fragments and their functional equivalents or biosimilars. In some embodiments of the combination treatment comprising iPSC-derived NK cells or T cells having a genotype listed in Table 1, the checkpoint inhibitor included in the treatment is atezolizumab, or its humanized or Fc modified variant, fragment and its functional equivalent or biosimilar. In some embodiments of the combination treatment comprising iPSC-derived NK cells or T cells having a genotype listed in Table 1, the checkpoint inhibitor included in the treatment is nivolumab, or its humanized or Fc modified variant, fragment or its functional equivalent or biosimilar. In some embodiments of the combination treatment comprising derived NK cells or T cells having a genotype listed in Table 1, the checkpoint inhibitor included in the treatment is pembrolizumab, or its humanized or Fc modified variant, fragment or its functional equivalent or biosimilar.

II. Methods for Targeted Genome Editing at Selected Locus in iPSCs

“Genome editing”, or “genomic editing”, or “genetic editing”, as used interchangeably herein, refers to a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. Therefore, targeted editing may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.” In some situations, the targeted integration site is intended to be within a coding region of a selected gene, and thus the targeted integration could disrupt the gene expression, resulting in simultaneous knock-in and knockout (KI/KO) in one single editing step.

Inserting one or more transgenes at a selected position in a gene locus of interest (GOI) to knock out the gene at the same time can be achieved. Gene loci suitable for simultaneous knock-in and knockout (KI/KO) include, but are not limited to, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, and TIGIT. With respective site-specific targeting homology arms for position-selective insertion, it allows the transgene(s) to express either under an endogenous promoter at the site or under an exogenous promoter comprised in the construct. When two or more transgenes are to be inserted at a selected location (e.g., in a CD38 locus), a linker sequence, for example, a 2A linker or IRES, is placed between any two transgenes. The 2A linker encodes a self-cleaving peptide derived from FMDV, ERAV, PTV-I, or TaV (referred to as “F2A”, “E2A”, “P2A”, and “T2A”, respectively), allowing for separate proteins to be produced from a single translation. In some embodiments, insulators are included in the construct to reduce the risk of transgene and/or exogenous promoter silencing. The exogenous promoter may be CAG, or other constitutive, inducible, temporal-, tissue-, or cell type-specific promoters including, but not limited to CMV, EF1α, PGK, and UBC.

Available endonucleases capable of introducing specific and targeted DSBs include, but are not limited to, zinc-finger nucleases (ZFN), transcription activator-like effector nucleases (TALEN), RNA-guided CRISPR (Clustered Regular Interspaced Short Palindromic Repeats) systems. Additionally, DICE (dual integrase cassette exchange) system utilizing phiC31 and Bxb1 integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also International Pub. Nos. WO98/53058; WO98/53059; WO98/53060; WO02/016536 and WO03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the FokI nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Pub. No. 2011/0145940, which is herein incorporated by reference. The most recognized example of a TALEN in the art is a fusion polypeptide of the FokI nuclease to a TAL effector DNA binding domain.

Another example of a targeted nuclease that finds use in the subject methods is a targeted Spo11 nuclease, a polypeptide comprising a Spo11 polypeptide having nuclease activity fused to a DNA binding domain, e.g., a zinc finger DNA binding domain, a TAL effector DNA binding domain, etc., that has specificity for a DNA sequence of interest.

Additional examples of targeted nucleases suitable for the present invention include, but are not limited to, Bxb1, phiC31, R4, PhiBT1, and WP/SPBc/TP901-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

As an exemplary example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. When using the CRISPR/Cpf system, it requires a Cpf endonuclease (Cpf1, MAD7 and many more known in the art) and (2) the gNA, which often does not need tracrRNA, to guide Cpf endonuclease to target selected sequences.

DICE-mediated insertion uses a pair of recombinases, for example, phiC31 and Bxb1, to provide unidirectional integration of an exogenous DNA that is tightly restricted to each enzymes' own small attB and attP recognition sites. Because these target att sites are not naturally present in mammalian genomes, they must be first introduced into the genome at the desired integration site. See, for example, U.S. Pub. No. 2015/0140665, the disclosure of which is incorporated herein by reference.

One aspect of the present invention provides a construct comprising one or more exogenous polynucleotides for targeted genome integration. In one embodiment, the construct further comprises a pair of homologous arms specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Promising sites for targeted integration include, but are not limited to, safe harbor loci, or genomic safe harbor (GSH), which are intragenic or extragenic regions of the human genome that, theoretically, are able to accommodate predictable expression of newly integrated DNA without adverse effects on the host cell or organism. A useful safe harbor must permit sufficient transgene expression to yield desired levels of the vector-encoded protein or non-coding RNA. A safe harbor also must not predispose cells to malignant transformation nor alter cellular functions. For an integration site to be a potential safe harbor locus, it ideally needs to meet criteria including, but not limited to: absence of disruption of regulatory elements or genes, as judged by sequence annotation; is an intergenic region in a gene dense area, or a location at the convergence between two genes transcribed in opposite directions; keep distance to minimize the possibility of long-range interactions between vector-encoded transcriptional activators and the promoters of adjacent genes, particularly cancer-related and microRNA genes; and has apparently ubiquitous transcriptional activity, as reflected by broad spatial and temporal expressed sequence tag (EST) expression patterns, indicating ubiquitous transcriptional activity. This latter feature is especially important in stem cells, where during differentiation, chromatin remodeling typically leads to silencing of some loci and potential activation of others. Within the region suitable for exogenous insertion, a precise locus chosen for insertion should be devoid of repetitive elements and conserved sequences and to which primers for amplification of homology arms could easily be designed.

Suitable sites for human genome editing, or specifically, targeted integration, include, but are not limited to, the adeno-associated virus site 1 (AAVS1), the chemokine (CC motif) receptor 5 (CCR5) gene locus and the human orthologue of the mouse ROSA26 locus. Additionally, the human orthologue of the mouse H11 locus may also be a suitable site for insertion using the composition and method of targeted integration disclosed herein. Further, collagen and HTRP gene loci may also be used as safe harbor for targeted integration. However, validation of each selected site has been shown to be necessary especially in stem cells for specific integration events, and optimization of insertion strategy including promoter election, exogenous gene sequence and arrangement, and construct design is often needed.

For targeted in/dels, the editing site is often comprised in an endogenous gene whose expression and/or function is intended to be disrupted. In one embodiment, the endogenous gene comprising a targeted in/del is associated with immune response regulation and modulation. In some other embodiments, the endogenous gene comprising a targeted in/del is associated with targeting modality, receptors, signaling molecules, transcription factors, drug target candidates, immune response regulation and modulation, or proteins suppressing engraftment, trafficking, homing, viability, self-renewal, persistence, and/or survival of stem cells and/or progenitor cells, and the derived cells therefrom.

As such, another aspect of the present invention provides a method of targeted integration in a selected locus including genome safe harbor or a preselected locus known or proven to be safe and well-regulated for continuous or temporal gene expression such as the B2M, TAP1, TAP2, Tapasin, TRAC, or CD38 locus as provided herein. In one embodiment, the genome safe harbor for the method of targeted integration comprises one or more desired integration sites comprising AAVS1, CCR5, ROSA26, collagen, HTRP, H11, beta-2 microglobulin, CD38, GAPDH, TCR or RUNX1, or other loci meeting the criteria of a genome safe harbor. In some embodiments, the targeted integration is in one or more gene loci where the knock-down or knockout of the gene as a result of the integration is desired, wherein such gene loci include, but are not limited to, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, and TIGIT.

In one embodiment, the method of targeted integration in a cell comprises introducing a construct comprising one or more exogenous polynucleotides to the cell, and introducing a construct comprising a pair of homologous arms specific to a desired integration site and one or more exogenous sequences, to enable site-specific homologous recombination by the cell host enzymatic machinery, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT.

In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or (3 constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT.

Further, as provided herein, the above method for targeted integration in a safe harbor is used to insert any polynucleotide of interest, for example, polynucleotides encoding safety switch proteins, targeting modalities, receptors, signaling molecules, transcription factors, pharmaceutically active proteins and peptides, drug target candidates, and proteins promoting engraftment, trafficking, homing, viability, self-renewal, persistence, and/or survival of stem cells and/or progenitor cells. In some other embodiments, the construct comprising one or more exogenous polynucleotides further comprises one or more marker genes. In one embodiment, the exogenous polynucleotide in a construct of the invention is a suicide gene encoding a safety switch protein. Suitable suicide gene systems for induced cell death include, but are not limited to Caspase 9 (or caspase 3 or 7) and AP1903; thymidine kinase (TK) and ganciclovir (GCV); cytosine deaminase (CD) and 5-fluorocytosine (5-FC). Additionally, some suicide gene systems are cell type specific, for example, the genetic modification of T lymphocytes with the B-cell molecule CD20 allows their elimination upon administration of mAb Rituximab. Further, modified EGFR containing epitope recognized by cetuximab can be used to deplete genetically engineered cells when the cells are exposed to cetuximab. As such, one aspect of the invention provides a method of targeted integration of one or more suicide genes encoding safety switch proteins selected from caspase 9 (caspase 3 or 7), thymidine kinase, cytosine deaminase, modified EGFR, and B-cell CD20.

In some embodiments, one or more exogenous polynucleotides integrated by the method herein are driven by operatively linked exogenous promoters comprised in the construct for targeted integration. The promoters may be inducible, or constitutive, and may be temporal-, tissue- or cell type-specific. Suitable constitutive promoters for methods of the invention include, but are not limited to, cytomegalovirus (CMV), elongation factor 1a (EF1α), phosphoglycerate kinase (PGK), hybrid CMV enhancer/chicken β-actin (CAG) and ubiquitin C (UBC) promoters. In one embodiment, the exogenous promoter is CAG.

The exogenous polynucleotides integrated by the method provided herein may be driven by endogenous promoters in the host genome, at the integration site. In one embodiment, the method of the invention is used for targeted integration of one or more exogenous polynucleotides at the AAVS1 locus in the genome of a cell. In one embodiment, at least one integrated polynucleotide is driven by the endogenous AAVS1 promoter. In another embodiment, the method of the invention is used for targeted integration at the ROSA26 locus in the genome of a cell. In one embodiment, at least one integrated polynucleotide is driven by the endogenous ROSA26 promoter. In still another embodiment, the method of the invention is used for targeted integration at the H11 locus in the genome of a cell. In one embodiment, at least one integrated polynucleotide is driven by the endogenous H11 promoter. In another embodiment, the method of the invention is used for targeted integration at collagen locus in the genome of a cell. In one embodiment, at least one integrated polynucleotide is driven by the endogenous collagen promoter. In still another embodiment, the method of the invention is used for targeted integration at HTRP locus in the genome of a cell. In one embodiment, at least one integrated polynucleotide is driven by the endogenous HTRP promoter. Theoretically, only correct insertions at the desired location would enable gene expression of an exogenous gene driven by an endogenous promoter.

In some embodiments, the one or more exogenous polynucleotides comprised in the construct for the methods of targeted integration are driven by one promoter. In some embodiments, the construct comprises one or more linker sequences between two adjacent polynucleotides driven by the same promoter to provide greater physical separation between the moieties and maximize the accessibility to enzymatic machinery. The linker peptide of the linker sequences may consist of amino acids selected to make the physical separation between the moieties (exogenous polynucleotides, and/or the protein or peptide encoded therefrom) more flexible or more rigid depending on the relevant function. The linker sequence may be cleavable by a protease or cleavable chemically to yield separate moieties. Examples of enzymatic cleavage sites in the linker include sites for cleavage by a proteolytic enzyme, such as enterokinase, Factor Xa, trypsin, collagenase, and thrombin. In some embodiments, the protease is one which is produced naturally by the host or it is exogenously introduced. Alternatively, the cleavage site in the linker may be a site capable of being cleaved upon exposure to a selected chemical or condition, e.g., cyanogen bromide, hydroxylamine, or low pH. The optional linker sequence may serve a purpose other than the provision of a cleavage site. The linker sequence should allow effective positioning of the moiety with respect to another adjacent moiety for the moieties to function properly. The linker may also be a simple amino acid sequence of a sufficient length to prevent any steric hindrance between the moieties. In addition, the linker sequence may provide for post-translational modification including, but not limited to, e.g., phosphorylation sites, biotinylation sites, sulfation sites, γ-carboxylation sites, and the like. In some embodiments, the linker sequence is flexible so as not hold the biologically active peptide in a single undesired conformation. The linker may be predominantly comprised of amino acids with small side chains, such as glycine, alanine, and serine, to provide for flexibility. In some embodiments, about 80 or 90 percent or greater of the linker sequence comprises glycine, alanine, or serine residues, particularly glycine and serine residues. In several embodiments, a G4S linker peptide separates the end-processing and endonuclease domains of the fusion protein. In other embodiments, a 2A linker sequence allows for two separate proteins to be produced from a single translation. Suitable linker sequences can be readily identified empirically. Additionally, suitable sizes and sequences of linker sequences also can be determined by conventional computer modeling techniques. In one embodiment, the linker sequence encodes a self-cleaving peptide. In one embodiment, the self-cleaving peptide is 2A. In some other embodiments, the linker sequence provides an Internal Ribosome Entry Sequence (IRES). In some embodiments, any two consecutive linker sequences are different.

The method of introducing into cells a construct comprising exogenous polynucleotides for targeted integration can be achieved using a method of gene transfer to cells known per se. In one embodiment, the construct comprises backbones of viral vectors such as adenovirus vectors, adeno-associated virus vectors, retrovirus vectors, lentivirus vectors, or Sendai virus vectors. In some embodiments, the plasmid vectors are used for delivering and/or expressing the exogenous polynucleotides to target cells (e.g., pA1-11, pXT1, pRc/CMV, pRc/RSV, pcDNAI/Neo) and the like. In some other embodiments, the episomal vector is used to deliver the exogenous polynucleotide to target cells. In some embodiments, recombinant adeno-associated viruses (rAAVs) can be used for genetic engineering to introduce insertions, deletions or substitutions through homologous recombinations. Unlike lentiviruses, rAAVs do not integrate into the host genome. In addition, episomal rAAV vectors mediate homology-directed gene targeting at much higher rates compared to transfection of conventional targeting plasmids. In some embodiments, an AAV6 or AAV2 vector is used to introduce insertions, deletions or substitutions in a target site in the genome of iPSCs. In some embodiments, the genomically modified iPSCs and their derivative cells obtained using the methods and compositions herein comprise at least one genotype listed in Table 1.

III. Method of Obtaining and Maintaining Genome-Engineered iPSCs

In one aspect, the present invention provides a method of obtaining and maintaining genome-engineered iPSCs comprising one or more targeted edits at one or more desired sites, wherein the targeted edits remain intact and functional in expanded genome-engineered iPSCs or the iPSC-derived non-pluripotent cells at the respective selected editing site(s). The targeted editing introduces into the genome of the iPSC, and derivative cells therefrom, insertions, deletions, and/or substitutions, i.e., targeted integration and/or in/dels at selected sites. In comparison to direct engineering of patient-sourced, peripheral blood originated primary effector cells, the many benefits of obtaining genomically engineered iPSC-derived effector cells through editing and differentiating iPSCs as provided herein include, but are not limited to: unlimited source for engineered effector cells; no need for repeated manipulation of the effector cells especially when multiple engineered modalities are involved; the obtained effector cells are rejuvenated for having elongated telomere and experiencing less exhaustion; the effector cell population is homogeneous in terms of editing site, copy number, and void of allelic variation, random mutations and expression variegation, largely due to the enabled clonal selection in engineered iPSCs as provided herein.

In particular embodiments, the genome-engineered iPSCs comprising one or more targeted edits at one or more selected sites are maintained, passaged and expanded as single cells for an extended period in the cell culture medium shown in Table 2 as Fate Maintenance Medium (FMM), wherein the iPSCs retain the targeted edits and functional modification(s) at the selected site(s). The components of the medium may be present in the medium in amounts within an optimal range shown in Table 2. The iPSCs cultured in FMM have been shown to continue to maintain their undifferentiated, and ground or naïve, profile; genomic stability without the need for culture cleaning or selection; and readily give rise to all three somatic lineages, in vitro differentiation via embryoid bodies or monolayer (without formation of embryoid bodies); and in vivo differentiation by teratoma formation. See, for example, International Pub. No. WO2015/134652, the disclosure of which is incorporated herein by reference.

TABLE 2
Exemplary media for iPSC reprogramming and maintenance

| Conventional hESC | Fate Reprogramming | Fate Maintenance |
|---|---|---|
| Medium (Conv.) | Medium (FRM) | Medium (FMM) |
| DMEM/F12 | DMEM/F12 | DMEM/F12 |
| Knockout | Knockout Serum | Knockout Serum |
| Serum Replacement | Replacement (20%) | Replacement (20%) |
| (20%) | N2 | |
| | B27 | |
| Glutamine | Glutamine | Glutamine (1x) |
| Non-Essential | Non-Essential | Non-Essential |
| Amino Acids (1x) | Amino Acids (1x) | Amino Acids (1x) |
| β-mercaptoethanol (100 μM) | β-mercaptoethanol (100 μM) | β-mercaptoethanol (100 μM) |
| bFGF (0.2-50 ng/mL) | bFGF (2-500 ng/mL) | bFGF (2-500 ng/mL) |
| | LIF (0.2-50 ng/mL) | LIF (0.2-50 ng/mL) |
| | Thiazovivin (0.1-25 μM) | Thiazovivin (0.1-25 μM) |
| | PD0325901 (0.005-2 μM) | PD0325901 (0.005-2 μM) |
| | CHIR99021 (0.02-5 μM) | CHIR99021 (0.02-5 μM) |
| | SB431542 (0.04-10 μM) | |

| | |
|---|---|
| In combination with | Feeder-free, in combination with |
| MEF feeder cells | Matrigel ™ or Vitronectin |

In some embodiments, the genome-engineered iPSCs comprising one or more targeted integration and/or in/dels are maintained, passaged and expanded in a medium comprising a MEK inhibitor, a GSK3 inhibitor, and a ROCK inhibitor, and free of, or essentially free of, TGFβ receptor/ALK5 inhibitors, wherein the iPSCs retain the intact and functional targeted editing at the selected sites.

Another aspect of the invention provides a method of generating genome-engineered iPSCs through targeted editing of iPSCs; or through first generating genome-engineered non-pluripotent cells by targeted editing, and then reprogramming the selected/isolated genome-engineered non-pluripotent cells to obtain iPSCs comprising the same targeted editing as the non-pluripotent cells. A further aspect of the invention provides genome-engineering non-pluripotent cells which are concurrently undergoing reprogramming by introducing targeted integration and/or targeted in/dels to the cells, wherein the contacted non-pluripotent cells are under sufficient conditions for reprogramming, and wherein the conditions for reprogramming comprise contacting non-pluripotent cells with one or more reprogramming factors and small molecules. In various embodiments of the method for concurrent genome-engineering and reprogramming, the targeted integration and/or targeted in/dels may be introduced to the non-pluripotent cells prior to, or essentially concomitantly with, initiating reprogramming by contacting the non-pluripotent cells with one or more reprogramming factors and optionally small molecules.

In some embodiments, to concurrently genome-engineer and reprogram non-pluripotent cells, the targeted integration and/or in/dels may also be introduced to the non-pluripotent cells after the multi-day process of reprogramming is initiated by contacting the non-pluripotent cells with one or more reprogramming factors and small molecules, and wherein the vectors carrying the constructs are introduced before the reprogramming cells present stable expression of one or more endogenous pluripotent genes including, but not limited to, SSEA4, Tra181 and CD30.

In some embodiments, the reprogramming is initiated by contacting the non-pluripotent cells with at least one reprogramming factor, and optionally a combination of a TGFβ receptor/ALK inhibitor, a MEK inhibitor, a GSK3 inhibitor and a ROCK inhibitor (FRM; Table 2). In some embodiments, the genome-engineered iPSCs through any methods above are further maintained and expanded using a mixture of comprising a combination of a MEK inhibitor, a GSK3 inhibitor and a ROCK inhibitor (FMM; Table 2).

In some embodiments of the method of generating genome-engineered iPSCs, the method comprises: genomic engineering an iPSC by introducing one or more targeted integration and/or in/dels into iPSCs to obtain genome-engineered iPSCs having at least one genotype listed in Table 1. Alternatively, the method of generating genome-engineered iPSCs comprises: (a) introducing one or more targeted edits into non-pluripotent cells to obtain genome-engineered non-pluripotent cells comprising targeted integration and/or in/dels at selected sites, and (b) contacting the genome-engineered non-pluripotent cells with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted integration and/or in/dels at selected sites. Alternatively, the method of generating genome-engineered iPSCs comprises: (a) contacting non-pluripotent cells with one or more reprogramming factors, and optionally a small molecule composition comprising a TGFβ receptor/ALK inhibitor, a MEK inhibitor, a GSK3 inhibitor and/or a ROCK inhibitor to initiate the reprogramming of the non-pluripotent cells; (b) introducing one or more targeted integrations and/or in/dels into the reprogramming non-pluripotent cells for genome-engineering; and (c) obtaining genome-engineered iPSCs comprising targeted integration and/or in/dels at selected sites. Any of the above methods may further comprise single cell sorting genome-engineered iPSCs to obtain a clonal iPSC. Through clonal expansion of this genome-engineered iPSC, a master cell bank is generated to comprise single cell sorted and expanded clonal engineered iPSCs having at least one phenotype as provided herein in Table 1. The master cell bank is subsequently cryopreserved, providing a platform for additional iPSC engineering and a renewable source for manufacturing off-the-shelf, engineered, homogeneous cell therapy products, which are 1 volt-defined and uniform in composition, and can be mass produced at significant scale in a cost-effective manner.

The reprogramming factors are selected from the group consisting of OCT4, SOX2, NANOG, KLF4, LIN28, C-MYC, ECAT1, UTF1, ESRRB, SV40LT, HESRG, CDH1, TDGF1, DPPA4, DNMT3B, ZIC3, L1TD1, and any combinations thereof as disclosed in International Pub. Nos. WO2015/134652 and WO2017/066634, the disclosure of which are incorporated herein by reference. The one or more reprogramming factors may be in the form of a polypeptide. The reprogramming factors may also be in the form of polynucleotides, and thus are introduced to the non-pluripotent cells by vectors such as, a retrovirus, a Sendai virus, an adenovirus, an episome, a plasmid, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the one or more polynucleotides introduced by a combination of plasmids. See, for example, International Pub. No. WO2019/075057, the disclosure of which is incorporated herein by reference.

In some embodiments, the non-pluripotent cells are transfected with multiple constructs comprising different exogenous polynucleotides and/or different promoters by multiple vectors for targeted integration at the same or different selected sites. These exogenous polynucleotides may comprise a suicide gene, or a gene encoding targeting modalities, receptors, signaling molecules, transcription factors, pharmaceutically active proteins and peptides, drug target candidates, or a gene encoding a protein promoting engraftment, trafficking, homing, viability, self-renewal, persistence, and/or survival of the iPSCs or derivative cells therefrom. In some embodiments, the exogenous polynucleotides encode RNA, including but not limited to siRNA, shRNA, miRNA and antisense nucleic acids. These exogenous polynucleotides may be driven by one or more promoters selected from the group consisting of constitutive promoters, inducible promoters, temporal-specific promoters, and tissue or cell type specific promoters. Accordingly, the polynucleotides are expressible when under conditions that activate the promoter, for example, in the presence of an inducing agent or in a particular differentiated cell type. In some embodiments, the polynucleotides are expressed in iPSCs and/or in cells differentiated from the iPSCs. In one embodiment, one or more suicide gene is driven by a constitutive promoter, for example Capase-9 driven by CAG. These constructs comprising different exogenous polynucleotides and/or different promoters can be transfected to non-pluripotent cells either simultaneously or consecutively. The non-pluripotent cells subjecting to targeted integration of multiple constructs can simultaneously contact the one or more reprogramming factors to initiate the reprogramming concurrently with the genomic engineering, thereby obtaining genome-engineered iPSCs comprising multiple targeted integration in the same pool of cells. As such, this robust method enables a concurrent reprogramming and engineering strategy to derive a clonal genomically engineered iPSC with multiple modalities integrated to one or more selected target sites. In some embodiments, the genomically modified iPSCs and their derivative cells obtained using the methods and compositions provided herein comprise at least one genotype listed in Table 1.

IV. A Method of Obtaining Genetically-Engineered Effector Cells by Differentiating Genome-Engineered iPSC

A further aspect of the present invention provides a method of in vivo differentiation of genome-engineered iPSC by teratoma formation, wherein the differentiated cells derived in vivo from the genome-engineered iPSCs retain the intact and functional targeted editing including targeted integration and/or in/dels at the desired site(s). In some embodiments, the differentiated cells derived in vivo from the genome-engineered iPSCs via teratoma comprise one or more inducible suicide genes integrated at one or more desired sites comprising AAVS1, CCR5, ROSA26, collagen, HTRP H11, beta-2 microglobulin, CD38, GAPDH, TCR or RUNX1, or other loci meeting the criteria of a genome safe harbor. In some other embodiments, the differentiated cells derived in vivo from the genome-engineered iPSCs via teratoma comprise polynucleotides encoding targeting modality, or encoding proteins promoting trafficking, homing, viability, self-renewal, persistence, and/or survival of stem cells and/or progenitor cells. In some embodiments, the differentiated cells derived in vivo from the genome-engineered iPSCs via teratoma comprising one or more inducible suicide genes further comprise one or more in/dels in endogenous genes associated with immune response regulation and mediation. In some embodiments, the in/del is comprised in one or more endogenous checkpoint genes. In some embodiments, the in/del is comprised in one or more endogenous T cell receptor genes. In some embodiments, the in/del is comprised in one or more endogenous MHC class I suppressor genes. In some embodiments, the in/del is comprised in one or more endogenous genes associated with the major histocompatibility complex. In some embodiments, the in/del is comprised in one or more endogenous genes including, but not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT. In one embodiment, the genome-engineered iPSC comprising one or more exogenous polynucleotides at selected site(s) further comprises targeted editing in a B2M (beta-2-microglobulin) encoding gene.

In particular embodiments, the genome-engineered iPSCs comprising one or more genetic modifications as provided herein are used to derive hematopoietic cell lineages or any other specific cell types in vitro, wherein the derived non-pluripotent cells retain the functional genetic modifications including targeted editing at the selected site(s). In some embodiments, the genome-engineered iPSCs used to derive hematopoietic cell lineages or any other specific cell types in vitro are master cell bank cells that are cryopreserved and thawed right before their usage. In one embodiment, the genome-engineered iPSC-derived cells include, but are not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages, wherein these cells derived from the genome-engineered iPSCs retain the functional genetic modifications including targeted editing at the desired site(s).

Applicable differentiation methods and compositions for obtaining iPSC-derived hematopoietic cell lineages include those depicted in, for example, International Pub. No. WO2017/078807, the disclosure of which is incorporated herein by reference. As provided, the methods and compositions for generating hematopoietic cell lineages are through definitive hemogenic endothelium (HE) derived from pluripotent stem cells, including iPSCs, under serum-free, feeder-free, and/or stromal-free conditions and in a scalable and monolayer culturing platform without the need of EB formation. Cells that may be differentiated according to the provided methods range from pluripotent stem cells, to progenitor cells that are committed to particular terminally differentiated cells and transdifferentiated cells, and to cells of various lineages directly transitioned to hematopoietic fate without going through a pluripotent intermediate. Similarly, the cells that are produced by differentiating stem cells range from multipotent stem or progenitor cells, to terminally differentiated cells, and to all intervening hematopoietic cell lineages.

The methods for differentiating and expanding cells of the hematopoietic lineage from pluripotent stem cells in monolayer culturing comprise contacting the pluripotent stem cells with a BMP pathway activator, and optionally, bFGF. As provided, the pluripotent stem cell-derived mesodermal cells are obtained and expanded without forming embryoid bodies from pluripotent stem cells. The mesodermal cells are then subjected to contact with a BMP pathway activator, bFGF, and a WNT pathway activator to obtain expanded mesodermal cells having definitive hemogenic endothelium (HE) potential without forming embryoid bodies from the pluripotent stem cells. By subsequent contact with bFGF, and optionally, a ROCK inhibitor, and/or a WNT pathway activator, the mesodermal cells having definitive HE potential are differentiated to definitive HE cells, which are also expanded during differentiation.

The methods provided herein for obtaining cells of the hematopoietic lineage are superior to EB-mediated pluripotent stem cell differentiation, because EB formation leads to modest to minimal cell expansion, does not allow monolayer culturing which is important for many applications requiring homogeneous expansion and homogeneous differentiation of the cells in a population, and is laborious and of low efficiency.

The provided monolayer differentiation platform facilitates differentiation towards definitive hemogenic endothelium resulting in the derivation of hematopoietic stem cells and differentiated progeny such as T, B, NKT and NK cells. The monolayer differentiation strategy combines enhanced differentiation efficiency with large-scale expansion and enables the delivery of therapeutically relevant numbers of pluripotent stem cell-derived hematopoietic cells for various therapeutic applications. Further, the monolayer culturing using the methods provided herein leads to functional hematopoietic lineage cells that enable a full range of in vitro differentiation, ex vivo modulation, and in vivo long term hematopoietic self-renewal, reconstitution and engraftment. As provided, the iPSC-derived hematopoietic lineage cells include, but are not limited to, definitive hemogenic endothelium, hematopoietic multipotent progenitor cells, hematopoietic stem and progenitor cells, T cell progenitors, NK cell progenitors, T cells, NK cells, NKT cells, B cells, macrophages, and neutrophils.

In some embodiments, the invention provides a method for directing differentiation of pluripotent stem cells into cells of a definitive hematopoietic lineage, wherein the method comprises: (i) contacting pluripotent stem cells with a composition comprising a BMP activator, and optionally bFGF, to initiate differentiation and expansion of mesodermal cells from the pluripotent stem cells; (ii) contacting the mesodermal cells with a composition comprising a BMP activator, bFGF, and a GSK3 inhibitor, wherein the composition is optionally free of TGFβ receptor/ALK inhibitor, to initiate differentiation and expansion of mesodermal cells having definitive HE potential from the mesodermal cells; (iii) contacting the mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is optionally free of TGFβ receptor/ALK inhibitor, to initiate differentiation and expansion of definitive hemogenic endothelium from pluripotent stem cell-derived mesodermal cells having definitive hemogenic endothelium potential.

In some embodiments, the method further comprises contacting pluripotent stem cells with a composition comprising a MEK inhibitor, a GSK3 inhibitor, and a ROCK inhibitor, wherein the composition is free of TGFβ receptor/ALK inhibitors, to seed and expand the pluripotent stem cells. In some embodiments, the pluripotent stem cells are iPSCs, or naïve iPSCs, or iPSCs comprising one or more genetic imprints; and the one or more genetic imprints comprised in the iPSCs are retained in the hematopoietic cells differentiated therefrom. In some embodiments of the method for directing differentiation of pluripotent stem cells into cells of a hematopoietic lineage, the differentiation of the pluripotent stem cells into cells of hematopoietic lineage is void of generation of embryoid bodies and is in a monolayer culturing form.

In some embodiments of the above method, the obtained pluripotent stem cell-derived definitive hemogenic endothelium cells are CD34⁺. In some embodiments, the obtained definitive hemogenic endothelium cells are CD34⁺CD43⁻. In some embodiments, the definitive hemogenic endothelium cells are CD34⁺CD43⁻CXCR4⁻CD73⁻. In some embodiments, the definitive hemogenic endothelium cells are CD34⁺CXCR4⁻CD73⁻. In some embodiments, the definitive hemogenic endothelium cells are CD34⁺CD43⁻CD93⁻. In some embodiments, the definitive hemogenic endothelium cells are CD34⁺CD93⁻.

In some embodiments of the above method, the method further comprises (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate the differentiation of the definitive hemogenic endothelium to pre-T cell progenitors; and optionally, (ii) contacting the pre-T cell progenitors with a composition comprising one or more growth factors and cytokines selected from the group consisting of SCF, Flt3L, and IL7, but free of one or more of VEGF, bFGF, TPO, BMP activators and ROCK inhibitors, to initiate the differentiation of the pre-T cell progenitors to T cell progenitors or T cells. In some embodiments of the method, the pluripotent stem cell-derived T cell progenitors are CD34⁺CD45⁺CD7⁺. In some embodiments of the method, the pluripotent stem cell-derived T cell progenitors are CD45⁺CD7⁺.

In yet some embodiments of the above method for directing differentiation of pluripotent stem cells into cells of a hematopoietic lineage, the method further comprises: (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15; and optionally, a BMP activator, to initiate differentiation of the definitive hemogenic endothelium to pre-NK cell progenitor; and optionally, (ii) contacting pluripotent stem cells-derived pre-NK cell progenitors with a composition comprising one or more growth factors and cytokines selected from the group consisting of SCF, Flt3L, IL3, IL7, and IL15, wherein the medium is free of one or more of VEGF, bFGF, TPO, BMP activators and ROCK inhibitors, to initiate differentiation of the pre-NK cell progenitors to NK cell progenitors or NK cells. In some embodiments, the pluripotent stem cell-derived NK progenitors are CD3⁻CD45⁺CD56⁺CD7⁺. In some embodiments, the pluripotent stem cell-derived NK cells are CD3⁻CD45⁺CD56⁺, and optionally further defined by NKp46⁺, CD57⁺ and CD16⁺.

Therefore, using the above differentiation methods, one may obtain one or more population of iPSC-derived hematopoietic cells that are: (i) CD34⁺ HE cells (iCD34), using one or more culture medium selected from iMPP-A, iTC-A2, iTC-B2, iNK-A2, and iNK-B2; (ii) definitive hemogenic endothelium (iHE), using one or more culture medium selected from iMPP-A, iTC-A2, iTC-B2, iNK-A2, and iNK-B2; (iii) definitive HSCs, using one or more culture medium selected from iMPP-A, iTC-A2, iTC-B2, iNK-A2, and iNK-B2; (iv) multipotent progenitor cells (iMPP), using iMPP-A; (v) T cell progenitors (ipro-T), using one or more culture medium selected from iTC-A2, and iTC-B2; (vi) T cells (iTC), using iTC-B2; (vii) NK cell progenitors (ipro-NK), using one or more culture medium selected from iNK-A2, and iNK-B2; and/or (viii) NK cells (iNK), and iNK-B2. In some embodiments, the medium:
a. iCD34-C comprises a ROCK inhibitor, one or more growth factors and cytokines selected from the group consisting of bFGF, VEGF, SCF, IL6, IL11, IGF, and EPO, and optionally, a Wnt pathway activator; and is free of TGFβ receptor/ALK inhibitor;
b. iMPP-A comprises a BMP activator, a ROCK inhibitor, and one or more growth factors and cytokines selected from the group consisting of TPO, IL3, GMCSF, EPO, bFGF, VEGF, SCF, IL6, Flt3L and IL11;
c. iTC-A2 comprises a ROCK inhibitor; one or more growth factors and cytokines selected from the group consisting of SCF, Flt3L, TPO, and IL7; and optionally, a BMP activator;
d. iTC-B2 comprises one or more growth factors and cytokines selected from the group consisting of SCF, Flt3L, and IL7;
e. iNK-A2 comprises a ROCK inhibitor, and one or more growth factors and cytokines selected from the group consisting of SCF, Flt3L, TPO, IL3, IL7, and IL15; and optionally, a BMP activator, and
f. iNK-B2 comprises one or more growth factors and cytokines selected from the group consisting of SCF, Flt3L, IL7 and IL15.

In some embodiments, the genome-engineered iPSC-derived cells obtained from the above methods comprise one or more inducible suicide gene integrated at one or more desired integration sites comprising AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT, or other loci meeting the criteria of a genome safe harbor. In some other embodiments, the genome-engineered iPSC-derived cells comprise polynucleotides encoding safety switch proteins, targeting modalities, receptors, signaling molecules, transcription factors, pharmaceutically active proteins and peptides, drug target candidates, or proteins promoting trafficking, homing, viability, self-renewal, persistence, and/or survival of stem cells and/or progenitor cells. In some embodiments, the genome-engineered iPSC-derived cells comprising one or more suicide genes further comprise one or more in/del comprised in one or more endogenous genes associated with immune response regulation and mediation, including, but not limited to, check point genes, endogenous T cell receptor genes, and MHC class I suppressor genes. In one embodiment, the genome-engineered iPSC-derived cells comprising one or more suicide genes further comprise an in/del in B2M gene, wherein the B2M is knocked out.

Additionally, applicable dedifferentiation methods and compositions for obtaining genomic-engineered hematopoietic cells of a first fate to genomic-engineered hematopoietic cells of a second fate include those depicted in, for example, International Pub. No. WO2011/159726, the disclosure of which is incorporated herein by reference. The method and composition provided therein allows partially reprogramming a starting non-pluripotent cell to a non-pluripotent intermediate cell by limiting the expression of endogenous Nanog gene during reprogramming; and subjecting the non-pluripotent intermediate cell to conditions for differentiating the intermediate cell into a desired cell type. In some embodiments, the genomically modified iPSCs and their derivative cells obtained using the methods and composition herein comprise at least one genotype listed in Table 1.

V. Therapeutic Use of Derivative Immune Cells with Functional Modalities Differentiated from Genetically Engineered iPSCs

The present invention provides, in some embodiments, a composition comprising an isolated population or subpopulation of functionally enhanced derivative immune cells that have been differentiated from genomically engineered iPSCs using the methods and compositions as disclosed herein. In some embodiments, the iPSCs of the composition comprise one or more targeted genetic edits as disclosed, which are retainable in the iPSC-derived immune cells, wherein the genetically engineered iPSCs and derivative cells therefrom are suitable for cell-based adoptive therapies. In one embodiment, the isolated population or subpopulation of genetically engineered effector cells of the composition comprises iPSC-derived CD34⁺ cells. In one embodiment, the isolated population or subpopulation of genetically engineered effector cells of the composition comprises iPSC-derived HSC cells. In one embodiment, the isolated population or subpopulation of genetically engineered effector cells of the composition comprises iPSC-derived proT or T cells. In one embodiment, the isolated population or subpopulation of genetically engineered effector cells of the composition comprises iPSC-derived proNK or NK cells. In one embodiment, the isolated population or subpopulation of genetically engineered effector cells of the composition comprises iPSC-derived immune regulatory cells or myeloid derived suppressor cells (MDSCs). In some embodiments, the iPSC-derived genetically engineered effector cells of the composition are further modulated ex vivo for improved therapeutic potential. In one embodiment of the composition, an isolated population or subpopulation of genetically engineered immune cells that have been derived from iPSCs comprises an increased number or ratio of naïve T cells, stem cell memory T cells, and/or central memory T cells. In one embodiment of the composition, the isolated population or subpopulation of genetically engineered immune cells that have been derived from iPSCs comprises an increased number or ratio of type I NKT cells. In another embodiment of the composition t, the isolated population or subpopulation of genetically engineered immune cells that have been derived from iPSCs comprises an increased number or ratio of adaptive NK cells. In some embodiments of the composition, the isolated population or subpopulation of genetically engineered CD34⁺ cells, HSC cells, T cells, NK cells, or myeloid derived suppressor cells derived from iPSCs are allogeneic. In some other embodiments of the composition, the isolated population or subpopulation of genetically engineered CD34⁺ cells, HSC cells, T cells, NK cells, or MDSC derived from iPSC are autologous.

In some embodiments of the composition, the iPSC for differentiation comprises genetic imprints selected to convey desirable therapeutic attributes in effector cells, provided that cell development biology during differentiation is not disrupted, and provided that the genetic imprints are retained and functional in the differentiated hematopoietic cells derived from said iPSC.

In some embodiments of the composition, the genetic imprints of the pluripotent stem cells comprise (i) one or more genetically modified modalities obtained through genomic insertion, deletion or substitution in the genome of the pluripotent cells during or after reprogramming a non-pluripotent cell to iPSC; or (ii) one or more retainable therapeutic attributes of a source-specific immune cell that is donor-, disease-, or treatment response-specific, and wherein the pluripotent cells are reprogrammed from the source-specific immune cell, wherein the iPSC retains the source therapeutic attributes, which are also comprised in the iPSC-derived hematopoietic lineage cells.

In some embodiments of the composition, the genetically modified modalities comprise one or more of: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells therefrom. In some embodiments of the composition, the genetically modified iPSC and the derivative cells therefrom comprise a genotype listed in Table 1. In some other embodiments of the composition, the genetically modified iPSC and the derivative cells therefrom comprising a genotype listed in Table 1 further comprise additional genetically modified modalities comprising (1) one or more of deletion or disruption of TAP1, TAP2, Tapasin, NLRC5, PD1, LAG3, TIM3, RFXANK, CIITA, RFX5, or RFXAP, RAG1, and any gene in the chromosome 6p21 region; and (2) introduction or upregulation of at least one of HLA-E, 4-1BBL, CD3, CD4, CD8, CD47, CD113, CD131, CD137, CD80, PDL1, A_{2A}R, CAR, Fc receptor, or surface triggering receptors for coupling with bi- or multi-specific or universal engagers.

In still some other embodiments of the composition, the hematopoietic lineage cells comprise the therapeutic attributes of the source-specific immune cell relating to a combination of at least two of the following: (i) one or more antigen targeting receptor expression; (ii) modified HLA; (iii) resistance to tumor microenvironment; (iv) recruitment of bystander immune cells and immune modulations; (iv) improved on-target specificity with reduced off-tumor effect; and (v) improved homing, persistence, cytotoxicity, or antigen escape rescue.

In some embodiments of the composition, the iPSC-derived hematopoietic cells comprising a genotype listed in Table 1 express at least one cytokine and/or its receptor comprising IL2, IL4, IL6, IL7, IL9, IL10, IL11, IL12, IL15, IL18, or IL21, or any modified protein thereof, and express at least a CAR. In some embodiments of the composition, the engineered expression of the cytokine(s) and the CAR(s) is NK cell specific. In some other embodiments of the composition, the engineered expression of the cytokine(s) and the CAR(s) is T cell specific. In one embodiment, the CAR comprises a CD38 binding domain. In some embodiments of the composition, the iPSC-derived hematopoietic effector cells are antigen specific. In some embodiments of the composition, the antigen specific derivative effector cells target a liquid tumor. In some embodiments of the composition, the antigen specific derivative effector cells target a solid tumor. In some embodiments of the composition, the antigen specific iPSC-derived hematopoietic effector cells are capable of rescuing tumor antigen escape.

A variety of diseases may be ameliorated by introducing the effector cells and/or compositions of the invention to a subject suitable for adoptive cell therapy, in accordance with some embodiments. In some embodiments, the iPSC-derived hematopoietic cells or compositions as provided herein are for allogeneic adoptive cell therapies. Additionally, the present invention provides, in some embodiments, therapeutic use of the above effector cells and/or therapeutic compositions and/or combination therapies by introducing the cells or composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus. Examples of hematological malignancies include, but are not limited to, acute and chronic leukemias (acute myelogenous leukemia (AML), acute lymphoblastic leukemia (ALL), chronic myelogenous leukemia (CML), lymphomas, non-Hodgkin lymphoma (NHL), Hodgkin's disease, multiple myeloma, and myelodysplastic syndromes. Examples of solid cancers include, but are not limited to, cancer of the brain, prostate, breast, lung, colon, uterus, skin, liver, bone, pancreas, ovary, testes, bladder, kidney, head, neck, stomach, cervix, rectum, larynx, and esophagus. Examples of various autoimmune disorders include, but are not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus, erythematosus, some forms of thyroiditis, some forms of uveitis, vitiligo, granulomatosis with polyangiitis (Wegener's). Examples of viral infections include, but are not limited to, HIV- (human immunodeficiency virus), HSV- (herpes simplex virus), KSHV-(Kaposi's sarcoma-associated herpesvirus), RSV- (Respiratory Syncytial Virus), EBV- (Epstein-Barr virus), CMV- (cytomegalovirus), VZV (Varicella zoster virus), adenovirus-, a lentivirus-, a BK polyomavirus-associated disorders.

The treatment using the iPSC-derived hematopoietic lineage cells of embodiments disclosed herein, or the compositions provided herein, could be carried out upon symptom presentation, or for relapse prevention. The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any intervention of a disease in a subject and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent(s) and/or compositions may be administered before, during or after the onset of a disease or an injury. The treatment of ongoing disease, where the treatment stabilizes or reduces the undesirable clinical symptoms of the patient, is also of particular interest. In particular embodiments, the subject in need of a treatment has a disease, a condition, and/or an injury that can be contained, ameliorated, and/or improved in at least one associated symptom by a cell therapy. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, a subject who has or is at risk of having a viral infection or a disease associated with a viral infection.

When evaluating responsiveness to the treatment comprising the iPSC-derived hematopoietic lineage cells of embodiments disclosed herein, the response can be measured by criteria comprising at least one of: clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST (Response Evaluation Criteria In Solid Tumors) criteria.

The therapeutic composition comprising iPSC-derived hematopoietic lineage cells as disclosed herein can be administered to a subject before, during, and/or after other treatments. As such the method of a combinational therapy can involve the administration or preparation of iPSC-derived immune cells before, during, and/or after the use of one or more additional therapeutic agents. As provided above, the one or more additional therapeutic agents comprise a peptide, a cytokine, a checkpoint inhibitor, an engager, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (JIVED). The administration of the iPSC-derived immune cells can be separated in time from the administration of an additional therapeutic agent by hours, days, or even weeks. Additionally, or alternatively, the administration can be combined with other biologically active agents or modalities such as, but not limited to, an antineoplastic agent, or a non-drug therapy, such as, surgery.

In some embodiments of a combinational cell therapy, the therapeutic combination comprises the iPSC-derived hematopoietic lineage cells provided herein and an additional therapeutic agent that is an engager, where the engager targets an antigen associated with a condition, a disease, or an indication (as described above). In some embodiments, the engager has a different tumor targeting specificity from the CAR of the engineered iPSC-derived hematopoietic lineage cells. In some embodiments, the engager is a bi-specific T cell engager (BiTE). In some embodiments, the engager is a bi-specific killer cell engager (BiKE). In some embodiments, the engager is a tri-specific killer cell engager (TriKE). In some embodiments, the engager is a multi-specific killer cell engager. In some embodiments, the engager is a universal engager compatible with multiple immune cell types.

In some embodiments of a combinational cell therapy, the therapeutic combination comprises the iPSC-derived hematopoietic lineage cells provided herein and an additional therapeutic agent that is an antibody, or an antibody fragment. In some embodiments, the antibody is a monoclonal antibody. In some embodiments, the antibody may be a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived hematopoietic lineage cells to enhance their killing ability. In some embodiments, the antibodies suitable for combinational treatment as an additional therapeutic agent to the administered with the iPSC-derived hematopoietic lineage cells include, but are not limited to, anti-CD20 (e.g., rituximab, veltuzumab, ofatumumab, ublituximab, ocaratuzumab, obinutuzumab, ibritumomab, ocrelizumab), anti-CD22 (inotuzumab, moxetumomab, epratuzumab), anti-HER2 (e.g., trastuzumab, pertuzumab), anti-CD52 (e.g., alemtuzumab), anti-EGFR (e.g., cetuximab), anti-GD2 (e.g., dinutuximab), anti-PDL1 (e.g., avelumab), anti-CD38 (e.g., daratumumab, isatuximab, MOR202), anti-CD123 (e.g., 7G3, CSL362), anti-SLAMF7 (elotuzumab), and their humanized or Fc modified variants or fragments or their functional equivalents or biosimilars. The present invention provides, in some embodiments, therapeutic compositions comprising effector cells, including the iPSC-derived hematopoietic lineage cells, having a genotype listed in Table 1 and provided herein and an additional therapeutic agent that is an antibody, or an antibody fragment, as described above.

In some embodiments, the additional therapeutic agent comprises one or more checkpoint inhibitors. Checkpoints are referred to as cell molecules, often cell surface molecules, capable of suppressing or downregulating immune responses when not inhibited. Checkpoint inhibitors are antagonists capable of reducing checkpoint gene expression or gene products, or deceasing activity of checkpoint molecules. Suitable checkpoint inhibitors for combination therapy with the derivative effector cells are provided above.

Some embodiments of the combination therapy comprising the provided derivative effector cells further comprise at least one inhibitor targeting a checkpoint molecule. Some other embodiments of the combination therapy with the provided derivative effector cells comprise two, three or more inhibitors such that two, three, or more checkpoint molecules are targeted. In some embodiments, the effector cells for combination therapy as described herein are derivative NK lineage cells as provided. In some embodiments, the effector cells for combination therapy as described herein are derivative T lineage cells. In some embodiments, the derivative NK or T lineage cells for combination therapies are functionally enhanced as provided herein. In some embodiments, the two, three or more checkpoint inhibitors may be administered in a combination therapy with, before, or after the administering of the derivative effector cells. In some embodiments, the two or more checkpoint inhibitors are administered at the same time, or one at a time (sequential). The present invention provides, in some embodiments, therapeutic compositions comprising effector cells, including the iPSC-derived effector cells, having a genotype listed in Table 1 and one or more checkpoint inhibitors, as described above.

In some embodiments, the antagonist inhibiting any of the above checkpoint molecules is an antibody. In some embodiments, the checkpoint inhibitory antibodies may be murine antibodies, human antibodies, humanized antibodies, a camel Ig, a single variable new antigen receptor (VNAR), a shark heavy-chain-only antibody (Ig NAR), chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab, aveluinab, durvalumab, ipilimumab, IPH4102, IPH43, IPH33, lirimumab, monalizumab, nivolumab, pembrolizumab, and their derivatives or functional equivalents.

The combination therapies comprising the derivative effector cells and one or more checkpoint inhibitors are applicable to treatment of liquid and solid cancers, including but not limited to cutaneous T-cell lymphoma, non-Hodgkin lymphoma (NHL), Mycosis fungoides, Pagetoid reticulosis, Sezary syndrome, Granulomatous slack skin, Lymphomatoid papulosis, Pityriasis lichenoides chronica, Pityriasis lichenoides et varioliformis acuta, CD30⁺ cutaneous T cell lymphoma, Secondary cutaneous CD30⁺ large cell lymphoma, non-mycosis fungoides CD30 cutaneous large T cell lymphoma, Pleomorphic T-cell lymphoma, Lennert lymphoma, subcutaneous T cell lymphoma, angiocentric lymphoma, blastic NK-cell lymphoma, B cell Lymphomas, hodgkins lymphoma (HL), Head and neck tumor; Squamous cell carcinoma, rhabdomyocarcoma, Lewis lung carcinoma (LLC), non-small cell lung cancer, esophageal squamous cell carcinoma, esophageal adenocarcinoma, renal cell carcinoma (RCC), colorectal cancer (CRC), acute myeloid leukemia (AML), breast cancer, gastric cancer, prostatic small cell neuroendocrine carcinoma (SCNC), liver cancer, glioblastoma, liver cancer, oral squamous cell carcinoma, pancreatic cancer, thyroid papillary cancer, intrahepatic cholangiocellular carcinoma, hepatocellular carcinoma, bone cancer, metastasis, and nasopharyngeal carcinoma.

In some embodiments, other than the derivative effector cells as provided herein, a combination for therapeutic use comprises one or more additional therapeutic agents comprising a chemotherapeutic agent or a radioactive moiety. Chemotherapeutic agent refers to cytotoxic antineoplastic agents, that is, chemical agents which preferentially kill neoplastic cells or disrupt the cell cycle of rapidly-proliferating cells, or which are found to eradicate stem cancer cells, and which are used therapeutically to prevent or reduce the growth of neoplastic cells. Chemotherapeutic agents are also sometimes referred to as antineoplastic or cytotoxic drugs or agents, and are well known in the art.

In some embodiments, the chemotherapeutic agent comprises an anthracycline, an alkylating agent, an alkyl sulfonate, an aziridine, an ethylenimine, a methylmelamine, a nitrogen mustard, a nitrosourea, an antibiotic, an antimetabolite, a folic acid analog, a purine analog, a pyrimidine analog, an enzyme, a podophyllotoxin, a platinum-containing agent, an interferon, and an interleukin. Exemplary chemotherapeutic agents include, but are not limited to, alkylating agents (cyclophosphamide, mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and gramicidine D), paclitaxel, colchicine, cytochalasin B, emetine, maytansine, and amsacrine. Additional agents include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate. Other suitable agents are those that are approved for human use, including those that will be approved, as chemotherapeutics or radiotherapeutics, and known in the art. Such agents can be referenced through any of a number of standard physicians' and oncologists' references (e.g., Goodman & Gilman's The Pharmacological Basis of Therapeutics, Ninth Edition, McGraw-Hill, N.Y., 1995) or through the National Cancer Institute website (fda.gov/cder/cancer/druglistfrarne.htm), both as updated from time to time.

Immunomodulatory drugs (IMiDs) such as thalidomide, lenalidomide, and pomalidomide stimulate both NK cells and T cells. As provided herein, IMiDs may be used with the iPSC-derived therapeutic immune cells for cancer treatments.

Other than an isolated population of iPSC-derived hematopoietic lineage cells included in the therapeutic compositions, the compositions suitable for administration to a subject/patient can further include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions of the present invention (see, e.g., Remington's Pharmaceutical Sciences, 17^{th} ed. 1985, the disclosure of which is hereby incorporated by reference in its entirety).

In one embodiment, the therapeutic composition comprises the iPSC-derived T cells made by the methods and composition disclosed herein. In one embodiment, the therapeutic composition comprises the iPSC-derived NK cells made by the methods and composition disclosed herein. In one embodiment, the therapeutic composition comprises the iPSC-derived CD34⁺ HE cells made by the methods and composition disclosed herein. In one embodiment, the therapeutic composition comprises the iPSC-derived HSCs made by the methods and composition disclosed herein. In one embodiment, the therapeutic composition comprises the iPSC-derived MDSC made by the methods and composition disclosed herein. A therapeutic composition comprising a population of iPSC-derived hematopoietic lineage cells as disclosed herein can be administered separately by intravenous, intraperitoneal, enteral, or tracheal administration methods or in combination with other suitable compounds to affect the desired treatment goals.

These pharmaceutically acceptable carriers and/or diluents can be present in amounts sufficient to maintain a pH of the therapeutic composition of between about 3 and about 10. As such, the buffering agent can be as much as about 5% on a weight to weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The invention also provides, in part, the use of a pharmaceutically acceptable cell culture medium in particular compositions and/or cultures of the present invention. Such compositions are suitable for administration to human subjects. Generally speaking, any medium that supports the maintenance, growth, and/or health of the iPSC-derived immune cells in accordance with embodiments of the invention are suitable for use as a pharmaceutical cell culture medium. In particular embodiments, the pharmaceutically acceptable cell culture medium is a serum free, and/or feeder-free medium. In various embodiments, the serum-free medium is animal-free, and can optionally be protein-free. Optionally, the medium can contain biopharmaceutically acceptable recombinant proteins. “Animal-free medium” refers to a medium wherein the components are derived from non-animal sources. Recombinant proteins replace native animal proteins in animal-free medium and the nutrients are obtained from synthetic, plant or microbial sources. “Protein-free medium,” in contrast, is defined as substantially free of proteins. One having ordinary skill in the art would appreciate that the above examples of media are illustrative and in no way limit the formulation of media suitable for use in the present invention and that there are many suitable media known and available to those in the art.

The isolated iPSC-derived hematopoietic lineage cells can have at least 50%, 60%, 70%, 80%, 90%, 95%, 98%, or 99% T cells, NK cells, NKT cells, proT cells, proNK cells, CD34⁺ HE cells, HSCs, B cells, myeloid-derived suppressor cells (MDSCs), regulatory macrophages, regulatory dendritic cells, or mesenchymal stromal cells. In some embodiments, the isolated iPSC-derived hematopoietic lineage cells comprise about 95% to about 100% T cells, NK cells, proT cells, proNK cells, CD34⁺ HE cells, or myeloid-derived suppressor cells (MDSCs). In some embodiments, the present invention provides therapeutic compositions having purified T cells or NK cells, such as a composition having an isolated population of about 95% T cells, NK cells, proT cells, proNK cells, CD34⁺ HE cells, or myeloid-derived suppressor cells (MDSCs) to treat a subject in need of the cell therapy.

In various embodiments, the combinational cell therapy, or composition used therefor, comprises a therapeutic protein or peptide that is a CD3 engager and a population of NK cells derived from genomically engineered iPSCs comprising a genotype listed in Table 1, wherein the derived NK cells comprise an exogenous TCR complex (TCR^{exo}) that recognizes a first tumor antigen. In one embodiment of the combinational cell therapy, or composition used therefor, the derived NK cells further comprise a chimeric antigen receptor (CAR) or an engager targeting at least a second tumor antigen, and optionally an exogenous CD16 or variant thereof and/or one or more exogenous cytokines. In certain embodiments, the CAR targets CD19, BCMA, MICA/B, MR1, CD38, CD20, CD22, or CD123. In another embodiment, the combinational cell therapy, or composition used therefor, comprises a therapeutic protein or peptide that is a CD3 engager and a population of T cells derived from genomically engineered iPSCs comprising a genotype listed in Table 1, wherein the derived T cells comprise an exogenous TCR complex (TCR^{exo}) that recognizes a first tumor antigen. In yet another embodiment, the derived T cells further comprise a chimeric antigen receptor (CAR) or an engager targeting at least a second tumor antigen, and optionally an exogenous CD16 or variant thereof and/or one or more exogenous cytokines. In certain embodiments, the CAR targets CD19, BCMA, MICA/B, MR1, CD38, CD20, CD22, or CD123. In some embodiments, the combinational cell therapy comprises one of blinatumomab, catumaxomab, ertumaxomab, R06958688, AFM11, MT110/AMG 110, MT111/AMG211/MEDI-565, AMG330, MT112/BAY2010112, MOR209/ES414, MGD006/S80880, MGD007, and/or FBTA05, and a population of NK or T cells derived from genomically engineered iPSCs comprising a genotype listed in Table 1. In yet some other embodiments, the combinational cell therapy, or composition used therefor, comprises one of blinatumomab, catumaxomab, and ertumaxomab, and a population of NK or T cells derived from genomically engineered iPSCs comprising a genotype listed in Table 1. In still some additional embodiments, the combinational cell therapy, or composition used therefor, comprises one of blinatumomab, catumaxomab, and ertumaxomab, and a population of NK or T cells derived from genomically engineered iPSCs comprising a genotype listed in Table 1, wherein the derived effector cells comprise a TCR^{exo}, optionally a CAR, and optionally one or more of an engager a CFR, exogenous CD16 or a variant thereof, CD38 knock out and/or one or more exogenous cytokines.

As a person of ordinary skill in the art would understand, both autologous and allogeneic hematopoietic lineage cells derived from iPSC based on the methods and compositions provided herein can be used in cell therapies as described above. For autologous transplantation, the isolated population of derived hematopoietic lineage cells are either complete or partial HLA-match with the patient. In another embodiment, the derived hematopoietic lineage cells are not HLA-matched to the subject, wherein the derived hematopoietic lineage cells are NK cells or T cell with HLA-I and/or HLA-II deficiency.

In some embodiments, the number of derived hematopoietic lineage cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, or at least 5×10⁹ cells, per dose. In some embodiments, the number of derived hematopoietic lineage cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose; about 3×10⁹ cells to about 3×10¹⁰ cells, per dose, or any range in-between. Generally, 1×10⁸ cells/dose translates to 1.67×10⁶ cells/kg for a 60 kg patient.

In one embodiment, the number of derived hematopoietic lineage cells in the therapeutic composition is the number of immune cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×6 cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of derived hematopoietic lineage cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of cells provided to a subject is from about 2×10⁶ cells/kg to about 10×10⁶ cells/kg, about 3×10⁶ cells/kg to about ×10⁶ cells/kg, about 4×10⁶ cells/kg to about 10×10⁶ cells/kg, about 5×10⁶ cells/kg to about ×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×6 cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×6 cells/kg to about 8×10⁶ cells/kg, or 6×10⁶ cells/kg to about 8×10⁶ cells/kg, including all intervening doses of cells.

In some embodiments, the therapeutic use of derived hematopoietic lineage cells is a single-dose treatment. In some embodiments, the therapeutic use of derived hematopoietic lineage cells is a multi-dose treatment. In some embodiments, the multi-dose treatment is one dose every day, every 3 days, every 7 days, every 10 days, every 15 days, every 20 days, every days, every 30 days, every 35 days, every 40 days, every 45 days, or every 50 days, or any number of days in-between. In some embodiments, the multi-dose treatment comprises three, or four, or five, once-weekly doses. In some embodiments of the multi-dose treatment comprising three, or four, or five, once-weekly doses, the treatment further comprises an observation period for determining whether additional single or multi doses are needed.

The compositions comprising a population of derived hematopoietic lineage cells of the invention can be sterile, and can be suitable and ready for administration (i.e., can be administered without any further processing) to human patients. A cell-based composition that is ready for administration means that the composition does not require any further processing or manipulation prior to transplant or administration to a subject. In other embodiments, the invention provides an isolated population of derived hematopoietic lineage cells that are expanded and/or modulated prior to administration with one or more agents. For derived hematopoietic lineage cells that are genetically engineered to express recombinant TCR or CAR, the cells can be activated and expanded using methods as described, for example, in U.S. Pat. No. 6,352,694, the entire content of which is incorporated herein by reference.

In certain embodiments, the primary stimulatory signal and the co-stimulatory signal for the derived hematopoietic lineage cells can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent which will bind to the agents such as disclosed in U.S. Pub. Nos. 2004/0101519 and 2006/0034810 for artificial antigen presenting cells (aAPCs) that are contemplated for use in activating and expanding T lymphocytes in embodiments of the present invention.

Some variation in dosage, frequency, and protocol will necessarily occur depending on the condition of the subject being treated. The person responsible for administration will, in any event, determine the appropriate dose, frequency and protocol for the individual subject.

### EXAMPLES

The following examples are offered by way of illustration and not by way of limitation.

### Example 1—Materials and Methods

To effectively select and test suicide systems under the control of various promoters in combination with different safe harbor loci integration strategies, a proprietary hiPSC platform of the applicant was used, which enables single cell passaging and high-throughput, 96-well plate-based flow cytometry sorting, to allow for the derivation of clonal hiPSCs with single or multiple genetic modulations.

hiPSC Maintenance in Small Molecule Culture: hiPSCs were routinely passaged as single cells once confluency of the culture reached 75%-90%. For single-cell dissociation, hiPSCs were washed once with PBS (Mediatech) and treated with Accutase (Millipore) for 3-5 min at 37° C. followed with pipetting to ensure single-cell dissociation. The single-cell suspension was then mixed in equal volume with conventional medium, centrifuged at 225×g for 4 min, resuspended in FMM, and plated on Matrigel-coated surface. Passages were typically 1:6-1:8, transferred tissue culture plates previously coated with Matrigel for 2-4 hr in 37° C. and fed every 2-3 days with FMM. Cell cultures were maintained in a humidified incubator set at 37° C. and 5% CO₂.

Human iPSC engineering with ZFN, CRISPR for targeted editing of modalities of interest: Using ROSA26 targeted insertion as an example, for ZFN mediated genome editing, 2 million iPSCs were transfected with a mixture of 2.5 μg ZFN-L (FTV893), 2.5 μg ZFN-R (FTV894) and 5 μg donor construct, for AAVS1 targeted insertion. For CRISPR mediated genome editing, 2 million iPSCs were transfected with a mixture of 5 μg ROSA26-gRNA/Cas9 (FTV922) and 5 μg donor construct, for ROSA26 targeted insertion. Transfection was done using Neon transfection system (Life Technologies) using parameters 1500V, 10 ms, 3 pulses. On day 2 or 3 after transfection, transfection efficiency was measured using flow cytometry if the plasmids contain artificial promoter-driver GFP and/or RFP expression cassette. On day 4 after transfection, puromycin was added to the medium at concentration of 0.1 μg/ml for the first 7 days and 0.2 μg/ml after 7 days to select the targeted cells. During the puromycin selection, the cells were passaged onto fresh matrigel-coated wells on day 10. On day 16 or later of puromycin selection, the surviving cells were analyzed by flow cytometry for GFP⁺ iPS cell percentage.

Bulk sort and clonal sort of genome-edited iPSCs: iPSCs with genomic targeted editing using ZFN or CRISPR-Cas9 were bulk sorted and clonal sorted for GFP⁺SSEA4⁺TRA181⁺ iPSCs after 20 days of puromycin selection. Single cell dissociated targeted iPSC pools were resuspended in chilled staining buffer containing Hanks' Balanced Salt Solution (MediaTech), 4% fetal bovine serum (Invitrogen), 1× penicillin/streptomycin (Mediatech) and 10 mM Hepes (Mediatech); made fresh for optimal performance. Conjugated primary antibodies, including SSEA4-PE, TRA181-Alexa Fluor-647 (BD Biosciences), were added to the cell solution and incubated on ice for 15 minutes. All antibodies were used at 7 μL in 100 μL staining buffer per million cells. The solution was washed once in staining buffer, spun down at 225 g for 4 minutes and resuspended in staining buffer containing 10 μM Thiazovivn and maintained on ice for flow cytometry sorting. Flow cytometry sorting was performed on FACS Aria II (BD Biosciences). For bulk sort, GFP⁺SSEA4⁺TRA181⁺ cells were gated and sorted into 15 ml canonical tubes filled with 7 ml FMM. For clonal sort, the sorted cells were directly ejected into 96-well plates using the 100 μM nozzle, at concentrations of 3 events per well. Each well was prefilled with 200 μL FMM supplemented with 5 μg/mL fibronectin and 1×penicillin/streptomycin (Mediatech) and previously coated overnight with 5×Matrigel. 5×Matrigel precoating includes adding one aliquot of Matrigel into 5 mL of DMEM/F12, then incubating overnight at 4° C. to allow for proper resuspension and finally adding to 96-well plates at 50 μL per well, followed by overnight incubation at 37° C. The 5×Matrigel is aspirated immediately before the addition of media to each well. Upon completion of the sort, 96-well plates were centrifuged for 1-2 min at 225 g prior to incubation. The plates were left undisturbed for seven days. On the seventh day, 150 μL of medium was removed from each well and replaced with 100 μL FMM. Wells were refed with an additional 100 μL FMM on day 10 post sort. Colony formation was detected as early as day 2 and most colonies were expanded between days 7-10 post sort. In the first passage, wells were washed with PBS and dissociated with 30 μL Accutase for approximately 10 min at 37° C. The need for extended Accutase treatment reflects the compactness of colonies that have sat idle in culture for prolonged duration. After cells are seen to be dissociating, 200 μL of FMM is added to each well and pipetted several times to break up the colony. The dissociated colony is transferred to another well of a 96-well plate previously coated with 5×Matrigel and then centrifuged for 2 min at 225 g prior to incubation. This 1:1 passage is conducted to spread out the early colony prior to expansion. Subsequent passages were done routinely with Accutase treatment for 3-5 min and expansion of 1:4-1:8 upon 75-90% confluency into larger wells previously coated with 1×Matrigel in FMM. Each clonal cell line was analyzed for GFP fluorescence level and TRA1-81 expression level. Clonal lines with near 100% GFP⁺ and TRA1-81⁺ were selected for further PCR screening and analysis, and cryopreserved as a master cell bank. Flow cytometry analysis was performed on Guava EasyCyte 8 HT (Millipore) and analyzed using Flowjo (FlowJo, LLC).

### Example 2— Expression of MR1-TCR in TiPSCs and FiPSCs

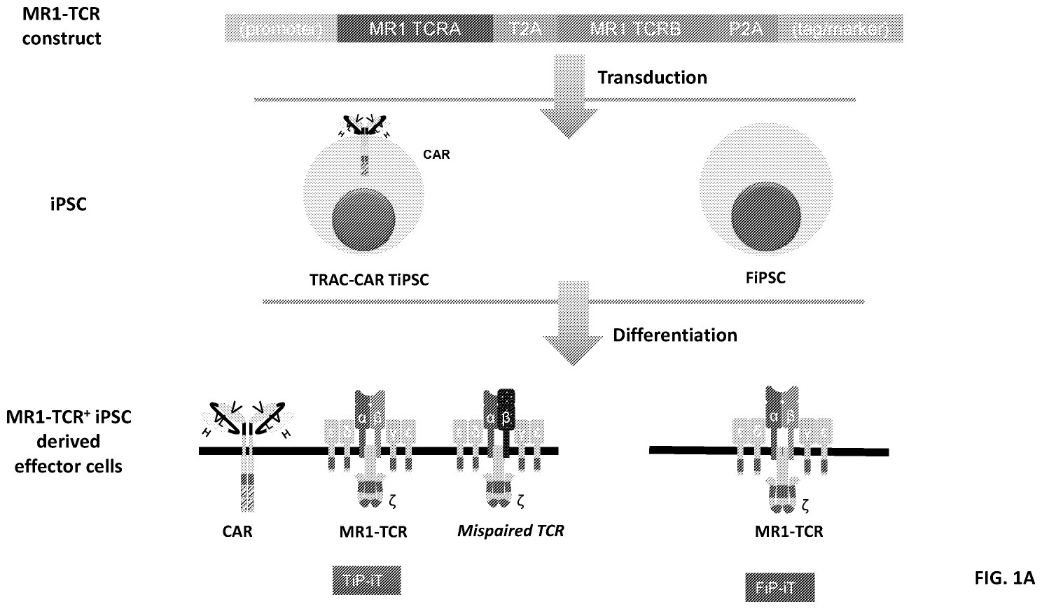

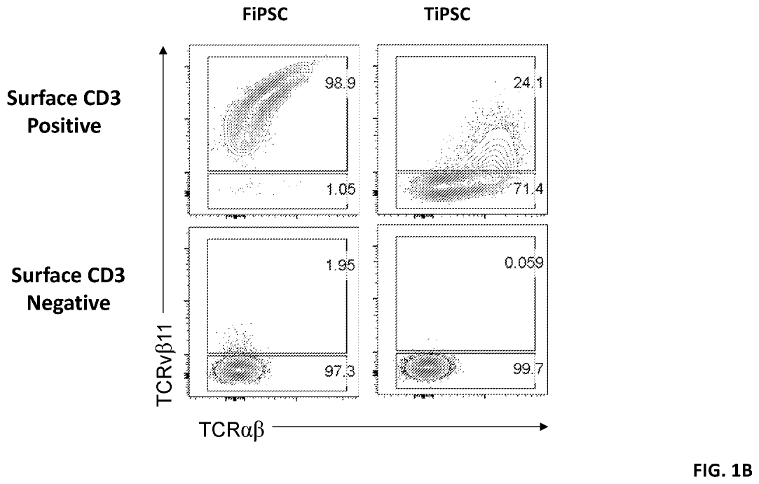

The non-polymorphic MHC class I-related protein, MR1, is widely expressed at the cell surface of cancer cells with minimal variability among patients and enables the unique prospect to be a universal cancer immunotherapeutic target. As illustrated in FIG. 1A, an MR1 clonal T cell receptor (TCR) construct was transduced into iPSC reprogrammed from T cells (TiPSC) containing a CAR at the TRAC locus (CAR/TCR⁻ TiPSC) or into iPSC reprogrammed from fibroblasts (FiPSC). T cells (TiPSC) containing a CAR at the TRAC locus is also referred to as “TRAC-CAR TiPSC” from time to time in this application. The benefits of knocking out the endogenous TCR complex in an effector T lineage cell by disrupting either TCRα or TCRβ chain expression includes the avoidance of GvHD in adoptive cell therapy. When differentiated into iT cells, the MR1-TCR/CAR/TCR⁻ T lineage effector cells (TiP-iT) express a CAR and an MR1-TCR. It is also discovered that the endogenous TCRβ chain still expressed in TiP-iT and could form a mispaired TCR with the exogenous TCRα chain provided by the MR1-TCR construct, which affects the amount of MR1-TCR that is available for its antigen specific tumor targeting.

Unlike TiP-iT, FiPSC-derived T lineage cells (FiP-iT) do not have a pre-rearranged V(D)J segment of TCRα and TCRβ, which, without being bound by theory, may be why it takes much longer for FiPSC-derived iT cells to mature and to express endogenous TCR. The delayed endogenous TCR expression leads to the observation that there is no TCR mispairing issue for FiP-iT as compared to TiP-iT.

To evaluate the extent of TCR mispairing in TiP-iT cells, surface staining was performed and analyzed by a BD LSRFortessa™ flow cytometer. MR1-TCR transduced FiPSC and TiPSC cells from D35 differentiation were stained with TCRvβ11 (an MR1-TCRβ chain marker), TCRαβ, CD3 and CD45 for analysis. The bottom panel of the upper right quadrant of FIG. 1B shows the portion of mispaired TCR complexes in TiPSC-iTs (CD3 positive) that are positive in TCR staining but low on TCRvβ11 (TCRvβ11⁻ TCRαβ⁺), indicating the mispaired nature of the TCR complex in this portion, and that the endogenous TCRβ in TiPSC leads to TCR mispairing in TiP-iT.

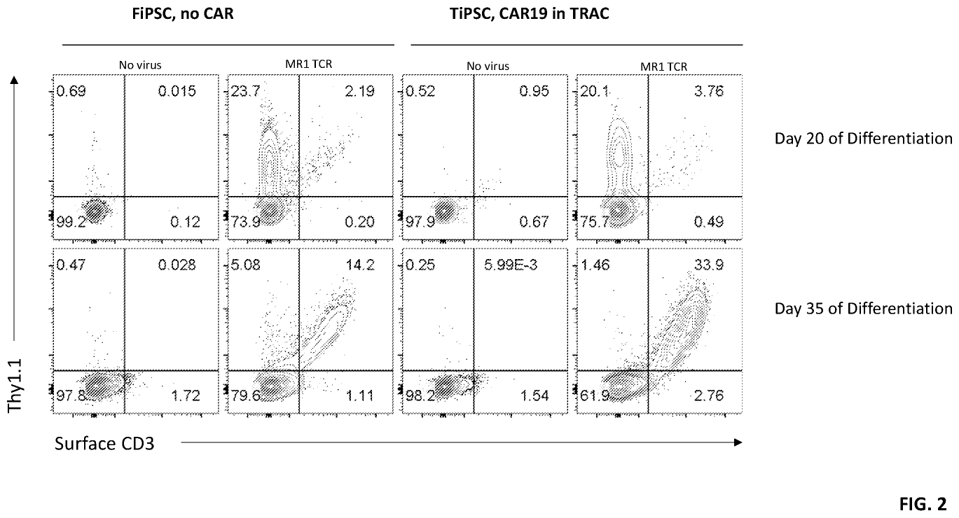

Further, the cells were stained on around day 20 or 35 of the FiPSC or TRAC-CAR TiPSC T lineage differentiation process for the presence of Thy1.1, and for CD3 present on the surface of the derivative cells. As shown in FIG. 2, Thy1.1 positive cells, which should be expressing TCRαβ, allow retention of endogenous CD3 molecules on the cell surface. However, in the absence of MR1-TCR transduction, surface CD3 expression is not observed because FiPSCs do not express either TCRα or TCRβ chains, whereas TRAC-CAR TiPSCs do not express TCRα chains to form the TCR complex necessary for CD3 cell surface presentation. Therefore, MR1-TCR, as shown, can stabilize surface expression of CD3 molecules in effector cells that are negative in endogenous TCR expression, including T cells with TCRα knockout and FiPSC-derived T lineage cells.

### Example 3— Functional Characterization of Effector Cells Expressing MR1-TCR

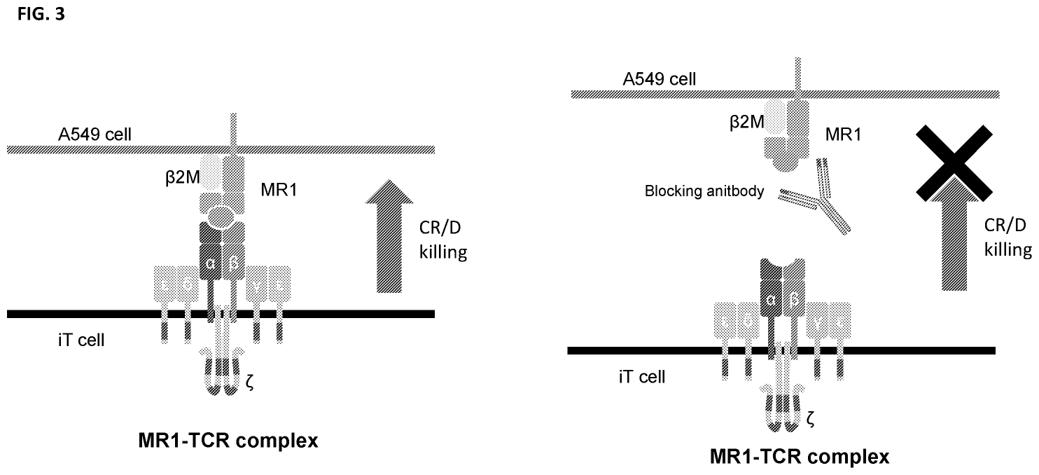

To assess MR1-dependent cytokine release and degranulation (CR/D) response, which is indicative of activated effector cells with the ability of targeted killing, MR1-TCR transduced FiP-iT and TiP-iT cells from D42 differentiation were co-cultivated with a 50:50 mixture of A549 lung carcinoma cells, which are known to present tumor-associated metabolites with the MR1 molecule. The cells were co-cultured in the absence (A549) or presence (A549+Ab) of an MR1 blocking antibody at 10 μg/ml. Plates were incubated at around 37° C. and about 5% CO₂ in a humidified incubator for 5 hours, followed by live/dead staining, surface staining, and intracellular staining that were analyzed by a BD LSRFortessa™ flow cytometer to test whether the MR1 blocking antibody would prevent the MR1-dependent CR/D cell response, as illustrated in FIG. 3.

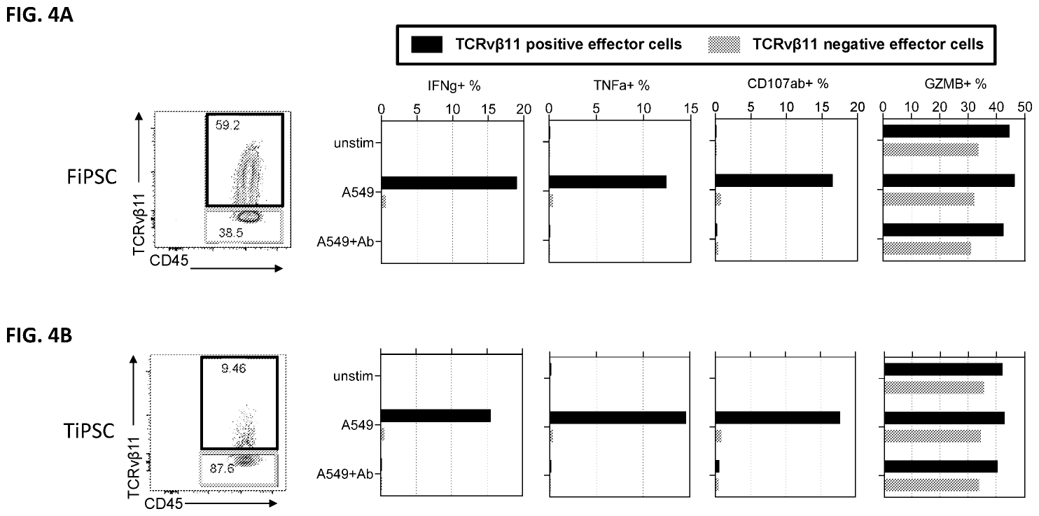

MR1-TCR dependent response was analyzed by comparing TCRvβ11 positive and negative populations, with effector cells without the addition of A549 being used as an unstimulated control (unstim). As shown in FIGS. 4A and 4B, representative FACS plots demonstrate the gating for MR1-TCR⁺ (black) and MR1-TCR⁻ (grey) cells, and the bar graphs summarize cytokine production for IFNγ, TNFα, CD107ab and granzyme B in MR1-TCR^{+/-} cells derived from FiPSC (FIG. 4A) or TiPSC (FIG. 4B). It is shown that the cytokine production for IFNγ, TNFα, CD107ab is MR1-TCR dependent, and the production of granzyme B is enhanced by MR1-TCR.

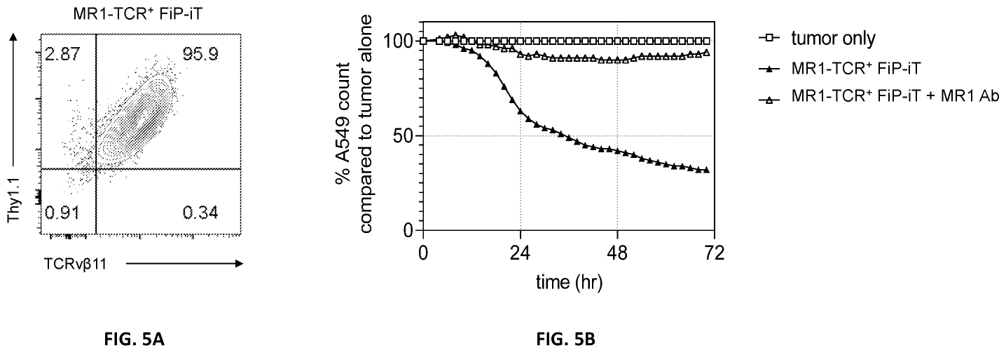

Cytotoxicity of the effector cells was assessed by co-cultivating magnetically enriched MR1-TCR expressing FiP-iT from D42 differentiation (effector) with A549 (target) at about a 3:1 effector to target (E:T) ratio in the absence (MR1-TCR⁺ FiP-iT) or presence (MR1-TCR⁺ FiP-iT+MR1 Ab) of 10 μg/ml MR1 blocking antibody. The well containing tumor alone without the effector cells was included as control. A549-NLR counts were monitored by IncuCyte™ for 72 hours after the addition of effector cells. The percentage of A549 count was normalized to tumor alone and plotted from 0 hr to 72 hr. As shown in FIG. 5A, a representative FACS plot demonstrates the purity of Thy1.1⁺ TCRvβ11⁺ cells, representing FiP-iT cells expressing MR1-TCR, within the input population. FIG. 5B demonstrates the in vitro 72-hr MR1-TCR dependent killing capacity of MR1-TCR effector cells against A549 lung cancer cells.

### Example 4— Effector Cells Expressing MR1-TCR are Compatible with BiTE Targeting

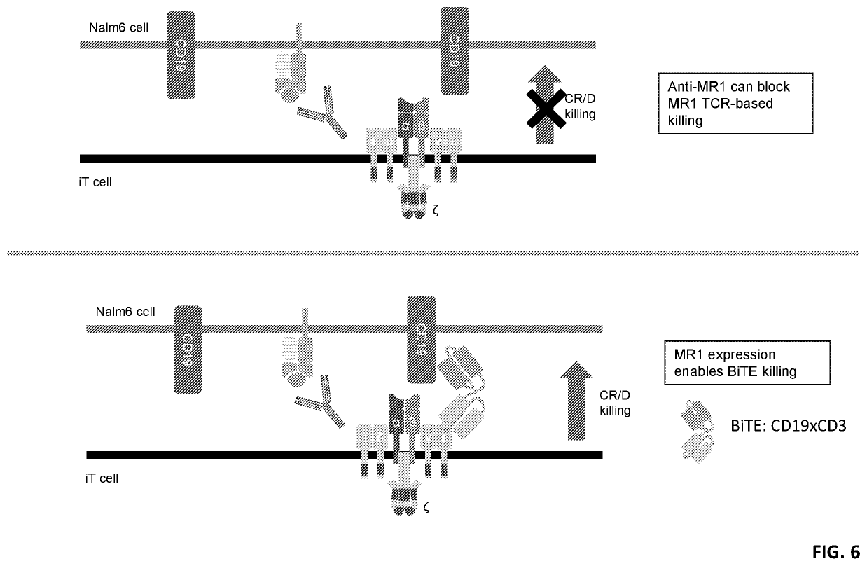

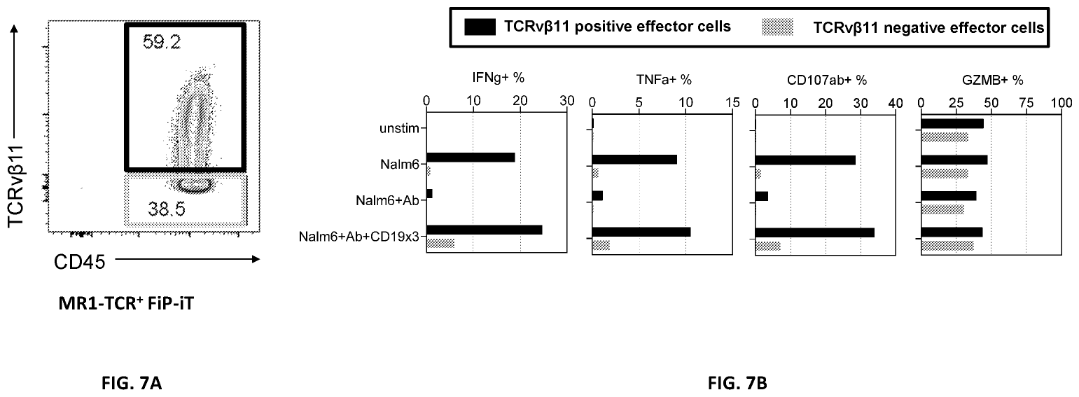

To test if the MR1-TCR expressing effector cells are compatible with BiTE targeting, a CD3-based BiTE is used for the proof of concept assessment, with the notion that MR1-TCR enables CD3 cell surface presentation (cs-CD3; see CD3ε, δ, γ subunits in the MR1-TCR complex in FIG. 6) in both TiP-iT with endogenous TCR knockout and FiP-iT which do not have endogenous TCR, therefore the cs-CD3 is necessary for a CD3-based BiTE to engage the effector cells for targeted killing. CR/D was assessed by co-cultivating MR1-TCR transduced FiPSC cells from D42 differentiation with a 50:50 mixture of Nalm6 cells in the absence (Nalm6) or presence (Nalm6⁺Ab) of MR1 blocking antibody at 10 μg/ml. For BiTE experiments, an anti-CD19×CD3 BiTE (Invivogen, San Diego, CA) was tested with the Nalm6+Ab group to trigger CD19-specific TCR signaling in the absence of MR1 antigen (Nalm6+Ab+CD19×3). Effector cells without the addition of Nalm6 were used as an unstimulated control (unstim). Plates were incubated at around 37° C. and about 5% CO₂ in a humidified incubator for 5 hours, followed by live/dead staining, surface staining, and intracellular staining, and analyzed by a BD LSRFortessa™ flow cytometer. MR1-TCR dependent response was analyzed by comparing TCRvβ11 positive and negative populations. As shown in FIG. 7A, a representative FACS plot demonstrates the gating for MR1-TCR⁺ cells and MR1-TCR⁻ cells, whereas the bar graphs in FIG. 7B summarize cytokine production of IFNγ, TNFα, CD107ab and granzyme B by MR1-TCR⁺ (black) and MR1-TCR⁻ (grey) cells in response to conditions such as: no stimulation (unstim), Nalm6 only, Nalm with MR1 blocking antibody (Nalm6+Ab), and Nalm6 with MR1 blocking antibody in the presence of the BiTE (Nalm6+Ab+CD19×3). As shown, when the MR1 blocking antibody presented the MR1-TCR mediated cell responses, the BiTE through binding of the cs-CD3 of the effector cell leads to BiTE-specific cell killing responses including, but not limited to cytokine release and production of granzyme B.

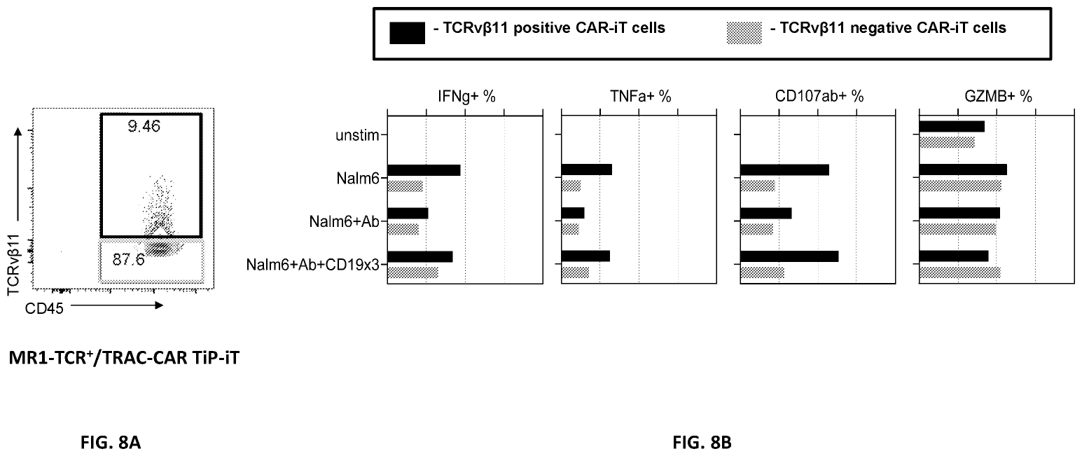

CR/D was also assessed by co-cultivating MR1-TCR transduced TiPSC cells from D42 differentiation with a 50:50 mixture of Nalm6 cells in the absence (Nalm6) or presence (Nalm6+Ab) of MR1 blocking antibody at 10 μg/ml. The bar graphs in FIG. 8B summarize cytokine production for IFNγ, TNFα, CD107ab and granzyme B for MR1-TCR⁺ (black) and MR1-TCR⁻ (grey) cells (as gated in FIG. 8A) in response to no stimulation (unstim), Nalm6, Nalm with MR1 blocking antibody (Nalm6+Ab), and Nalm6 with MR1 blocking antibody in the presence of a CD19×CD3 BiTE (Nalm6+Ab+CD19×3). Grey bars demonstrate the basal CR/D level for MR1-TCR transduced TiPSC attributed to CD19-CAR triggered response. The increased CR/D level shown by the black bars (MR1-TCR⁺ TiPSC) in comparison to grey bars (MR1-TCR⁻ TiPSC) defines MR1-TCR mediated response in the presence of a CAR stimulation.

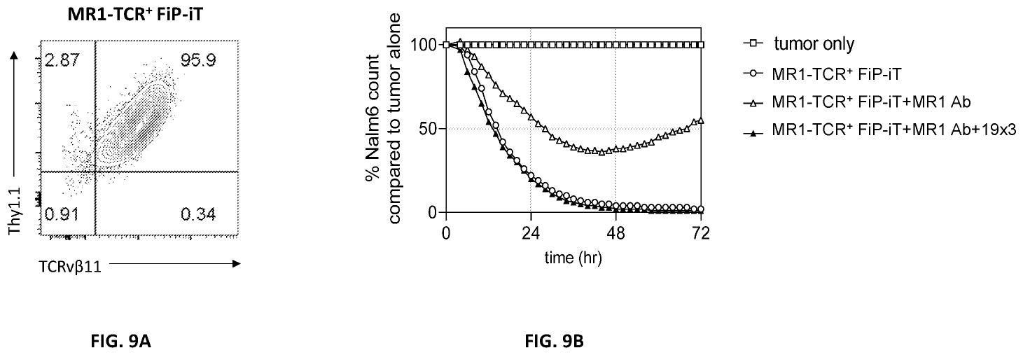

Further, to demonstrate that the addition of a BiTE restores cytotoxicity in an MR1-dependent manner, magnetically enriched MR1-TCR expressing FiPSC-derived effector cells from D42 differentiation were co-cultivated with Nalm6-NLR (target) at a 1:1 E:T ratio in the absence (MR1-TCR⁺ FiP-iT) or presence (MR1-TCR⁺ FiP-iT+MR1 Ab) of 10 μg/ml MR1 blocking antibody. CD19×3 BiTE was tested with the MR1-TCR⁺ FiP-iT in the presence of MR1 blocking antibody to trigger CD19-specific TCR signaling in the absence of MR1 antigen (MR1-TCR⁺ FiP-iT+MR1 Ab+19×3). A well containing tumor alone was included as control (tumor only). Nalm6-NLR counts were monitored by IncuCyte for 72 hours after the addition of effectors. The percentage of Nalm6 count was normalized to tumor alone and plotted from 0 hr to 72 hr. As shown in FIG. 9A, a representative FACS plot demonstrates the purity of Thy1.1⁺ TCRvβ11⁺ cells (MR1-TCR⁺ FiP-iT) within the input population. The graph in FIG. 9B demonstrates the in vitro 72-hr killing capacity of MR1-TCR⁺ FiP-iT cells against Nalm6 in the absence/presence of MR1 blocking antibody and BiTE CD19×CD3. The BiTE targeted killing through CD19 recognition rescued the loss of MR1-TCR directed cytotoxicity in the presence of MR1 blocking antibody, and restored the MR1-mediated TCR signaling.

As such, the addition of BiTEs allows targeting of a tumor cell by engaging the effector cell via a surface triggering protein including, but not limited to, CD3, even when the antigen targeted by an exogenous TCR or CAR is blocked, thereby providing proof of concept for the ability of an effector cell to overcome tumor antigen escape by utilizing any combination between a BiTE, a TCR and a CAR, which collectively have two or more targeting specificities.

### Example 5— Further TRBC Knockout in TiPSC Corrects Mispairing and Improves Exogenous TCR Function

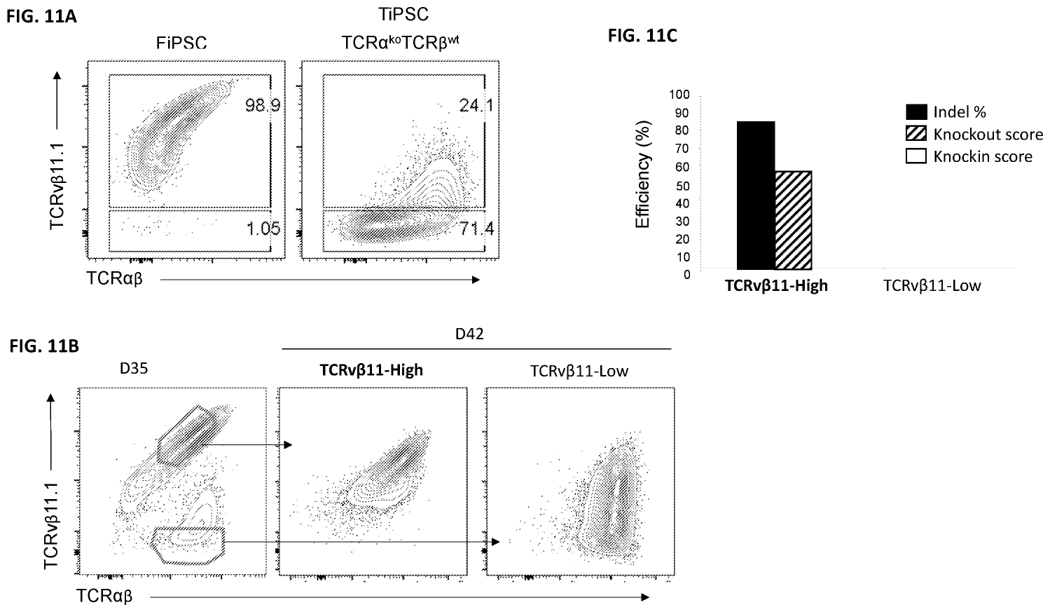

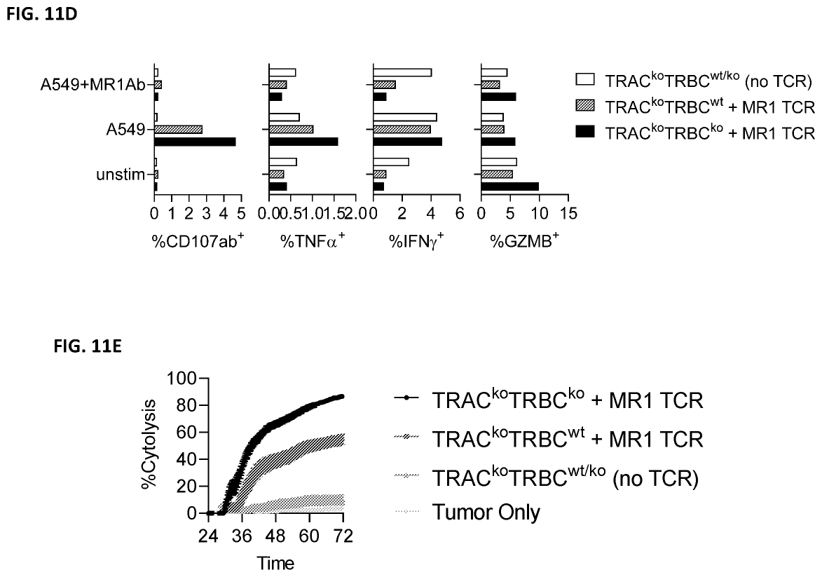

Surface staining was performed to evaluate TCR mispairing. MR1-TCR transduced FiPSCs and TiPSCs (TCRα^{ko}TCRβ^{wt}) from D35 differentiation were first stained with TCRvβ11, then stained with TCRαβ, CD3 and CD45 and analyzed by LSRFortessa™. As shown in FIG. 11A, representative FACS plots demonstrate the TCRvβ11 and TCRαβ expression of surface CD3⁺ cells derived from FiPSCs and TiPSCs, indicating that the mispairing issue is present in TiPSC with only TRAC knockout, but not in FiPSC. D35 TiPSC having both TRAC and TRBC knockout were stained with TCRvβ11 and TCRαβ, Thy1.1 and CD45 and analyzed by LSRFortessa™. As presented in FIG. 11B, a representative FACS plot demonstrates the TCRvβ11 and TCRαβ expression, showing that the mispairing is reduced by knocking out both TCRα and TCRβ, for example, by disrupting TRAC and TRBC in TiPSC cells. Sequencing analysis of a sorted TCRvβ11.1^{+/-} population confirmed efficient TRBC KO in the TCRvβ11.1⁺ cells, while TRBC is still intact in the TCRvβ11.1⁻ population (FIG. 11C).

Further, the degree of TCR complex mispairing was shown to negatively correlate with TCR function, such as cytokine secretion and tumor cell lysis. Cytokine release and degranulation was assessed by co-cultivating indicated iT cells with A549 cells at 1 to 1 ratio, in the absence (A549) or presence (A549⁺Ab) of MR1 blocking antibody at about 10 μg/ml. Plates were incubated overnight, followed by live/dead staining, surface staining, intracellular staining and analysis by a BD LSRFortessa™ flow cytometer. Effector cells without the addition of A549 were used as an unstimulated control (unstim). Cytokine production for CD107ab, TNFα, IFNγ and granzyme B for indicated iT cells is shown in FIG. 11D.

TCR-mediated cytotoxicity was assessed by co-cultivating indicated iT cells with A549 cells at a 1 to 1 ratio. The tumor alone and iT cells without MR1-TCR were included as controls (tumor only and TRAC^{ko}TRBC^{wt/ko} no TCR). A549 cells were monitored by xCELLigence™ for 72 hr after addition of the effector cells. Cytolysis was normalized to tumor alone and plotted from 0 hr to 72 hr. FIG. 11E demonstrates the in vitro killing capacity of MR1-TCR⁺ transduced iT cells against A549 cells with or without TRBC KO. As shown, the impairment of the TCR function including tumor cell killing was corrected by further knocking out TCRβ in the cell expressing the exogenous TCR. NYESO1-TCR was tested using the same assay with similar correlation observed.

### Example 6— Stepwise Engineering of iPSC and Validation of Modified Derivative Effector Cells

To combine the potent targeted therapy of a chimeric antigen receptor (CAR) with universal targeting of secondary and tertiary antigens, an exemplary MR1 clonal T cell receptor (MR1-TCR) and a high-affinity non-cleavable CD16 Fc receptor (hnCD16) were expressed in iPSC-derived CAR19 T cells (CAR19-iT cells) or iPSC-derived CAR-BCMA T cells (CAR-BCMA iT cells) directed to leukemia and lymphoma, and in CAR-MICAS T cells directed to solid tumors. As demonstrated above, the MR1-TCR allows highly specific recognition of tumor associated antigen presented by the MR1 protein. The non-polymorphic MHC class I-related protein MR1 is widely expressed with minimal variability among patients and enables the unique prospect to be a universal cancer immunotherapy by using the cognate MR1-TCR. The hnCD16 Fc receptor has been shown to improve antibody-dependent cellular cytotoxicity (ADCC) leveraging the broad range of available therapeutic monoclonal antibodies to target clinically validated tumor antigens.

A preliminary assessment demonstrated that MR1-TCR overexpressed in T cells allowed for enhanced recognition of multiple hematological and solid tumor cell lines. Notably, prominent target specific killing was seen in A549 lung carcinoma cells (>75% reduction in total viable cells) with the directed cytotoxicity specifically inhibited by an MR1 blocking antibody. Next, in vitro functional testing was performed on the engineered CAR19-iT cells in co-culture assays where killing of tumor cells via MR1-TCR engagement and via hnCD16-mediated ADCC was measured. Specifically, it was observed that CAR19-iT cells expressing hnCD16 can be efficiently directed to lyse CD20⁺ Raji cells in the presence of rituximab or HER2⁺ SKOV3 cells in the presence of Herceptin, demonstrating the potential to target both hematological malignancies and solid tumors with one target modality in combination with various monoclonal antibodies. Moreover, CAR19-iT cells expressing either MR1-TCR or hnCD16 showed the ability to control growth of CD19 KO lymphoma cells in the co-culture assays, further highlighting the unique ability to elicit multiple ways to target antigen escape. In summary, the advances presented here demonstrate that both the MR1-TCR and hnCD16 modalities synergize with CAR-iT cells as an off-the-shelf therapeutic that can provide durable responses and enable broad applicability for targeting of additional tumor antigens where single-agent therapeutics fail to provide clinical benefit for patients.

In addition, MR1-TCR is inserted at TRBC in a TRAC-CAR TiPSC to knockout endogenous TCRβ, thereby reducing or eliminating a mispaired TCR complex consisting of an MR1-TCRα and an endogenous TCRβ, and maximizing the level of the exogenous MR1-TCR for targeted killing. The resultant derivative T lineage effector cells are tested using the same assays to confirm their improved cytotoxicity in an MR1-dependent manner, and their blocked MR1-mediated TCR signaling in the presence of a MR1 blocking antibody can also be restored by the addition of a CD3-based BiTE.

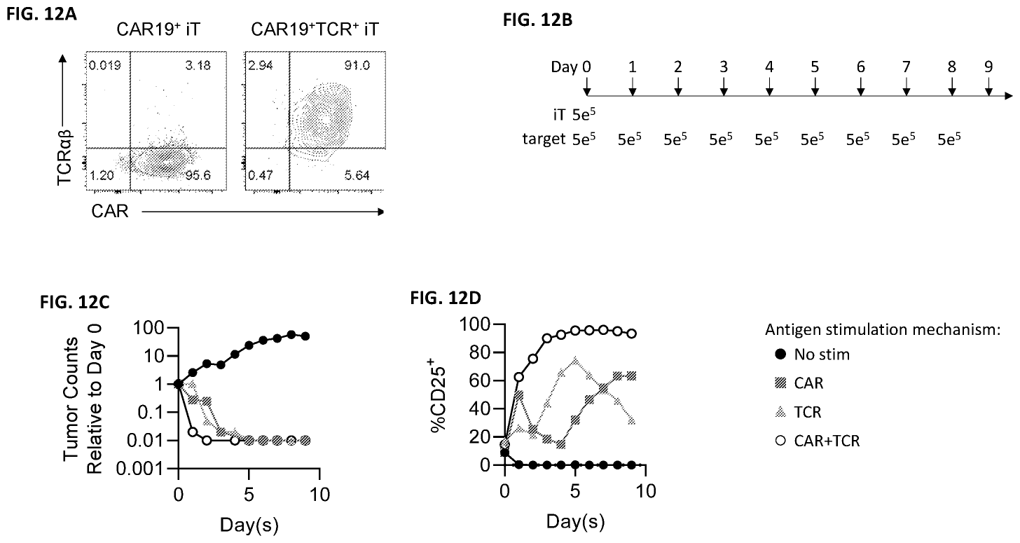

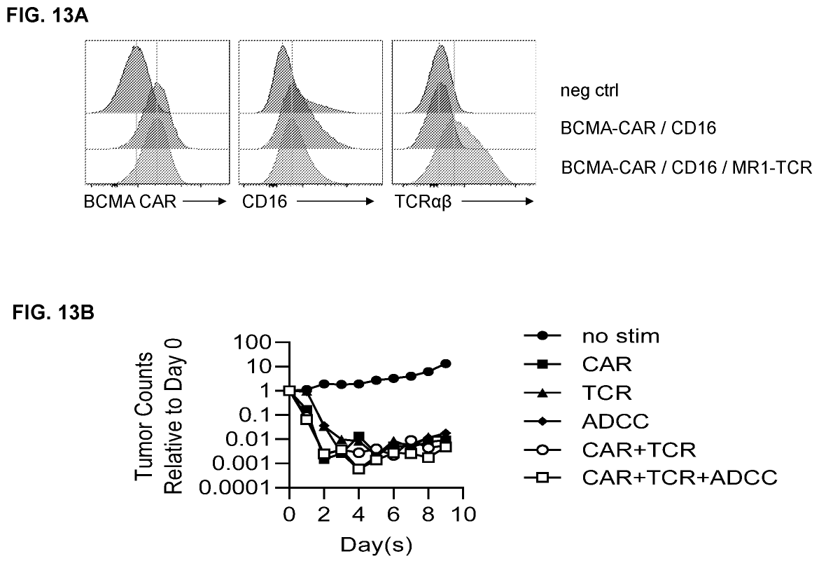

To evaluate the compatibility of tri-anti-tumor-antigen modality expressed in the same iT cell, the combination of i) CAR (CD19, MICA/B, or BCMA), ii) CD16 (hnCD16) and iii) TCR (MR1 or NYESO1) was tested for proof-of-concept. First, the edits were successfully expressed, and all tested combinations were differentiated into functional iT cells. As shown in FIG. 12A, representative FACS plots are gated on live CD45⁺ singlets for TCRαβ and CAR19 expression, demonstrating that both CAR and TCR are successfully expressed in the same iT as compared to TCR null CAR19-iT control. FIG. 12B demonstrates a schedule for daily in vitro restimulation and sampling assay. Using CAR with MR1-TCR for the 9-day serial killing assay, iT cells and target were setup at a 1 to 1 ratio, and targets were added to the culture from day 0 to day 9. Sampling was performed everyday and precise tumor count was obtained via FACS counting beads. A prompt tumor growth inhibition (TGI) mediated by CAR only was observed (FIG. 12C), while the tumor cell killing mediated by TCR only lagged behind the CAR. All conditions (CAR, TCR, or CAR+TCR) demonstrated nearly complete TGI after day 5. Notably, with co-expressed CAR and TCR, the initiation of TGI and completion thereof both took place at a much faster rate, and the killing effect of the cells are long lasting.

It was shown that expressing CAR and TCR on the same iT cell can function both independently and synergistically on tumor control and for T cell activation, as further demonstrated by CD25 upregulation. As shown in FIG. 12D, co-triggering of CAR and TCR revealed the most rapid CD25 upregulation at the highest sustainable level indicating compatibility and a synergistic effect between CAR and TCR expressed in the same effector cell. Using iT cells expressing MICAS-CAR and hnCD16, the cell functions, including cell proliferation through CD3ζ signaling in the presence of CAR, CD16-mediated antigen-specific killing, and cytokine release, were confirmed, indicating the compatibility between CAR and hnCD16.

As shown in FIG. 13A, CAR (CAR-BCMA), TCR (TCR-MR1) and CD16 (hnCD16) are successfully expressed in the same iT (Tri-modal iT) cell as compared to CAR19-iT cell control (neg ctrl). The same serial killing assay as described above was performed to demonstrate the additive killing and synergistic effects among CAR, TCR, and CD16 (Tri-modal iT cells). As shown in FIG. 13B, the tumor count relative to day 0, from day 0 to day 9 for all groups, was obtained via FACS counting beads. Triggering of hnCD16 via therapeutic antibody reveals a similar TGI pattern to TCR with a delayed onset but persistent TGI. Co-triggering of CAR-specific killing, TCR-specific killing, and CD16-mediated ADCC functions in the presence of a therapeutic antibody demonstrated the rapid, deep and durable TGI among all tested groups.

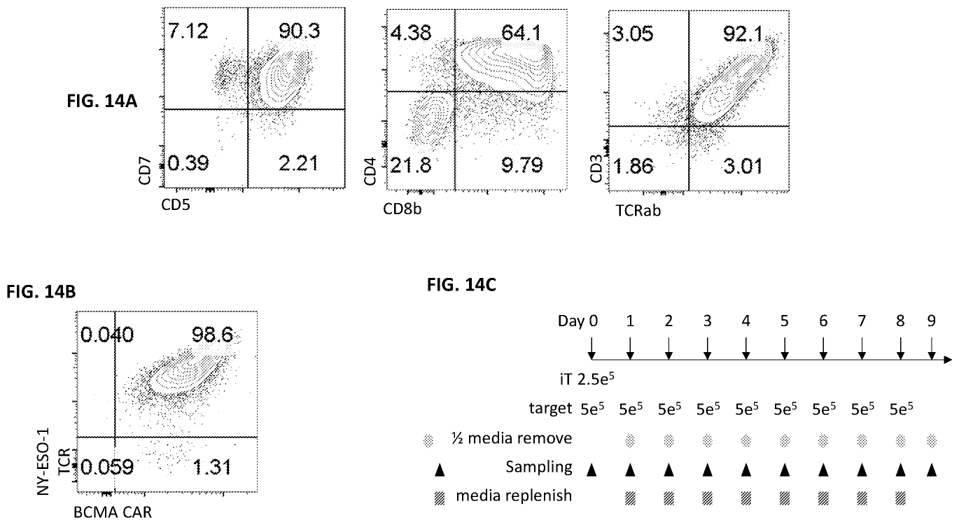

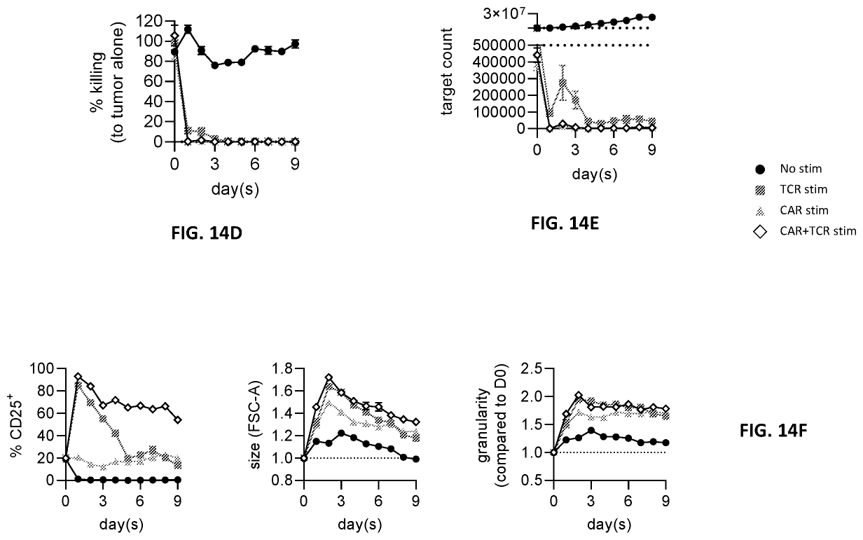

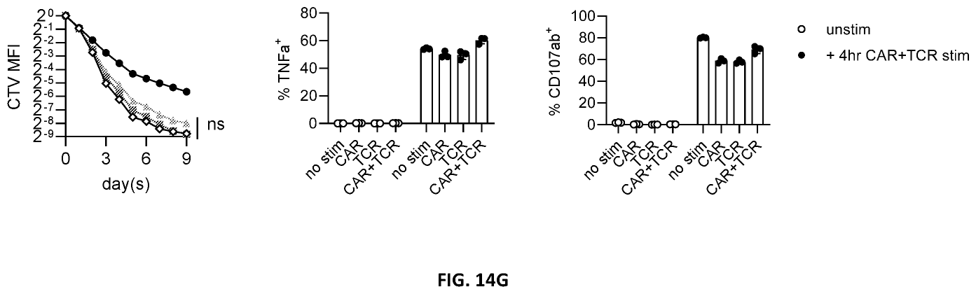

To test applicability of a TCR with a different tumor antigen specificity in combination with CAR and CD16, iPSCs and effector cells were generated to comprise the combination of i) CAR (BCMA), ii) CD16 (hnCD16) and iii) NYESO1-TCR, and various control cells were made in a similar manner. As shown in FIG. 14A, representative FACS plots demonstrate CD7, CD5, CD4, CD8b, surface CD3, and TCRαβ expression of BCMA-CAR⁺ NYESO1-TCR⁺ iT cells on D35, and confirmation of successful expression of both CAR and TCR in the same iT at D42 is shown in FIG. 14B. FIG. 14C presents a schedule for an in vitro restimulation and sampling assay. In this 9-day serial killing assay, iT cells expressing both BCMA-CAR and NYESO1-TCR and target were set up at a 1:2 E:T ratio, and targets were added to the culture from day 0 to day 8. Sampling was performed everyday and precise tumor count was obtained via FACS counting beads. Similar to the observation made with the MR1-TCR and CAR combination, a prompt tumor growth inhibition (TGI) mediated by CAR only was observed (FIG. 14D), while the tumor cell killing mediated by TCR only lagged behind the CAR, thereby confirming that CAR and TCR on the same iT can function both independently and synergistically in an antigen-specific manner. All conditions (CAR, TCR, or CAR+TCR) demonstrated nearly complete TGI after day 3. In addition, CAR⁺ TCR⁺ iT demonstrated efficient and prolonged killing of daily added targets (500,000-100,000 count) (FIG. 14E). As above, co-triggering of CAR and TCR revealed the most rapid CD25 upregulation at the highest sustainable level, in addition to increase in effector size and granularity indicating effector cell fitness and activation (FIGS. 14F and 14G). All together, the compatibility and the synergistic effect between CAR and TCR when expressed in the same effector cell proved that the tri-modal iT cells are an improved cell platform conveying enhanced cell function in tumor cell control and clearance.

### Example 7— Expression of an Exogenous TCR Enhances Efficacy of CAR-Induced Tumor Killing Even without Activation by TCR Ligand

To evaluate whether exogenous NY-ESO-1 TCR expression in tri-modal iT cells also affects efficacy and assess antigen-specific tumor killing efficacy of NY-ESO-1 TCR CAR iT effectors, iPSC having TRBC2 knockout and a biallelic insertion of BCMA-CAR and hnCD16 at TRAC were generated (TCR^{neg}/CAR/hnCD16 iPSC) as a source for effector cell derivation. During iT differentiation (around D10), the differentiating iPS cells were further transduced with NYSEO1-TCR, resulting effector T cells having TCR^{neg}/CAR/hnCD16/NYSEO1-TCR at the end of the differentiation process (around D42) for cryopreservation, culturing and further evaluation.

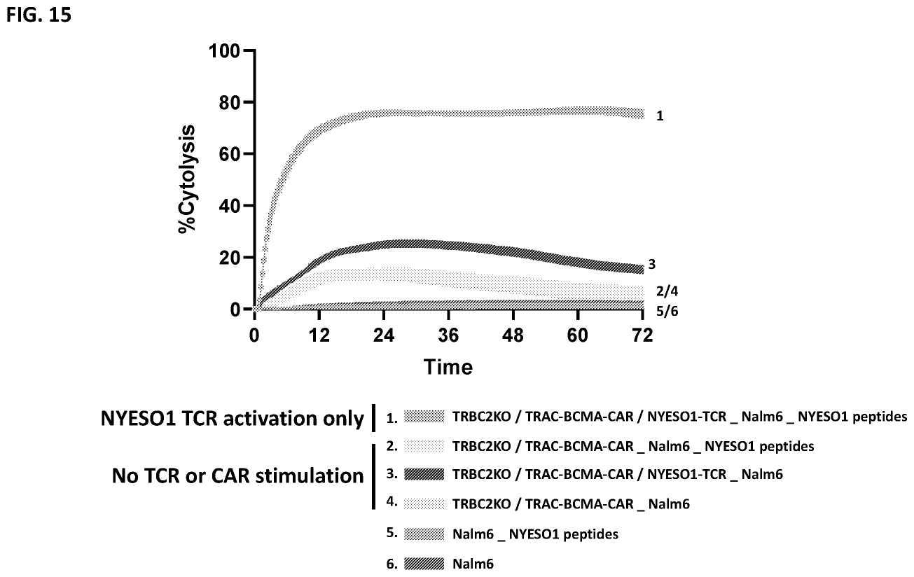

A CR/D response assay was designed to use different combinations of: (i) effector cells (CAR expression with or without the exogenous TCR expression), (ii) tumor targets (with or without the CAR antigen) and (iii) the presence or absence of a TCR ligand specific to the exogenous TCR of the effector cell, in order to provide the effector cells in the assay either no stimulation, TCR activation only, CAR activation only, or both TCR and CAR activation. With such a design, the effector cells were tested for TCR antigen-specific tumor killing efficacy, CAR-induced tumor killing efficacy, separately and in combination. The E:T ratio in all assays was about 1:3, and the combinations of the effector cell, tumor target, and stimulation are as indicated in each of FIGS. 15-18.

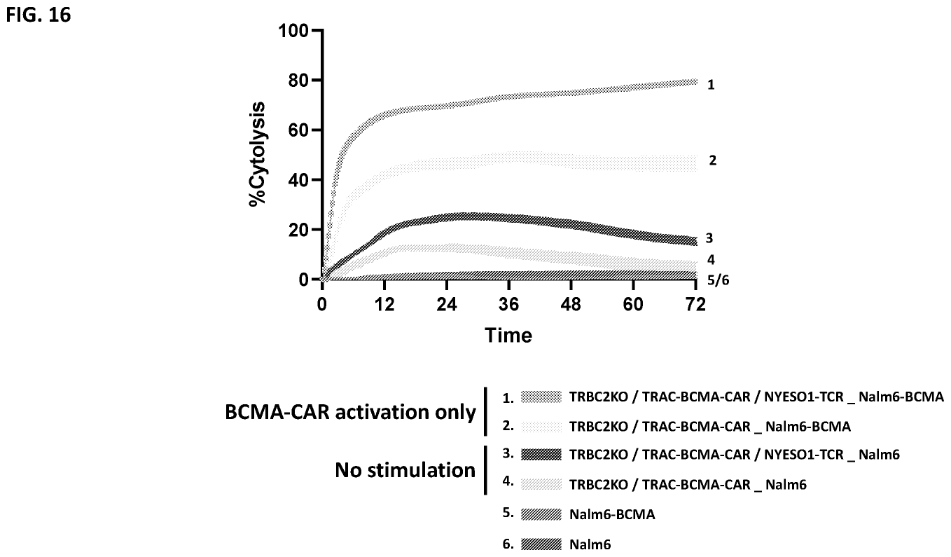

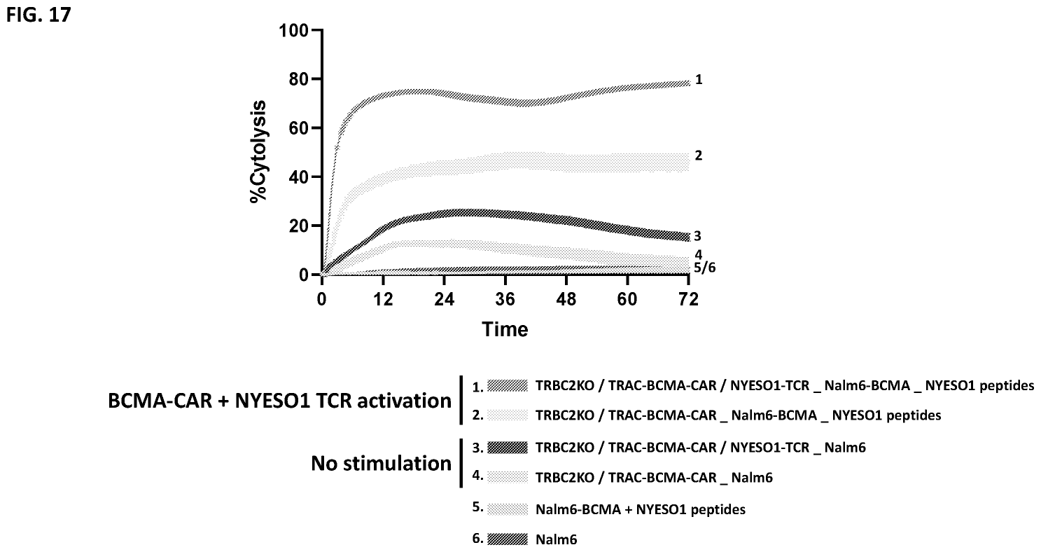

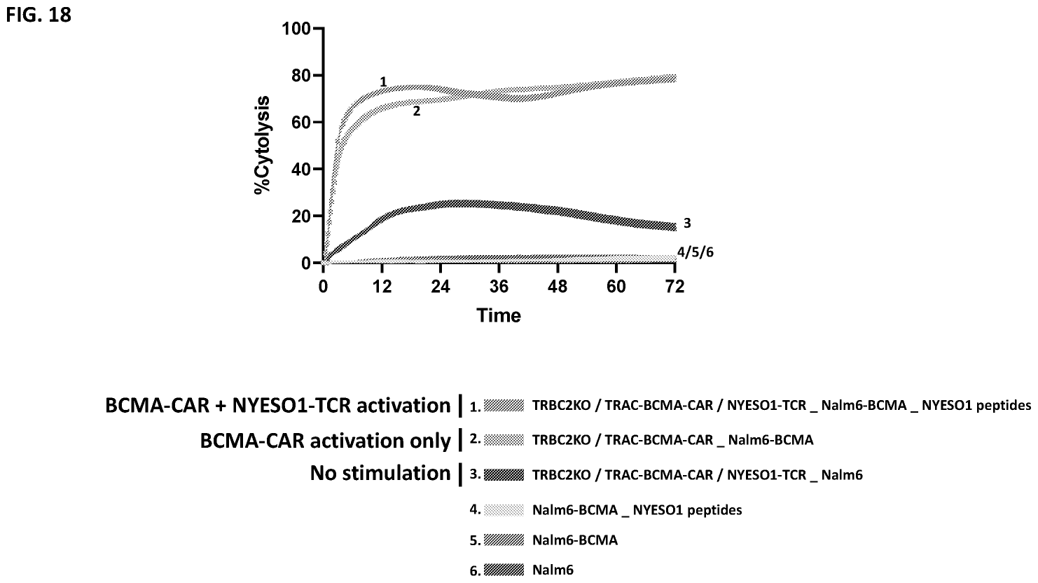

With only TCR being activated in a CAR/TCR effector cell, it is shown in FIG. 15 that the exogenous TCR expressed in iT effector cells is capable of triggering TCR antigen-specific tumor killing. Interestingly, the mere expression of the exogenous TCR, even without being activated for lack of a matching TCR ligand, enhances CAR-induced tumor killing efficacy in CAR-iT effector cells, as demonstrated by comparing the Nalm 6 (BCMA⁺) tumor cell killing by BCMA-CAR iT cells and TCR expressing BCMA-CAR iT cells in the absence of a TCR ligand in FIG. 16. When in the presence of both CAR and TCR tumor antigens, effector cell expressing both CAR and TCR with respective tumor antigen specificity shows enhanced tumor killing efficacy compared to effector cell expressing only CAR (FIG. 17). Another observation from this comprehensively and elegantly designed assay and data analyses is that the activation of both TCR and CAR leads to faster and/or rapid tumor killing compared to CAR activation alone (FIG. 18).

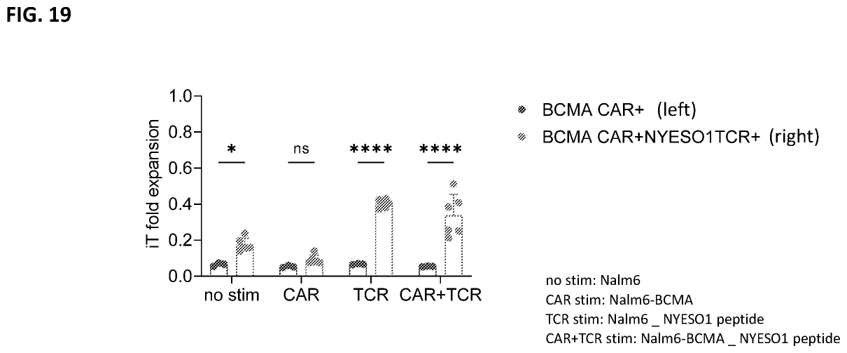

In addition, the exogenous TCR-expressing CAR effector cells showed higher fold expansion compared to CAR effector cells without the TCR expression under all stimulation conditions (i.e., no stimulation, CAR stimulation only, TCR stimulation only, or both CAR and TCR stimulations), with the highest iT cell expansion in the cells that are stimulated by only TCR signaling without CAR activation (FIG. 19). Also shown in FIG. 19, CAR activation alone has the lowest iT cell expansion, maybe due to CAR induced cell exhaustion. Indeed, with CAR activation in addition to TCR activation, the iT cell fold expansion reduces as compared to that under TCR activation alone. Without being limited by theory, the exogenous TCR expression and/or activation in CAR-iT cells may be associated with better persistence and cell fitness upon tumor antigen stimulation.

### Example 8— In Vivo Validation of Tri-Modal iT Cells

To evaluate the function of the tri-modal iT cells in vivo, Nalm6 expressing luciferase (Nalm6-luc), a mouse xenograft model of B cell leukemia, was used. The tri-modal iT cell in this particular test expresses BCMA-CAR, NYESO-TCR, and CD16. Each individual edit was successfully triggered by its matching stimulant, with hnCD16 mediated ADCC being activated by an IgG antibody.

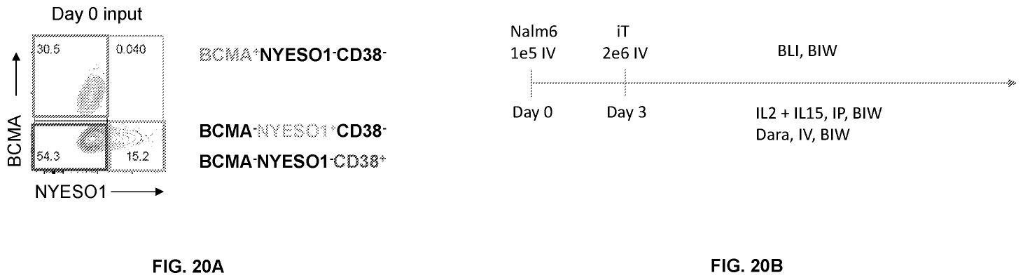

To evaluate the effect of combining CAR, TCR and CD16-mediated ADCC, Nalm6-luc tumor cell lines that express cell surface BCMA, NYESO1 or CD38 for CAR, TCR, or anti-CD38 antibody targeting, respectively, were generated and mixed to provide a heterogenous tumor cell population. In this set of assays, the anti-CD38 antibody used was daratumumab. To evaluate the additive effect of CAR, TCR and CD16, Nalm6-luc lines were generated to specifically express the desired antigen for either CAR, TCR, or ADCC. Different cell lines were mixed at a 1:1:1 ratio to provide a total of 1×10⁵ of heterogeneous tumor cells, which were injected to mice on day 0 (FIG. 20A). As illustrated in FIG. 20B, 2×10⁶ of indicated iT cells (BCMA-CAR, NYESO1-TCR, CD16, and CD38 null) were injected on day 3. Daratumumab (Dara) at 1.5 mg/kg (IV, intravenously), and IL2 (100KU) and IL15 (150 ng) cytokine support (IP, intraperitoneally) were administered twice per week (BIW). Bioluminescence imaging (BLI) was recorded twice per week.

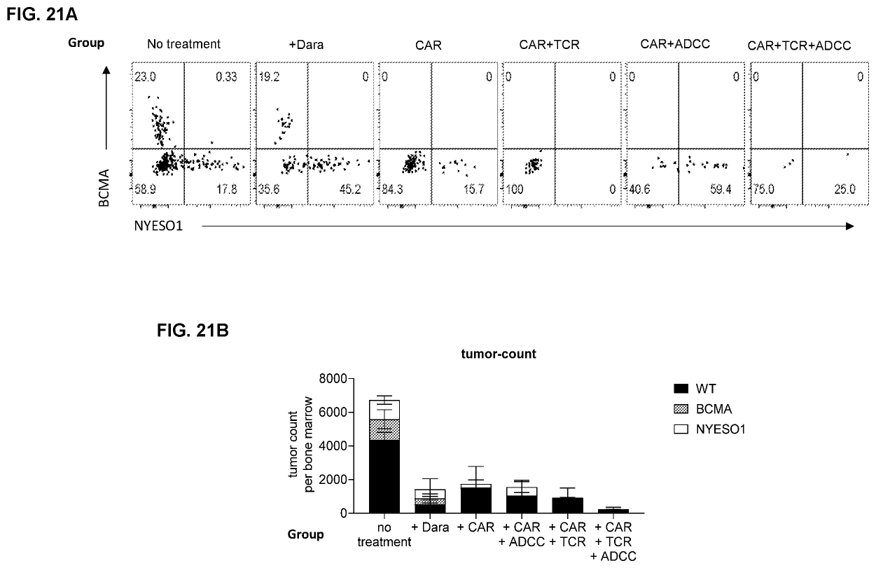

The tumor cell elimination in the heterogenous population as a result of being targeted by indicated iT cells was assessed by FACS analysis of ex vivo CD10⁺ cells from bone marrow (BM) for BCMA-mCherry and NYESO1-GFP signals. As shown, with no treatment or with Dara alone, the presence of cells of all three lines is detected by FACS; while under targeting of iT cells having BCMA-CAR, BCMA-expressing tumor cells in the mixed population are eliminated; and under the targeting of TCR, the NYESO1-expressing cells are eliminated; and the tumor cells had the least escape when the heterogeneous tumor cell population is targeted concertedly by combined CAR, TCR and ADCC targeting specificity in the effector cells (FIG. 21A). Further, the absolute tumor count in each bone marrow sample for indicated tumor lines for all groups is shown in FIG. 21B.

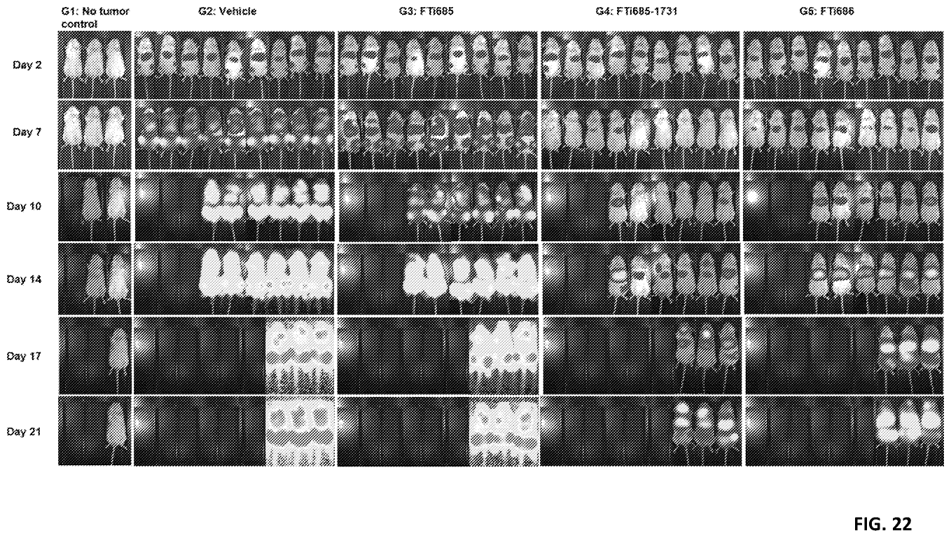

The twice weekly recorded bioluminescence images (BLI) are included in FIG. 22, showing that CAR+TCR iT cleared majority of tumors in bone marrow by day 7 and 10, but tumors in lung and brain area were not cleared or under control.

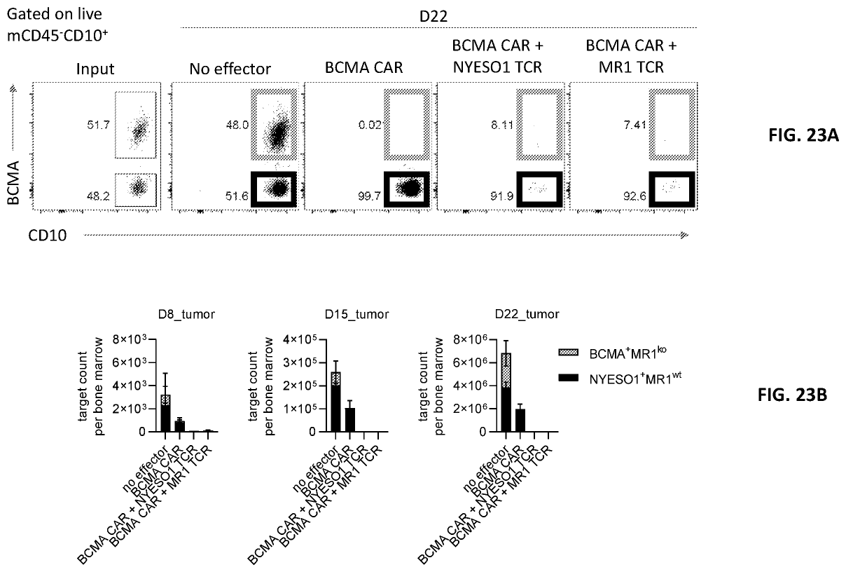

Representative FACS plot in FIG. 23A shows the ratio between CAR-targeted Nalm6 (BCMA⁺) and TCR-targeted Nalm6 (BCMA⁻) of input, and the analysis of Day 22 bone marrow cell suspension from no effector, BCMA CAR alone, BCMA-CAR⁺NYESO1-TCR⁺ and BCMA-CAR⁺MR1-TCR⁺ groups. The absolute tumor count in each bone marrow sample for indicated and for individual groups is plotted in FIG. 23B for BCMA⁺MR1^{ko} and NYESO1⁺MR1^{wt} Nalm6. These tumor counts in bone marrow confirmed the antigen-specific in vivo efficacy of CAR and TCR co-expressing iT cells for various CAR and TCR antigen combinations.

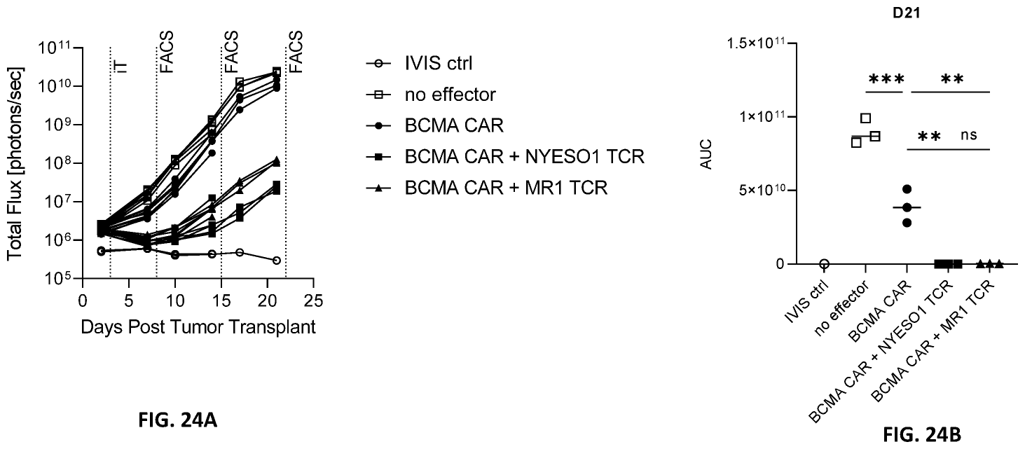

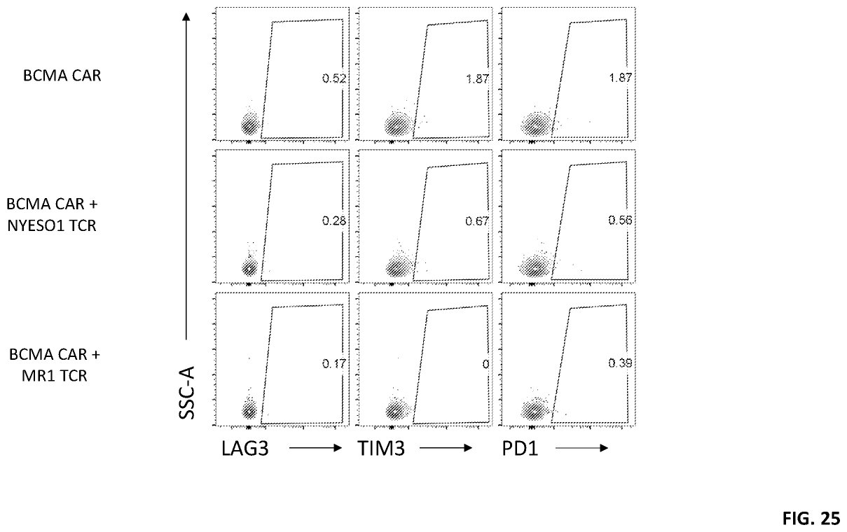

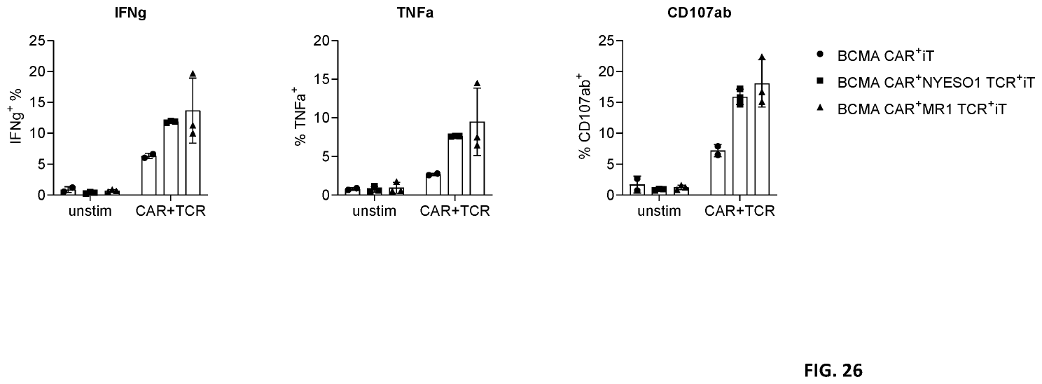

BLI (FIG. 24A) and the Area under curve (AUC) data (FIG. 24B) confirmed significantly better TGI in mice treated with CAR⁺ TCR⁺ iT cells, with TCR targeting specificity being NYESO1 or MR1. Further, FACS analysis demonstrates the absence of checkpoint inhibitory receptors, LAG3, TIM3, PD1 on ex vivo iT cells from indicated groups, which indicates that the iT cells were not exhausted by Day 22 (FIG. 25). In addition, CD45⁺ iT cells were enriched using magnetic beads from a single cell suspension of bone marrow on day 22. The enriched cells were divided into two wells, one for unstim control and the other one was stimulated with Nalm6 MR1^{wt} expressing BCMA in the presence of NYESO1 peptide. GolgiStop™, Golgiblock™ and CD107a and CD107b antibodies were added to all samples. After 4 hrs, samples were stained for surface proteins, live/dead and fixed/permed for intracellular staining. The cytokine production and degranulation capacity of ex vivo iT cells from bone marrow on day 22 as shown in FIG. 26 confirmed that the effector cells were still functional.

The data presented herein therefore demonstrate the compatibility and enhanced functionality between CAR, TCR and CD16-mediated ADCC in mitigating tumor escape resulting from tumor heterogeneity by both in vitro and in vivo tumor models, providing a strategy of off-the-shelf cellular immunotherapy in combating heterogenous and difficult to treat solid tumors.

### Example 9— CAR Design as an Alternative to Antigen Specific TCR

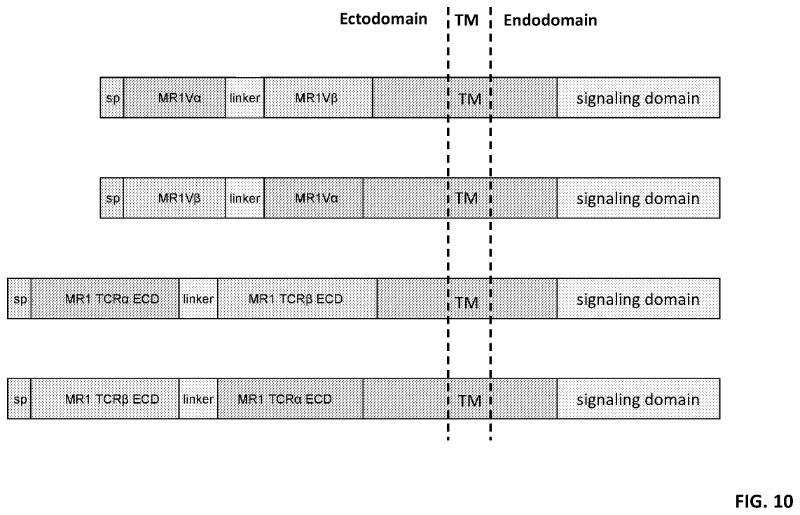

To evaluate whether a CAR can be designed to target the same tumor associated antigen as a recombinant TCR (or exogenous TCR), the MR1-TCR is used for proof-of-concept. In the case of MR1, the transgenic TCRα chain and the transgenic TCRβ chain form the exogenous TCR complex that mediates the MR1-specific TCR signaling and cytotoxicity, in addition to reconstituting cell surface CD3 that is useful for CD3-based engagers. As shown in FIG. 10, in one group of the CAR designs, the ectodomain of the CAR is comprised of the variable alpha fragment of a transgenic TCRα chain (Vα) and the variable beta fragment of a transgenic TCRβ chain (Vβ), linked in any order. Another group of CAR designs as demonstrated in FIG. 10 utilizes the extracellular domain of the transgenic TCRα chain (TCRα ECD) and the transgenic TCRβ chain (TCRβ ECD), such that the binding region of the CAR is comprised of the variable region and a partial constant region of the transgenic TCRα chain and the variable region and a partial constant region of the transgenic TCRβ chain.

In the context of MR1-TCR, the MR1 Vα (variable alpha (Vα) fragment of MR1) is represented by a sequence that has at least 90% identity to SEQ ID NO: 21; the MR1 VP (variable alpha (Vβ) fragment of MR1) is represented by a sequence that has at least 90% identity to SEQ ID NO: 22; the MR1 TCRα ECD is represented by a sequence that has at least 90% identity to SEQ ID NO: 23; and the MR1 TCRβ ECD is represented by a sequence that has at least 90% identity to SEQ ID NO: 24:

SEQ ID NO: 21
MACPGFLWALVISTCLEFSMAQTVTQSQPEMSVQEAETVT
LSCTYDTSESDYYLFWYKOPPSRQMILVIRQEAYKQQNAT
ENRFSVNFQKAAKSFSLKISDSQLGDAAMYFCAYRSAVNA
RLMFGDGTQLVVKPN
(the underlined signal peptide is flexible
in length and sequence; same below)
SEQ ID NO: 22
MTIRLLCYVGFYFLGAGLMEADIYQTPRYLVIGTGKKITL
ECSQTMGHDKMYWYQQDPGMELHLIHYSYGVNSTEKGDLS
SESTVSRIRTEHFPLTLESARPSHTSQYLCASSEARGLAE
FTDTQYFGPGTRLTVLE
SEQ ID NO: 23
MACPGFLWALVISTCLEFSMAQTVTQSQPEMSVQEAETVT
LSCTYDTSESDYYLFWYKOPPSRQMILVIRQEAYKQQNAT
ENRFSVNFQKAAKSFSLKISDSQLGDAAMYFCAYRSAVNA
RLMFGDGTQLVVKPNIQNPDPAVYQLRDSKSSDKSVCLFT
DFDSQTNVSQSKDSDVYITDKCVLDMRSMDFKSNSAVAWS
NKSDFACANAENNSIIPEDTFFPSPESSCDVKLVEKSFET
DTNLNFQNLS
SEQ ID NO: 24
MTIRLLCYVGFYFLGAGLMEADIYQTPRYLVIGTGKKITL
ECSQTMGHDKMYWY QQDPGMELHLIHYSYGVNSTEKGDL
SSESTVSRIRTEHFPLTLESARPSHTSQYLCASSEARGLA
EFTDTQYFGPGTRLTVLEDLKNVFPPEVAVFEPSEAEISH
TQKATLVCLATGFYPDHVELSWWVNGKEVHSGVCTDPQPL
KEQPALNDSRYCLSSRLRVSATFWQNPRNHERCQVQFYGL
SENDEWTQDRAKPVTQIVSAEAWGRADCGFTSESYQQGVL
SA

For testing purposes, the transmembrane domain and the cytoplasmic domain of the CAR incorporated CD28 and CD3 ζ1XX. The designed CARs are each expressed in primary NK and T cells for examining cell specific surface expression profiles. Each CAR construct in FIG. 10 further contains a Thy1.1 marker at the C-terminus, which is separated from the construct by a 2A peptide (not shown). On about day 10 following transduction, the transduced cells are assayed for CAR and Thy1.1 expression by FACS. Successfully transduced cells are sorted based on Thy1.1 expression, whereas CAR staining was performed with an antibody specific for the antigen binding region of the CAR.

To show antigen-specific killing mediated by the CAR candidates, the MR1-CAR NK or T cells are co-cultured with tumor cells that express MR1 or are MR1 null or low, using cells having no MR1-CAR as negative controls. Each MR1-CAR demonstrating specific killing ability is then transduced to iPSC. All MR1-CAR iPSC lines are examined for CAR expression, karyotype abnormality, and genome stability. With or without expression in iPSC, each MR1-CAR-iPSC line is carried on for both T cell and NK cell differentiation according to the methods described herein. Day 10 and Day 20 intermediary cells, and cells at other time points during differentiation are characterized for marker expression profile and cell growth. Cell expansion at key time points and at the end of the differentiation process are also evaluated.

To determine the function profile of derivative effector cells expressing a MR1-CAR, the effector cells and MR1 expressing tumor cell line cells (target cell) are co-cultured. Within the same co-culture conditions, MR1-CAR effector cell activation is examined by production of cytokines IFNγ and TNFα, degranulation by assessment of surface CD107a, and direct killing of the target cell lines using a caspase-based flow assay. Increased levels of cytokine and degranulation and an increase in direct killing by MR1-CAR effector cells versus unmodified effector cells in response to MR1 positive target cells, compared to no observed difference in activity when co-cultured with MR1 negative targets demonstrates MR1-CAR effector cell activation and cytotoxicity in the presence of MR1 cell surface antigen.

In vivo function of MR1-CAR is evaluated using human-MR1 expressing cancer cell lines as targets. For in vivo evaluation, the mouse or human T cells are transplanted into immunocompromised NSG mice. Delayed tumor progression, induced tumor regression, and/or prolonged survival are assessed in the treatment of NSG mice bearing any of the tumor types using MR1-CAR expressing primary or derivative effector cells.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A cell or a population thereof, wherein the cell is an eukaryotic cell, an animal cell, a human cell, an immune cell, an induced pluripotent cell (iPSC), a clonal iPSC or a derivative cell differentiated therefrom, and wherein the cell comprises:
(i) a polynucleotide encoding a transgenic TCRα chain (tgTCRα); and
(ii) a polynucleotide encoding a transgenic TCRβ chain (tgTCRβ), wherein the tgTCRα chain and the tgTCRβ chain form an exogenous TCR complex (TCR^{exo}) that recognizes a first tumor antigen; and optionally,
(iii) one or more additional exogenous polynucleotides comprising a polynucleotide encoding a chimeric antigen receptor (CAR) or an engager targeting at least a second tumor antigen.

2. The cell or population thereof of claim 1, wherein:
(i) the polynucleotide encoding the tgTCRα chain and the polynucleotide encoding the tgTCRβ chain are comprised in a bi-cistronic construct, and optionally wherein:
(a) the construct is inserted at a constant region of TCRα or TCRβ (TRAC or TRBC);
(b) the insertion of the construct disrupts expression of endogenous TCRα or TCRβ at the insertion site; and/or
(c) expression of the construct is driven by an endogenous promoter of TCR or an exogenous promoter; or
(ii) the polynucleotide encoding the CAR or the engager is inserted at TRAC or TRBC, and optionally wherein:
(a) the insertion of the polynucleotide encoding the CAR or the engager disrupts expression of the endogenous TCRα or TCRβ at the insertion site; and/or
(b) expression of the CAR or the engager is driven by an endogenous promoter of TCR or an exogenous promoter; or
(iii) the polynucleotides encoding the tgTCRα chain and the tgTCRβ chain, the CAR or the engager, or the one or more additional polynucleotides are inserted in one, or more safe harbor loci or selected gene loci.

3. The cell or population thereof of claim 2, (I) wherein the construct and the polynucleotide encoding the CAR or the engager are each inserted at a constant region of TCRα or TCRβ (TRAC or TRBC), but not at the same constant region, thereby disrupting expression of both endogenous TCRα and TCRβ, knocking out the endogenous TCR, and avoiding a mis-paired TCR comprising:
(a) the transgenic TCRα and the endogenous TCRβ, or
(b) the transgenic TCRβ and the endogenous TCRα;
or (II) wherein the construct and the polynucleotide encoding the CAR or the engager are each integrated at a locus comprising a safe harbor locus or a selected gene locus.

4. The cell or population thereof of claim 3, wherein:
(i) the safe harbor locus comprises at least one of AAVS1, CCR5, ROSA26, collagen, HTRP, H11, GAPDH, or RUNX1;
(ii) the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CD69, CD71, CIS, CBL-B, SOCS2, PD1, CTLA4, LAG3, TIM3, or TIGIT; and/or
(iii) the integration of the exogenous polynucleotides knocks out expression of the gene in the locus.

5. The cell or population thereof of claim 1, wherein the first tumor antigen and the second tumor antigen each comprises at least one of:
(i) MR1, NYESO1, MICA/B, EpCAM, EGFR, B7H3, Muc1, Muc16, CD19, BCMA, CD20, CD22, CD38, CD123, HER2, CD52, GD2, MSLN, VEGF-R2, PSMA and PDL1; or
(ii) ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NYESO1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; and
wherein the first tumor antigen and the second tumor antigen are the same or different.

6. The cell or population thereof of claim 1, wherein the first tumor antigen comprises at least one of MR1, NYESO1, and MICA/B; or
wherein:
(i) the tgTCRα comprises a variable alpha (Vα) fragment that has at least about 85% identity to SEQ ID NO: 7, and a TCRα constant fragment comprising a sequence having at least about 85% identity to SEQ ID NO: 8; and/or
(ii) the tgTCRβ comprises a variable alpha (Vβ) fragment that has at least about 85% identity to SEQ ID NO: 9, and a TCRβ constant fragment comprising a sequence having at least about 85% identity to SEQ ID NO: 10.

7. The cell of population thereof of claim 1, wherein the CAR is:
(i) T cell specific or NK cell specific;
(ii) a bi-specific antigen binding CAR;
(iii) a switchable CAR;
(iv) a dimerized CAR;
(v) a split CAR;
(vi) a multi-chain CAR;
(vii) an inducible CAR;
(viii) an inactivation CAR;
(ix) co-expressed with a partial or full peptide of a cell surface expressed exogenous cytokine and/or a receptor thereof, optionally in separate constructs or in a bi-cistronic construct;
(x) co-expressed with a checkpoint inhibitor, optionally in separate constructs or in a bi-cistronic construct.

8. The cell or population thereof of claim 1, wherein the engager comprises:
(i) a first binding domain recognizing an extracellular portion of CD3, CD28, CD5, CD16, CD64, CD32, CD33, CD89, NKG2C, NKG2D, or any functional variants thereof of the cell or a by-stander immune effector cell; and
(ii) a second binding domain targeting the second tumor antigen that is different from the first tumor antigen targeted by the exogenous TCR, and wherein the second binding domain of the engager is specific to any one of: B7H3, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2.

9. The cell or population thereof of any one of claims 1-8, wherein the cell further comprises one or more of:
(i) CD38 knockout;
(ii) HLA-I deficiency and/or HLA-II deficiency;
(iii) introduced HLA-G or non-cleavable HLA-G, or knockout of one or both of CD58 and CD54;
(iv) a CD16 or a variant thereof;
(v) a chimeric fusion receptor (CFR);
(vi) a signaling complex comprising a partial or full peptide of a cell surface expressed exogenous cytokine and/or a receptor thereof;
(vii) at least one of the genotypes listed in Table 1;
(viii) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(ix) introduction or upregulation of at least one of HLA-E, 4-1BBL, CD3, CD4, CD8, CD16, CD47, CD113, CD131, CD137, CD80, PDL1, A_{2A}R, Fc receptor, an antibody or functional variant or fragment thereof, a checkpoint inhibitor, and surface triggering receptor for coupling with an agonist.

10. The cell or population thereof of claim 9, wherein the CD16 or a variant thereof comprises at least one of:
(a) a high affinity non-cleavable CD16 (hnCD16);
(b) F176V and S197P in ectodomain domain of CD16;
(c) a full or partial ectodomain originated from CD64;
(d) a non-native (or non-CD16) transmembrane domain;
(e) a non-native (or non-CD16) intracellular domain;
a non-native (or non-CD16) signaling domain;
(g) a non-native stimulatory domain; and
(h) transmembrane, signaling, and stimulatory domains that are not originated from CD16, and are originated from a same or different polypeptide.

11. The cell or population thereof of claim 9, wherein the CFR comprises an ectodomain fused to a transmembrane domain, which is operatively connected to an endodomain, and wherein the ectodomain, transmembrane domain and the endodomain do not comprise any endoplasmic reticulum (ER) retention signals or endocytosis signals.

12. The cell or population thereof of claim 11, wherein:
(i) the ectodomain of the CFR comprises a full or partial length of an extracellular portion of a signaling protein comprising at least one of CD3ε, CD3γ, CD3δ, CD28, CD5, CD16, CD64, CD32, CD33, CD89, NKG2C, NKG2D, any functional variants, and a combination or a chimera thereof;
(ii) the ectodomain of the CFR initiates signal transduction upon binding to a selected agonist; or
(iii) the endodomain of the CFR comprises a cytotoxicity domain comprising at least a full length or a portion of CD3ζ, 2B4, DAP10, DAP12, DNAM1, CD137 (4-1BB), IL21, IL7, IL12, IL15, NKp30, NKp44, NKp46, NKG2C, or NKG2D polypeptide; and optionally wherein the endodomain further comprises one or more of:
(a) a co-stimulatory domain comprising a full length or a portion of CD2, CD27, CD28, CD40L, 4-1BB, OX40, ICOS, PD-1, LAG-3, 2B4, BTLA, DAP10, DAP12, CTLA-4, or NKG2D polypeptide, or any combination thereof;
(b) a co-stimulatory domain comprising a full length or a portion of CD28, 4-1BB, CD27, CD40L, ICOS, CD2, or combinations thereof;
(c) a persistency signaling domain comprising a full length or a portion of an endodomain of a cytokine receptor comprising IL7R, IL15R, IL18R, IL12R, IL23R, or combinations thereof; and/or
(d) a full or a partial intracellular portion of a receptor tyrosine kinase (RTK), a tumor necrosis factor receptor (TNFR), an EGFR or a FAS receptor.

13. The cell or population thereof of claim 12, wherein the selected agonist is (i) an antibody or a functional variant or fragment thereof; or (ii) an engager; and
wherein the selected agonist is encoded by a polynucleotide comprised in the cell or is comprised in a medium comprising the cell or population thereof.

14. The cell or population thereof of claim 9, wherein the cell surface expressed exogenous cytokine or receptor thereof:
(a) comprises at least one of IL2, IL4, IL6, IL7, IL9, IL10, IL11, IL12, IL15, IL18, IL21, and its respective receptor(s); or
(b) comprises at least one of:
(i) co-expression of IL15 and IL15Rα by using a self-cleaving peptide;
(ii) a fusion protein of IL15 and IL15Rα;
(iii) an IL15/IL15Rα fusion protein with intracellular domain of IL15Rα truncated or eliminated;
(iv) a fusion protein of IL15 and membrane bound Sushi domain of IL15Rα;
(v) a fusion protein of IL15 and IL1510;
(vi) a fusion protein of IL15 and common receptor γC, wherein the common receptor γC is native or modified; and
(vii) a homodimer of IL15Rβ,
wherein any one of (b)(i)-(vii) can be co-expressed with a CAR in separate constructs or in a bi-cistronic construct; or
(c) comprises at least one of:
(i) a fusion protein of IL7 and IL7Rα;
(ii) a fusion protein of IL7 and common receptor γC, wherein the common receptor γC is native or modified; and
(iii) a homodimer of IL7Rβ, wherein any one of (c)(i)-(iii) can be co-expressed with a CAR in separate constructs or in a bi-cistronic construct;
and optionally,
(d) is transiently expressed.

15. The cell or population thereof of claim 9, wherein the checkpoint inhibitor is an antagonist to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, and an inhibitory KIR.

16. The cell or population thereof of any one of claims 1-15, wherein the cell has therapeutic properties comprising one or more of:
(i) increased cytotoxicity;
(ii) improved persistency and/or survival;
(iii) enhanced ability in migrating, and/or activating or recruiting bystander immune cells, to tumor sites;
(iv) improved tumor penetration;
(v) enhanced ability to reduce tumor immunosuppression;
(vi) improved ability in rescuing tumor antigen escape;
(vii) controlled apoptosis;
(viii) enhanced or acquired ADCC; and
(ix) ability to avoid fratricide,
in comparison to its counterpart primary cell obtained from peripheral blood, umbilical cord blood, or any other donor tissues without the same genetic edit(s).

17. The cell or population thereof of any one of claims 1-16, wherein the derivative cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, a derivative B lineage cell, or a derivative effector cell having one or more functional features that are not present in a counterpart primary T, NK, NKT, and/or B cell.

18. The cell or population thereof of claim 17, wherein the derivative effector cell is a hematopoietic cell and comprises longer telomeres in comparison to its counterpart primary cell.

19. The cell or population thereof of any one of claims 1-18, wherein the cell comprises one of the genotypes listed in Table 1; or wherein the cell comprises:
(i) (1) a CD19-CAR at TRAC locus, (2) a TRAC knockout, and (3) a MR1-TCR or a NYESO1-TCR, and optionally (4) a TRBC knockout;
(ii) (1) a BCMA-CAR and hnCD16 insertion at TRAC locus, (2) a TRAC knockout, and (3) a MR1-TCR or NYESO1-TCR, and optionally (4) a TRBC knockout; or
(iii) (1) a MICAS-CAR insertion at TRAC locus, (2) a TRAC knockout, (3) a hnCD16 insertion at CD38 locus, (4) a CD38 knockout, and (5) a MR1-TCR or NYESO1-TCR, and optionally (6) a TRBC knockout.

20. A composition comprising the cell or population thereof of any one of claims 1-19.

21. The composition of claim 20, wherein the cell or population thereof comprises the iPSC derivative effector cell, and wherein the composition further comprises one or more therapeutic agents.

22. The composition of claim 21, wherein the one or more therapeutic agents comprise a peptide, a cytokine, a checkpoint inhibitor, an antibody or functional variant or fragment thereof, an engager, a mitogen, a growth factor, a small RNA, a dsRNA (double stranded RNA), mononuclear blood cells, feeder cells, feeder cell components or replacement factors thereof, a vector comprising one or more polynucleic acids of interest, a chemotherapeutic agent or a radioactive moiety, or an immunomodulatory drug (JIVED).

23. The composition of claim 22, wherein:
(a) the checkpoint inhibitor comprises:
(i) one or more antagonist checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or an inhibitory KIR;
(ii) one or more of atezolizumab, avelumab, durvalumab, ipilimumab, IPH4102, IPH43, IPH33, lirimumab, monalizumab, nivolumab, pembrolizumab, and their derivatives or functional equivalents; or
(iii) at least one of atezolizumab, nivolumab, and pembrolizumab; or
(b) the one or more therapeutic agents comprise one or more of venetoclax, azacitidine, and pomalidomide.

24. The composition of claim 22, wherein the antibody, or functional variant or fragment thereof comprises:
(a) anti-CD20, anti-CD22, anti-HER2, anti-CD52, anti-EGFR, anti-CD123, anti-GD2, anti-PDL1, and/or anti-CD38 antibody;
(b) one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or
(c) daratumumab, and wherein the derivative effector cell comprises a CD38 knockout, and optionally expresses CD16 or a variant thereof.

25. The composition of claim 22, wherein the engager comprises:
(i) a bispecific T cell engager (BiTE);
(ii) a bispecific killer cell engager (BiKE); or
(iii) a tri-specific killer cell engager (TriKE); or
wherein the engager comprises:
(a) a first binding domain recognizing an extracellular portion of CD3, CD28, CD5, CD16, CD64, CD32, CD33, CD89, NKG2C, NKG2D, or any functional variants thereof of the cell or a by-stander immune effector cell; and
(b) a second binding domain specific to an antigen comprising any one of: B7H3, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2.

26. Therapeutic use of the composition of any one of claims 20-25 by introducing the composition to a subject suitable for adoptive cell therapy, wherein the subject has an autoimmune disorder, a hematological malignancy, a solid tumor, cancer, or a viral infection.

27. A master cell bank (MCB) comprising the clonal iPSC of any one of the claims 1-19.

28. A method of manufacturing a derivative effector cell of any one of the claims 1-19 comprising:
differentiating a genetically engineered iPSC, wherein the iPSC comprises: (a) a polynucleotide encoding a transgenic TCRα chain (tgTCRα); (b) a polynucleotide encoding a transgenic TCRβ chain (tgTCRβ); and optionally, (c) a polynucleotide encoding a chimeric antigen receptor (CAR) or an engager targeting a second tumor antigen; and optionally wherein the iPSC further comprises one or more of:
(i) CD38 knockout;
(ii) HLA-I deficiency and/or HLA-II deficiency;
(iii) introduced HLA-G or non-cleavable HLA-G, or knockout of one or both of CD58 and CD54;
(iv) a CD16 or a variant thereof;
(v) a chimeric fusion receptor (CFR);
(vi) a signaling complex comprising a partial or full peptide of a cell surface expressed exogenous cytokine and/or a receptor thereof;
(vii) at least one of the genotypes listed in Table 1;
(viii) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(ix) introduction or upregulation of at least one of HLA-E, 4-1BBL, CD3, CD4, CD8, CD16, CD47, CD113, CD131, CD137, CD80, PDL1, A_{2A}R, Fc receptor, an antibody or functional variant or fragment thereof, a checkpoint inhibitor, and surface triggering receptor for coupling with an agonist.

29. The method of claim 28, further comprising:
genomically engineering a clonal iPSC to knock in: (a) the polynucleotide encoding the transgenic TCRα chain (tgTCRα); (b) the polynucleotide encoding the transgenic TCRβ chain (tgTCRβ); and optionally, (c) the polynucleotide encoding the chimeric antigen receptor (CAR) or the engager targeting the second tumor antigen; and optionally further comprising genomically engineering the clonal iPSC:
(i) to knock out CD38,
(ii) to knock out B2M and/or CIITA,
(iii) to knock out one or both of CD58 and CD54, and/or
(iv) to introduce HLA-G or non-cleavable HLA-G, the CD16 or a variant thereof, the CFR, and/or the signaling complex comprising the partial or full peptide of the cell surface expressed exogenous cytokine and/or receptor thereof.

30. The method of claim 29, wherein the genomic engineering comprises targeted editing.

31. The method of claim 30, wherein the targeted editing comprises deletion, insertion, or in/del, and wherein the targeted editing is carried out by CRISPR, ZFN, TALEN, homing nuclease, homology recombination, or any other functional variation of these methods.

32. A chimeric antigen receptor (CAR) specific to tumor cell surface antigen MR1, wherein the MR1-CAR comprises:
(i) an ectodomain comprising at least one antigen recognition domain, wherein the antigen recognition domain comprises:
(a) a variable alpha (Vα) fragment that has at least about 85% identity to SEQ ID NO: 7 (MR1Vα), and a variable beta (Vβ) fragment that has at least about 85% identity to SEQ ID NO: 8 (MR1Vβ); or
(b) an extracellular domain of MR1 TCRα that has at least about 85% identity to SEQ ID NO: 15, and an extracellular domain of MR1 TCRβ that has at least about 85% identity to SEQ ID NO: 16;
(ii) a transmembrane domain; and
(iii) an endodomain comprising at least a first signaling domain, wherein the first signaling domain is originated from a cytoplasmic domain of a signal transducing protein specific to T and/or NK cell activation or functioning; and
wherein the tumor cell surface antigen MR1 is non-polymorphic.

33. The chimeric antigen receptor of claim 32, wherein the signal transducing protein comprises any one of: 2B4 (Natural killer Cell Receptor 2B4), 4-1BB (Tumor necrosis factor receptor superfamily member 9), CD16 (IgG Fc region Receptor III-A), CD2 (T-cell surface antigen CD2), CD28 (T-cell-specific surface glycoprotein CD28), CD28H (Transmembrane and immunoglobulin domain-containing protein 2), CD3ζ (T-cell surface glycoprotein CD3 zeta chain), CD3 ζ1XX (CD3ζ variant), DAP10 (Hematopoietic cell signal transducer), DAP12 (TYRO protein tyrosine kinase-binding protein), DNAM1 (CD226 antigen), FcERIγ (High affinity immunoglobulin epsilon receptor subunit gamma), IL21R (Interleukin-21 receptor), IL-2Rβ/IL-15RB (Interleukin-2 receptor subunit beta), IL-2Rγ (Cytokine receptor common subunit gamma), IL-7R (Interleukin-7 receptor subunit alpha), KIR2DS2 (Killer cell immunoglobulin-like receptor 2DS2), NKG2D (NKG2-D type II integral membrane protein), NKp30 (Natural cytotoxicity triggering receptor 3), NKp44 (Natural cytotoxicity triggering receptor 2), NKp46 (Natural cytotoxicity triggering receptor 1), CS1 (SLAM family member 7), and CD8 (T-cell surface glycoprotein CD8 alpha chain).

34. The chimeric antigen receptor of claim 32, wherein the endodomain further comprises a second signaling domain, and optionally a third signaling domain; and wherein the first, second and third signaling domains are different.

35. The chimeric antigen receptor of claim 34, wherein the second or the third signaling domain comprises a cytoplasmic domain, or a portion thereof, of 2B4, 4-1BB, CD16, CD2, CD28, CD28H, CD3ζ, DAP10, DAP12, DNAM1, FcERIγ IL21R, (IL-15Rβ), IL-2Rγ, IL-7R, KIR2DS2, NKG2D, NKp30, NKp44, NKp46, CD3 ζ1XX, CS1, or CD8.

36. The chimeric antigen receptor of claim 32, wherein the transmembrane domain comprises an amino acid sequence of a transmembrane region, or a portion thereof, of CD2, CD3D, CD3E, CD3G, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide.

37. The chimeric antigen receptor of claim 32, wherein the ectodomain further comprises:
(i) a signal peptide; and/or
(ii) a spacer/hinge/linker.

38. The chimeric antigen receptor of claim 32, wherein the CAR is comprised in a bi-cistronic construct co-expressing a partial or full length peptide of a cell surface expressed exogenous cytokine or a receptor thereof, wherein the exogenous cytokine or receptor thereof:
(a) comprises at least one of IL2, IL4, IL6, IL7, IL9, IL10, IL11, IL12, IL15, IL18, IL21, and its respective receptor(s); or
(b) comprises at least one of:
(i) co-expression of IL15 and IL15Rα by using a self-cleaving peptide;
(ii) a fusion protein of IL15 and IL15Rα;
(iii) an IL15/IL15Rα fusion protein with intracellular domain of IL15Rα truncated or eliminated;
(iv) a fusion protein of IL15 and membrane bound Sushi domain of IL15Rα;
(v) a fusion protein of IL15 and IL15Rβ;
(vi) a fusion protein of IL15 and common receptor γC, wherein the common receptor γC is native or modified; and
(vii) a homodimer of IL15Rβ,
wherein any one of (b)(i)-(vii) can be co-expressed with a CAR in separate constructs or in a bi-cistronic construct; or
(c) comprises at least one of:
(i) a fusion protein of IL7 and IL7Rα;
(ii) a fusion protein of IL7 and common receptor γC, wherein the common receptor γC is native or modified; and
(iii) a homodimer of IL7Rβ.

39. The chimeric antigen receptor of claim 32, wherein the MR1-CAR is specific for one or more of colorectal cancer, lung cancer, kidney cancer, prostrate cancer, bladder cancer, cervical cancer, melanoma, bone cancer, breast cancer, ovarian cancer or blood cancer.

40. A cell or a population thereof, wherein the cell is an eukaryotic cell, an animal cell, a human cell, an immune cell, an induced pluripotent cell (iPSC), a clonal iPSC or a derivative cell differentiated therefrom, and wherein the cell comprises a polynucleotide encoding at least the chimeric antigen receptor (CAR) of any one of claims 32-39.

41. The cell or a population thereof of claim 40, wherein the cell further comprises:
(i) a polynucleotide encoding a transgenic TCRα chain (tgTCRα); and
(ii) a polynucleotide encoding a transgenic TCRβ chain (tgTCRβ), wherein the tgTCRα chain and the tgTCRβ chain form an exogenous TCR complex (TCR^{exo}) that recognizes a first tumor antigen other than MR1; and optionally,
(iii) one or more additional exogenous polynucleotides comprising a polynucleotide encoding an engager targeting at least a second tumor antigen.

42. The cell or population thereof of claim 41, wherein:
(i) the polynucleotide encoding the tgTCRα chain and the polynucleotide encoding the tgTCRβ chain are comprised in a bi-cistronic construct, and optionally wherein:
(a) the construct is inserted at a constant region of TCRα or TCRβ (TRAC or TRBC);
(b) the insertion of the construct disrupts expression of endogenous TCRα or TCRβ at the insertion site; and/or
(c) expression of the construct is driven by an endogenous promoter of TCR or an exogenous promoter; or
(ii) the polynucleotide encoding the CAR or the engager is inserted at TRAC or TRBC, and optionally wherein:
(a) the insertion of the polynucleotide encoding the CAR or the engager disrupts expression of the endogenous TCRα or TCRβ at the insertion site; and/or
(b) expression of the CAR or the engager is driven by an endogenous promoter of TCR or an exogenous promoter; or
(iii) the polynucleotides encoding the tgTCRα chain and the tgTCRβ chain, the CAR or the engager, or the one or more additional polynucleotides are inserted in one, or more safe harbor loci or selected gene loci.

43. The cell or population thereof of claim 42, (I) wherein the construct and the polynucleotide encoding the CAR or the engager are each inserted at a constant region of TCRα or TCRβ (TRAC or TRBC), but not at the same constant region, thereby disrupting expression of both endogenous TCRα and TCRβ, knocking out the endogenous TCR, and avoiding a mispaired TCR comprising:
(a) the transgenic TCRα and the endogenous TCRβ, or
(b) the transgenic TCRβ and the endogenous TCRα;
or (II) wherein the construct and the polynucleotide encoding the CAR or the engager are each integrated at a locus comprising a safe harbor locus or a selected gene locus.

44. A composition comprising the cell or population thereof of any one of claims 40-43.

45. The composition of claim 44, wherein the cell or population thereof comprises the iPSC derivative effector cell, and wherein the composition further comprises one or more therapeutic agents.

46. Therapeutic use of the composition of claim 44 or 45 by introducing the composition to a subject suitable for adoptive cell therapy, wherein the subject has an autoimmune disorder, a hematological malignancy, a solid tumor, cancer, or a viral infection.

47. The therapeutic use of the composition of claim 46, wherein the subject has colorectal cancer, lung cancer, kidney cancer, prostrate cancer, bladder cancer, cervical cancer, stomach cancer, melanoma, bone cancer, breast cancer, ovarian cancer or blood cancer.

48. A method of enhancing a CAR-T cell function, wherein the CAR-T cell has a first tumor antigen specificity via a CAR, and wherein the method comprises introducing to the CAR-T cell:
(i) a polynucleotide encoding a transgenic TCRα chain (tgTCRα); and
(ii) a polynucleotide encoding a transgenic TCRβ chain (tgTCRβ), wherein the tgTCRα chain and the tgTCRβ chain form an exogenous TCR complex (TCR^{exo}) that has a second tumor antigen specificity;
wherein the CAR-induced tumor killing efficacy of the CAR-T cell is enhanced by expression of TCR^{exo}.

49. The method of claim 48, wherein the CAR-T cell comprises:
(i) an endogenous TCR knockout by disrupting expression of both endogenous TCRα and TCRβ; or
(ii) the CAR is inserted at a constant region of TCRα or TCRβ (TRAC or TRBC), thereby disrupting expression of endogenous TCRα or TCRβ of the CAR-T cell.

50. The method of claim 48 or 49, wherein the method further comprises activating TCR^{exo} using the second tumor antigen recognized by TCR^{exo}, wherein the first tumor antigen specificity and the second tumor antigen specificity are different.

51. The method of claim 48, wherein the step of introducing to the CAR-T cell a polynucleotide further comprises:
differentiating a genetically engineered iPSC to a T cell, wherein the iPSC comprises: (a) the polynucleotide encoding a transgenic TCRα chain (tgTCRα); (b) the polynucleotide encoding a transgenic TCRβ chain (tgTCRβ); and (c) a polynucleotide encoding the CAR having the first tumor antigen specificity, thereby obtaining the CAR-T cell having expression of the TCR^{exo}.

52. The method of claim 51, further comprising:
genomically engineering a clonal iPSC to knock in: (a) the polynucleotide encoding the transgenic TCRα chain (tgTCRα); (b) the polynucleotide encoding the transgenic TCRβ chain (tgTCRβ); and (c) the polynucleotide encoding the CAR having the first tumor antigen specificity, thereby obtaining an engineered iPSC for T cell differentiation.
